(19) United States
(12) Patent Application Publication
(10) Pub. No.: US 2025/0223296 A1
(43) Pub. Date: Jul. 10, 2025

(54) HYDROXYMETHYL AZABICYCLO[2.2.1]HEPTANES AND MEDICAL USES THEREOF

(71) Applicant: ATROGI AB, Stockholm (SE)

(72) Inventors: Ilga Mutule, Jurmala (LV); Tore Bengtsson, Vaxholm (SE); Benjamin Pelcman, Stockholm (SE); Krista Jaunsleine, Liepaja (LV)

(21) Appl. No.: 18/848,949

(22) PCT Filed: Mar. 23, 2023
(86) PCT No.: PCT/EP2023/057531

§ 371 Date: Sep. 20, 2024

(30) Foreign Application Priority Data
Mar. 23, 2022 (GB) 2204085.1

(51) Int. Cl. C07D 487/08; A61K 31/439

(52) U.S. Cl. CPC C07D 487/08; A61K 31/439

(57) ABSTRACT

There are provided herein compounds of formula (I) and pharmaceutically acceptable salts thereof, wherein Y, L, and Z, and the ring containing Q¹ to Q⁵ have meanings as provided in the description. There are also provided herein medical uses of such compounds.

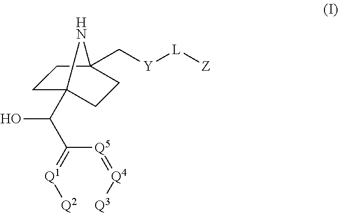

### FIELD OF THE INVENTION

The present invention relates to novel compounds and compositions, and their use in medicine, such as in the treatment of hyperglycemia and disorders characterised by hyperglycemia, such as type 2 diabetes. In particular, the invention relates to novel compounds, compositions and methods for the treatment of conditions such as type 2 diabetes through activation of the β₂-adrenergic receptor. Such compounds are thought to have a beneficial side-effect profile as they do not exert their effect through significant CAMP release.

### BACKGROUND OF THE INVENTION

The listing or discussion of an apparently prior-published document in this specification should not necessarily be taken as an acknowledgement that the document is part of the state of the art or is common general knowledge.

Hyperglycemia, or high blood sugar is a condition in which an excessive amount of glucose circulates in the blood plasma. If not treated, hyperglycemia can be a serious problem, potentially developing into life-threatening conditions such as ketoacidosis. For example, chronic hyperglycemia may cause injury to the heart, and is strongly associated with heart attacks and death in subjects with no coronary heart disease or history of heart failure. There are various causes of hyperglycemia, including diabetes and severe insulin resistance.

Severe insulin resistance (SIR) is a condition wherein the patient experiences very low levels of (or, in extreme cases, no significant) response to insulin. There are several syndromes characterized by SIR, including Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndrome, pseudoacromegaly, and lipodystrophy. The majority of these conditions have genetic causes, such as mutations in the insulin receptor gene. The prevalence for Donohue's syndrome, Rabson-Mendenhall syndrome and Type A syndrome of insulin resistance, has been reported to vary from about 50 reported cases to 1 in 100,000. However, since some diseases are severe and extremely rare, it is likely that many patients do not get diagnosed before they die, particularly in less developed areas of the world. Thus, the exact number of patients with these syndromes is difficult to assess.

The current standard for hyperglycaemia treatment in patients having SIR is a controlled diet, supplemented with drugs affecting insulin receptor sensitivity, such as metformin, or insulin supplement. However, particularly for disorders caused by mutations in the insulin receptor gene, this treatment is not sufficiently effective and ultimately proves unsuccessful.

Diabetes comprises two distinct diseases, type 1 (or insulin-dependent diabetes) and type 2 (insulin-independent diabetes), both of which involve the malfunction of glucose homeostasis. Type 2 diabetes affects more than 400 million people in the world and the number is rising rapidly. Complications of type 2 diabetes include severe cardiovascular problems, kidney failure, peripheral neuropathy, blindness and, in the later stages of the disease, even loss of limbs and, ultimately death. Type 2 diabetes is characterized by insulin resistance in skeletal muscle and adipose tissue, and there is presently no definitive cure. Most treatments used today are focused on remedying dysfunctional insulin signalling or inhibiting glucose output from the liver but many of those treatments have several drawbacks and side effects. There is thus a great interest in identifying novel insulin-independent ways to treat type 2 diabetes.

In type 2 diabetes, the insulin-signalling pathway is blunted in peripheral tissues such as adipose tissue and skeletal muscle. Methods for treating type 2 diabetes typically include lifestyle changes, as well as insulin injections or oral medications to regulate glucose homeostasis. People with type 2 diabetes in the later stages of the disease develop ‘beta-cell failure’ i.e. the inability of the pancreas to release insulin in response to high blood glucose levels. In the later stages of the disease patients often require insulin injections in combination with oral medications to manage their diabetes. Further, most common drugs have side effects including downregulation or desensitization of the insulin pathway and/or the promotion of lipid incorporation in adipose tissue, liver and skeletal muscle. There is thus a great interest in identifying novel ways to treat metabolic diseases including type 2 diabetes that do not include these side effects.

Following a meal, increased blood glucose levels stimulate insulin release from the pancreas. Insulin mediates normalization of the blood glucose levels. Important effects of insulin on glucose metabolism include facilitation of glucose uptake into skeletal muscle and adipocytes, and an increase of glycogen storage in the liver. Skeletal muscle and adipocytes are responsible for insulin-mediated glucose uptake and utilization in the fed state, making them very important sites for glucose metabolism.

The signalling pathway downstream from the insulin receptor has been difficult to understand in detail. In brief, control of glucose uptake by insulin involves activation of the insulin receptor (IR), the insulin receptor substrate (IRS), the phosphoinositide 3-kinase (PI3K) and thus stimulation of phosphatidylinositol (3,4,5)-triphosphate (PIP3), the mammalian target of rapamycin (also called the mechanistic target of rapamycin, mTOR), Akt/PKB (Akt) and TBC1D4 (AS160), leading to translocation of the glucose transporter 4 (GLUT4) to the plasma membrane. Akt activation is considered necessary for GLUT4 translocation.

It should be noted that skeletal muscles constitute a major part of the body weight of mammals and have a vital role in the regulation of systemic glucose metabolism, being responsible for up to 85% of whole-body glucose disposal. Glucose uptake in skeletal muscles is regulated by several intra- and extra-cellular signals. Insulin is the most well studied mediator but others also exist. For example, AMP activated kinase (AMPK) functions as an energy sensor in the cell, which can increase glucose uptake and fatty acid oxidation. Due to the great influence skeletal muscles have on glucose homeostasis it is plausible that additional mechanisms exist. In the light of the increased prevalence of type 2 diabetes, it is of great interest to find and characterize novel insulin-independent mechanisms to increase glucose uptake in muscle cells.

Blood glucose levels may be regulated by both insulin and catecholamines, but they are released in the body in response to different stimuli. Whereas insulin is released in response to the rise in blood sugar levels (e.g. after a meal), epinephrine and norepinephrine are released in response to various internal and external stimuli, such as exercise, emotions and stress, and also for maintaining tissue homeostasis. Insulin is an anabolic hormone that stimulates many processes involved in growth including glucose uptake, glycogen and triglyceride formation, whereas catecholamines are mainly catabolic.

Although insulin and catecholamines normally have opposing effects, it has been shown that they have similar actions on glucose uptake in skeletal muscle (Nevzorova et al., Br. J. Pharmacol, 137, 9, (2002)). In particular, it has been reported that catecholamines stimulate glucose uptake via adrenergic receptors (Nevzorova et al., Br. J. Pharmacol, 147, 446, (2006); Hutchinson, Bengtsson Endocrinology 146, 901, (2005)) to supply muscle cells with an energy-rich substrate. Thus it is likely that in mammals, including humans, the adrenergic and the insulin systems can work independently to regulate the energy needs of skeletal muscle in different situations. Since insulin also stimulates many anabolic processes, including some that promote undesired effects such as stimulation of lipid incorporation into tissues, leading to e.g. obesity, it would be beneficial to be able to stimulate glucose uptake by other means; for example, by stimulation of the adrenergic receptors (ARs).

All ARs are G protein-coupled receptors (GPCRs) located in the cell membrane and characterized by an extracellular N-terminus, followed by seven transmembrane α-helices (TM-1 to TM-7) connected by three intracellular (IL-1 to IL-3) and three extracellular loops (EL-1 to EL-3), and finally an intracellular C-terminus. There are three different classes of ARs, with distinct expression patterns and pharmacological profiles: α₁-, α₂- and β-ARs. The α₁-ARs comprise the α_{1A}, α_{1B} and dip subtypes while α₂-ARs are divided into α_{2A}, α_{2B} and α_{2C}. The β-ARs are also divided into the subtypes β₁, β₂, and β₃, of which β₂-AR is the major isoform in skeletal muscle cells. ARs are G protein coupled receptors (GPCRs) that signal through classical secondary messengers such as cyclic adenosine monophosphate (CAMP) and phospholipase C (PLC).

Many effects occurring downstream of ARs in skeletal muscles have been attributed to classical secondary messenger signalling, such as increase in CAMP levels, PLC activity and calcium levels. Stimulation involving the classical secondary messengers has many effects in different tissues. For example, it increases heart rate, blood flow, airflow in lungs and release of glucose from the liver, which all can be detrimental or be considered unwanted side effects if stimulation of ARs should be considered as a type 2 diabetes treatment. Adverse effects of classical AR agonists are, for example, tachycardia, palpitation, tremor, sweats, agitation and increased glucose levels in the blood (glucose output from the liver). It would thus be beneficial to be able to activate ARs without activating these classical secondary messengers, such as CAMP, to increase glucose uptake in peripheral tissues without stimulating the unwanted side effects.

Glucose uptake is mainly stimulated via facilitative glucose transporters (GLUT) that mediate glucose uptake into most cells. GLUTs are transporter proteins that mediate transport of glucose and/or fructose over the plasma membrane down the concentration gradient. There are fourteen known members of the GLUT family, named GLUT1-14, divided into three classes (Class I, Class II and Class III) dependent on their substrate specificity and tissue expression. GLUT1 and GLUT4 are the most intensively studied isoforms and, together with GLUT2 and GLUT3, belong to Class I which mainly transports glucose (in contrast to Class II that also transports fructose). GLUT1 is ubiquitously expressed and is responsible for basal glucose transport. GLUT4 is only expressed in peripheral tissues such as skeletal muscle, cardiac muscle and adipose tissues. GLUT4 has also been reported to be expressed in, for example, the brain, kidney, and liver. GLUT4 is the major isoform involved in insulin stimulated glucose uptake. The mechanism whereby insulin signalling increases glucose uptake is mainly via GLUT4 translocation from intracellular storage to the plasma membrane. It is known that GLUT4 translocation is induced by stimulation of the β₂-adrenergic receptor.

Thus, a possible treatment of a condition involving dysregulation of glucose homeostasis or glucose uptake in a mammal, such as type 2 diabetes, would involve the activation of the β₂-adrenergic receptor leading to GLUT4 translocation to the plasma membrane and promotion of glucose uptake into skeletal muscle leading to normalization of whole body glucose homeostasis. In addition, it would be advantageous if the treatment does not involve signalling through cAMP as this would lead to a favourable side-effect profile.

### DESCRIPTION OF THE INVENTION

We have now surprisingly found that certain substituted hydroxymethyl azabicyclo[2.2.1]heptanes acting as agonists at the β₂-adrenergic receptor increase glucose uptake in skeletal muscle.

Furthermore, we have found that this effect is not mediated through significant cAMP release, such that many of the commonly described side effects seen with traditional β2-adrenergic agonists (e.g. tachycardia, palpitation, tremor, sweats, agitation, and the like) can be reduced.

The use of such compounds in medicine represents a promising strategy for the treatment of conditions as described herein, e.g. those characterized by high blood sugar levels (i.e. hyperglycemia), such as type 2 diabetes.

### Compounds of the Invention

In a first aspect of the invention, there is provided a compound of formula I

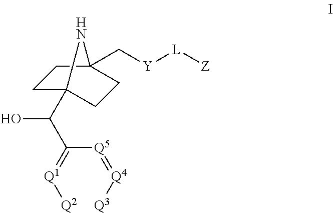

or a pharmaceutically acceptable salt thereof, wherein:
the ring comprising Q¹ to Q⁵ represents
a phenyl optionally substituted with one or more X¹, or
a 5- or 6-membered heteroaryl optionally substituted with one or more X²;
each X¹ and X² independently represents halo, R^{a}, —CN, —N₃, —N(R^{b})R^{c}, —NO₂, —OR^{d}, or —S(O)ₚR^{e};
R^{a} represents C₁-6 alkyl, C₂-6 alkenyl or C₂₋₆ alkynyl optionally substituted by one or more group selected from halo and ═O;
each R^{b} and R^{d} independently represents H or C₁₋₆ alkyl, C₂₋₆ alkenyl or C₂₋₆ alkynyl optionally substituted by one or more group selected from halo and ═O;
each R^{c} and R^{e} independently represents H or C₁₋₆ alkyl, C₂₋₆ alkenyl or C₂₋₆ alkynyl optionally substituted by one or more halo;
or alternatively R^{b} and R^{c} may be linked together to form, together with the nitrogen atom to which they are attached, a 4- to 6-membered ring, which ring optionally contains one further heteroatom and which ring optionally is substituted by one or more group selected from halo and ═O;
Y represents a direct bond or —O—;
L represents
a direct bond,
or a linear or branched C₁₋₁₂ alkylene, linear or branched C₂₋₁₂ alkenylene or linear or branched C₂₋₁₂ alkynylene;
Z represents
(i) C₃₋₈ cycloalkyl, optionally substituted by one or more groups independently selected from G¹,
(ii) aryl optionally substituted by one or more groups independently selected from G², or
(iii) heteroaryl optionally substituted by one or more groups independently selected from G³;
each G¹ independently represents halo, R^{a1}, —CN, —N(R^{b1})R^{c1}, —OR^{d1}, —S(O)ₚR^{e1}, —S(O)_{q}N(R^{f1})R^{g1}, —N(R^{h1})S(O)ₜRⁱ¹ or ═O;
R^{a1} represents C₁₋₆ alkyl, C₂₋₆ alkenyl or C₂₋₆ alkynyl optionally substituted by one or more group selected from halo and ═O;
each R^{b1} and R^{d1} independently represents H or C₁₋₆ alkyl, C₂₋₆ alkenyl or C₂₋₆ alkynyl optionally substituted by one or more group selected from halo and ═O;
each R^{c1}, R^{e1}, R^{f1}, R^{g1} and R^{h1} independently represents H or C₁₋₆ alkyl, C₂₋₆ alkenyl or C₂₋₆ alkynyl optionally substituted by one or more halo;
Rⁱ¹ represents C₁₋₆ alkyl, C₂₋₆ alkenyl or C₂₋₆ alkynyl optionally substituted by one or more halo;
or alternatively any of R^{b1} and R^{c1} and/or R^{f1} and R^{g1} may be linked together to form, together with the nitrogen atom to which they are attached, a 4- to 6-membered ring, which ring optionally contains one further heteroatom and which ring optionally is substituted by one or more groups independently selected from halo and C₁₋₃ alkyl optionally substituted by one or more group selected from halo and ═O;
each G² and G³ independently represents halo, R^{a2}, —CN, —N₃, —N(R^{b2})R^{c2}, —NO₂, —OR^{d2}, —S(O)ₚR^{e2}, —S(O)_{q}N(R^{f2}R^{g2}) or —N(R^{h2})S(O)ₜRⁱ²;
R^{a2} represents C₁₋₆ alkyl, C₂₋₆ alkenyl or C₂₋₆ alkynyl optionally substituted by one or more group selected from halo and ═O;
each R^{b2} and R^{d2} independently represents H or C₁₋₆ alkyl, C₂₋₆ alkenyl or C₂₋₆ alkynyl optionally substituted by one or more group selected from halo and ═O;
each R^{c2}, R^{e2}, R^{f2}, and R^{g2} independently represents H or C₁₋₆ alkyl, C₂₋₆ alkenyl or C₂₋₆ alkynyl optionally substituted by one or more halo;
R^{h2} independently represents H or C₁₋₆ alkyl, C₂₋₆ alkenyl or C₂₋₆ alkynyl optionally substituted by aryl and/or one or more halo, wherein the aryl is optionally substituted by one or more groups independently selected from G⁴;
Rⁱ² represents C₁₋₆ alkyl, C₂₋₆ alkenyl or C₂₋₆ alkynyl optionally substituted by one or more halo;
or alternatively any of R^{b2} and R^{c2} and/or R^{f2} and R^{g2} may be linked together to form, together with the nitrogen atom to which they are attached, a 4- to 6-membered ring, which ring optionally contains one further heteroatom and which ring optionally is substituted by one or more groups independently selected from halo and C₁₋₃ alkyl optionally substituted by one or more group selected from halo and ═O;
each G⁴ independently represents halo, R^{a3}, —CN, —N₃, —N(R^{b3})R^{c3}, —NO₂, —OR^{d3}, —S(O), R^{e3}, —S(O)_{q}N(R^{f3}R^{g3}) or —N(R^{h3})S(O)+Rⁱ³;
R^{a3} represents C₁₋₆ alkyl, C₂₋₆ alkenyl or C₂₋₆ alkynyl optionally substituted by one or more group selected from halo and ═O;
each R^{b3} and R^{d3} independently represents H or C₁₋₆ alkyl, C₂₋₆ alkenyl or C₂₋₆ alkynyl optionally substituted by one or more groups selected from halo and ═O;
each R^{c3}, R^{e3}, R^{f3}, R^{g3} and R^{h3} independently represents H or C₁₋₆ alkyl, C₂₋₆ alkenyl or C₂₋₆ alkynyl optionally substituted by one or more halo;
Rⁱ³ represents C₁₋₆ alkyl, C₂₋₆ alkenyl or C₂₋₆ alkynyl optionally substituted by one or more halo;
each p independently represents 0, 1 or 2;
each q independently represents 1 or 2;
each t independently represents 1 or 2;
wherein, unless otherwise stated, alkyl, alkenyl and alkynyl groups may be linear or branched, and alkyl and alkenyl groups may also be cyclic or part-cyclic, as appropriate, which compounds (including pharmaceutically acceptable salts) may be referred to herein as “compounds of the invention”.

For the avoidance of doubt, the skilled person will understand that references herein to compounds of particular aspects of the invention (such as the first aspect of the invention, e.g. compounds of formula I) will include references to all embodiments and particular features thereof, which embodiments and particular features may be taken in combination to form further embodiments.

Unless indicated otherwise, all technical and scientific terms used herein have the same meaning as commonly understood by one of ordinary skill in the art to which this invention pertains.

Pharmaceutically acceptable salts include acid addition salts and base addition salts, each of which may be in the form of salts in varying ratios of compound to counter ion (e.g. including hemi salts). Such salts may be formed by conventional means, for example by reaction of a free acid or a free base form of a compound comprised in the formulations of the invention with one or more equivalents of an appropriate acid or base, optionally in a solvent, or in a medium in which the salt is insoluble, followed by removal of said solvent, or said medium, using standard techniques (e.g. by rotary evaporation under reduced pressure, by freeze-drying or by filtration). Salts may also be prepared by exchanging a counter-ion of a compound comprised in the formulations of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, hemi-tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, 1-naphtoate, 2-naphtoate, 1-hydroxy-2-naphtoate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1,2-ethane-disulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

Particular pharmaceutically acceptable salts that may be mentioned include hydrochloride (HCl) salts. Other pharmaceutically acceptable salts that may be mentioned include acetate salts.

For the avoidance of doubt, compounds of the first aspect of the invention may exist as solids, and thus the scope of the invention includes all amorphous, crystalline and part crystalline forms thereof, and may also exist as oils. Where compounds of the first aspect of the invention exist in crystalline and part crystalline forms, such forms may include solvates, which are included in the scope of the invention. Compounds of the first aspect of the invention may also exist in solution.

Compounds of the first aspect of the invention may contain double bonds and may thus exist as E (entgegen) and Z (zusammen) geometric isomers about each individual double bond. All such isomers and mixtures thereof are included within the scope of the invention.

Compounds of the first aspect of the invention may also exhibit tautomerism. All tautomeric forms and mixtures thereof are included within the scope of the invention.

Compounds of the first aspect of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired optical isomers may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution); for example, with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-z} alkyl or alkylene groups (where z is the upper limit of the range) defined herein may be straight-chain or, when there is a sufficient number (i.e. a minimum of three) of carbon atoms, be branched-chain. When there is sufficient number of carbon atoms (e.g. for alkyl groups, a minimum of three), such groups may also be cyclic or part-cyclic.

Unless otherwise specified, C_{2-z} alkenyl or alkenylene groups (where z is the upper limit of the range) defined herein may be straight-chain or, when there is a sufficient number (i.e. a minimum of three) of carbon atoms, be branched-chain. When there is sufficient number of carbon atoms (e.g. for alkenyl groups, a minimum of five), such groups may also be cyclic or part-cyclic.

Unless otherwise specified, C_{2-z} alkynyl or alkynylene groups (where z is the upper limit of the range) defined herein may be straight-chain or, when there is a sufficient number (i.e. a minimum of four) of carbon atoms, be branched-chain.

For the avoidance of doubt, the skilled person will understand that the term alkyl will refer to saturated hydrocarbon moieties, whereas the term alkenyl will refer to unsaturated hydrocarbon moieties containing at least one carbon-carbon double bond and the term alkynyl will refer to unsaturated hydrocarbon moieties containing at least one carbon-carbon triple bond.

For the avoidance of doubt, the skilled person will understand that references to alkylene, alkenylene and alkynylene groups will refer to such alkyl, alkenyl and alkynyl groups, respectively, wherein such groups are present as a linker between two other groups.

Particular alkyl, alkenyl and alkynyl groups that may be mentioned include linear or branched groups. More particular alkyl, alkenyl and alkynyl groups that may be mentioned include linear groups.

Particular alkylene, alkenylene and alkynylene groups that may be mentioned include linear groups.

As described herein, the ring comprising Q¹ to Q⁵ (which may be referred to as ring Q) represents a phenyl optionally substituted with one or more X¹, or a 5- or 6-membered heteroaryl optionally substituted with one or more X².

Therefore, it may be stated that each of Q¹ to Q⁵ independently represent carbon, a heteroatom or a direct bond such that the ring comprising Q¹ to Q⁵ represents: a phenyl optionally substituted with one or more X¹, or a 5- or 6-membered heteroaryl optionally substituted with one or more X².

As such, the skilled person will understand that representing Q¹ to Q⁵ the ring may comprise, in addition to carbon atoms, one or more heteroatom, so as to form suitable heteroaryl groups as known to those skilled in the art. Moreover, the skilled person will understand that where the ring containing Q¹ to Q⁵ is 5-membered, one of Q¹ to Q⁵ (e.g. Q⁵) will represent a direct bond (i.e. that group will not be present).

Thus, the skilled person will understand that Q¹ to Q⁵ will either:
each represent carbon atoms, so as to form a phenyl group; or
together represent carbon atoms, one or more heteroatom and, where the ring containing Q¹ to Q⁵ is 5-membered, a direct bond, so as to form suitable heteroaryl groups as known to those skilled in the art.

For the avoidance of doubt, the depiction of the ring containing the Q¹ to Q⁵ groups as comprising conjugated double bonds (such as in formula I) will be understood to indicate that the ring is aromatic, which may also be indicated by replacing the double bonds with a circle within the ring.

Various heteroaryl groups will be well-known to those skilled in the art, such as pyridinyl, pyridonyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl and the like. The oxides of heteroaryl/heteroaromatic groups are also embraced within the scope of the invention (e.g. the N-oxide).

In particular, the term heteroaryl (or heteroaromatic) includes references to 5-membered or 6-membered heteroaromatic groups containing at least one N atom and optionally one additional heteroatom selected (e.g. from oxygen, nitrogen and/or sulphur). Particular heteroaryl groups that may be mentioned include those comprising, in the heteroaryl ring, at least one N atom.

Particular heteroaryl groups (e.g. representing ring Q) that may be mentioned include thiazolyl (e.g. thiazol-4-yl and thiazol-5-yl, also thiazol-2-yl), pyrimidinyl (e.g. pyrimidin-4-yl or pyrimidin-5-yl) and pyridonyl (e.g. pyridon-4-yl or pyridon-5-yl). For the avoidance of doubt, the skilled person will understand that pyridonyl groups may exist as the aromatic tautomers thereof, i.e. as hydroxy pyridinyl groups.

More particular heteroaryl groups (e.g. representing ring Q) that may be mentioned include pyridinyl (e.g. pyridin-2-yl, pyridin-3-yl and pyridine-4-yl, such as pyridin-3-yl).

As will be understood by those skilled in the art, substituents on heteroaryl groups may, as appropriate, be located on any atom in the ring system, including a heteroatom (i.e. a N atom, where the valency of that atom allows). In such circumstances, the skilled person will understand that reference to the substituent being present “as appropriate” will indicate that certain substituents may only be present in positions wherein the presence of such a substituent is chemically allowable, as understood by those skilled in the art.

For example, where X² is present on a secondary N atom (i.e. —N—; in which case, the X² group may instead be referred to as X²a), each X² may independently represent R^{a}, CN or —S(O)ₚR^{e}. Similarly, where X² is present on a C atom (in which case, it may be referred to as X²b), each X² may independently represent halo, R^{a}, —CN, —N₃, —N(R^{b})R^{c}, —NO₂, —ONO₂, —OR^{d} or —S(O)ₚR^{e}.

For the avoidance of doubt, the skilled person will understand that the identities of Q¹ to Q⁵ will be selected such that the resulting heteroaryl is a suitable heteroaryl as known to those skilled in the art; for example, with the definitions of Q¹ to Q⁵ corresponding to C (i.e. present as CH or CX², as appropriate), N (as a tertiary N or a secondary N present as NH or NX², as appropriate), O or a direct bond, so as to form 5- or 6-membered heteroaryl groups as known in the art (such as those described herein).

For example, in relation to ring Q, particular heteroaryl groups that may be mentioned include those in which one or two of Q¹ to Q⁵ represent N (particularly where, if two represent N, those groups are non-adjacent) and the others of Q¹ to Q⁵ represent C (i.e. as CH or CX², as appropriate).

For example, the skilled person will understand that in 5-membered heteroaryl rings there can only be one O or S, but up to four N atoms (with up to four heteroatoms in total), which may be present as N or NX¹ (particularly, with up to one being present as NH or NX² and the remainder being present as N), as appropriate. Similarly, in a 6-membered heteroaryl ring there will be no O or S present in the ring but up to four (e.g. one or two, such as one) N atoms, which will be present as N.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulphur (e.g. oxygen, nitrogen and sulphur, such as nitrogen).

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more X¹ groups are present, those X¹ groups may be the same or different. Similarly, where two or more X¹ groups are present and each represent halo, the halo groups in question may be the same or different. Likewise, when more than one R^{a} is present and each independently represents C₁₋₆ alkyl substituted by one or more halo, the identities of each halo are in no way interdependent.

The skilled person will appreciate that compounds of the invention that are the subject of this invention include those that are stable. That is, compounds of the invention include those that are sufficiently robust to survive isolation, e.g. from a reaction mixture, to a useful degree of purity.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

In certain embodiments that may be mentioned:
the ring comprising Q¹ to Q⁵ represents
a phenyl optionally substituted with one or more X¹, or
a 5- or 6-membered heteroaryl optionally substituted with one or more X²;
each X¹ and X² independently represents halo, R^{a}, —CN, —N₃, —N(R^{b})R^{c}, —NO₂, —OR^{d}, or —S(O)ₚR^{e};
R^{a} represents C₁₋₆ alkyl, C₂₋₆ alkenyl or C₂₋₆ alkynyl optionally substituted by one or more group selected from halo and ═O;
each R^{b} and R^{d} independently represents H or C₁₋₆ alkyl, C₂₋₆ alkenyl or C₂₋₆ alkynyl optionally substituted by one or more group selected from halo and ═O;
each R^{c} and R^{e} independently represents H or C₁₋₆ alkyl, C₂₋₆ alkenyl or C₂₋₆ alkynyl optionally substituted by one or more halo;
or alternatively R^{b} and R^{c} may be linked together to form, together with the nitrogen atom to which they are attached, a 4- to 6-membered ring, which ring optionally contains one further heteroatom and which ring optionally is substituted by one or more group selected from halo and ═O;
Y represents a direct bond or —O—;
L represents
a direct bond,
or a linear or branched C₁₋₁₂ alkylene, linear or branched C₂₋₁₂ alkenylene or linear or branched C₂₋₁₂ alkynylene;
Z represents
(i) C₃₋₈ cycloalkyl, optionally substituted by one or more groups independently selected from G¹,
(ii) aryl optionally substituted by one or more groups independently selected from G², or
(iii) heteroaryl optionally substituted by one or more groups independently selected from G³;
each G¹ independently represents halo, R^{a1}, —CN, —N(R^{b1})R^{c1}, —OR^{d1}, —S(O)ₚR^{e1}, —S(O)_{q}N(R^{f1})R^{g1}, —N(R^{h1})S(O)ₜRⁱ¹ or ═O;
R^{a1} represents C₁₋₆ alkyl, C₂₋₆ alkenyl or C₂₋₆ alkynyl optionally substituted by one or more group selected from halo and ═O;
each R^{b1} and R^{d1} independently represents H or C₁₋₆ alkyl, C₂₋₆ alkenyl or C₂₋₆ alkynyl optionally substituted by one or more group selected from halo and ═O;
each R^{c1}, R^{e1}, R^{f1}, R^{g1} and R^{h1} independently represents H or C₁₋₆ alkyl, C₂₋₆ alkenyl or C₂₋₆ alkynyl optionally substituted by one or more halo;
Rⁱ¹ represents C₁₋₆ alkyl, C₂₋₆ alkenyl or C₂₋₆ alkynyl optionally substituted by one or more halo;
or alternatively any of R^{b1} and Rel and/or R^{f1} and R^{g1} may be linked together to form, together with the nitrogen atom to which they are attached, a 4- to 6-membered ring, which ring optionally contains one further heteroatom and which ring optionally is substituted by one or more groups independently selected from halo and C₁₋₃ alkyl optionally substituted by one or more group selected from halo and ═O;
each G² and G³ independently represents halo, R^{a2}, —CN, —N₃, —N(R^{b2})R^{c2}, —NO₂, —OR^{d2}, —S(O)ₚR^{e2}, —S(O)_{q}N(R^{f2}R^{g2}) or —N(R^{h2})S(O)ₜRⁱ²;
R^{a2} represents C₁₋₆ alkyl, C₂₋₆ alkenyl or C₂₋₆ alkynyl optionally substituted by one or more group selected from halo and ═O;
each R^{b2} and R^{d2} independently represents H or C₁₋₆ alkyl, C₂₋₆ alkenyl or C₂₋₆ alkynyl optionally substituted by one or more group selected from halo and ═O;
each R^{c2}, R^{e2}, R^{f2}, R^{g2} and R^{h2} independently represents H or C₁₋₆ alkyl, C₂₋₆ alkenyl or C₂₋₆ alkynyl optionally substituted by one or more halo;
Rⁱ² represents C₁₋₆ alkyl, C₂₋₆ alkenyl or C₂₋₆ alkynyl optionally substituted by one or more halo;
or alternatively any of R^{b2} and R^{c2} and/or R^{f2} and R^{g2} may be linked together to form, together with the nitrogen atom to which they are attached, a 4- to 6-membered ring, which ring optionally contains one further heteroatom and which ring optionally is substituted by one or more groups independently selected from halo and C₁₋₃ alkyl optionally substituted by one or more group selected from halo and ═O;
each p independently represents 0, 1 or 2;
each q independently represents 1 or 2;
each t independently represents 1 or 2.

In a particular embodiment (i.e. a particular embodiment of the first aspect of the invention), when Y is —O— and L is a direct bond, Z represents
C₃₋₈ cycloalkyl, optionally substituted by one or more groups independently selected from G¹, or
heteroaryl optionally substituted by one or more groups independently selected from G³.

In a more particular embodiment, when Y is —O— and L is a direct bond, Z represents C₃₋₈ cycloalkyl, optionally substituted by one or more groups independently selected from G¹.

In certain embodiments, when Y is —O— and L is a direct bond, Z does not represent optionally substituted aryl.

In certain embodiments, when Y is —O— and L is a direct bond, Z does not represent substituted aryl.

In a particular embodiment, the ring comprising Q¹ to Q⁵ is optionally substituted with one to three (e.g. one or two, such as one) X¹ or X² groups, as appropriate.

In a particular embodiment, the ring comprising Q¹ to Q⁵ represents
a phenyl optionally substituted with one or more (e.g. one or two, such as one) X¹, or
a 6-membered heteroaryl optionally substituted with one or more (e.g. one or two, such as one) X².

In certain embodiments, the ring comprising Q¹ to Q⁵ represents a 6-membered heteroaryl optionally substituted with one or more (e.g. one or two, such as one) X².

In a more particular embodiment, the ring comprising Q¹ to Q⁵ represents
a phenyl optionally substituted with one or more (e.g. one or two, such as one) X¹, or
a pyridyl optionally substituted with one or more (e.g. one or two, such as one) X².

In certain embodiments, the ring comprising Q¹ to Q⁵ represents a pyridyl optionally substituted with one or more (e.g. one or two, such as one) X².

In a yet more particular embodiment, the ring comprising Q¹ to Q⁵ represents a phenyl optionally substituted with one or more (e.g. one or two, such as one) X¹, or a pyridin-3-yl optionally substituted with one or more (e.g. one or two, such as one) X².

In certain embodiments, the ring comprising Q¹ to Q⁵ represents a pyridin-3-yl optionally substituted with one or more (e.g. one or two, such as one) X².

Thus, in particular embodiments, the ring comprising Q¹ to Q⁵ may instead be represented as one of the following groups:

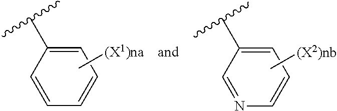

wherein X¹ and X² are as defined herein, na represents 0 to 5 (e.g. 0 to 2, such as 0 or 1) and nb represents 0 to 4 (e.g. 0 to 2, such as 0 or 1).

For the avoidance of doubt, where a bond depicted in a fragment of a molecule as described herein is terminated with a wavy line, that bond will be understood to be the point of attachment to the remainder of the molecule.

In a particular embodiment, each X¹ and X² independently represents halo (e.g. C₁ or F), R^{a}, —CN, —N(R^{b})R^{c} or —OR^{d}.

In particular embodiments:
each R^{a} independently represents C₁₋₆ alkyl (such as C₁₋₃ alkyl, e.g. methyl) optionally substituted by one or more group selected from halo and ═O; and/or (e.g. and) each R^{b}, R^{e} and R^{d} independently represent H or C₁₋₆ alkyl (such as C₁₋₃ alkyl, e.g. methyl) optionally substituted by one or more group selected from halo and ═O.

In a particular embodiment, each X¹ and X² independently represents halo (e.g. F or Cl, such as F), —CH₃, —CF₃, —CN, —NH₂ or —OH.

In a more particular embodiment, each X¹ and X² independently represents halo (e.g. F or Cl, such as F), —CH₃, —CF₃ or —CN.

In a more particular embodiment, each X¹ and X² independently represents F.

In particular embodiments, where the ring comprising Q¹ to Q⁵ represents a phenyl optionally substituted with one or more (e.g. one or two, such as one) X¹, or a 6-membered heteroaryl optionally substituted with one or more (e.g. one or two, such as one) X², such as wherein at least one X¹ or X² group, as appropriate and where present, is present in the 3-position relative to the point of attachment to the essential C (OH) group (which may be referred to as the 5-position of a pyridinyl, such as a pyridin-3-yl).

In a more particular embodiment, where the ring comprising Q¹ to Q⁵ represents a phenyl optionally substituted with one or more (e.g. one or two, such as one) X¹, or a pyridinyl optionally substituted with one or more (e.g. one or two, such as one) X², at least one X¹ or X² group, as appropriate and where present, is present in the 3-position of the phenyl and the 5-position of a pyridin-3-yl.

In a yet more particular embodiment, where the ring comprising Q¹ to Q⁵ represents a phenyl optionally substituted with one or more (e.g. one or two, such as one) X¹, or a pyridin-3-yl optionally substituted with one or more (e.g. one or two, such as one) X², at least one X¹ or X² group, as appropriate and where present, is present in the 3-position of the phenyl and the 5-position of the pyridin-3-yl.

In an alternative embodiment, where the ring comprising Q¹ to Q⁵ represents a phenyl optionally substituted with one or more (e.g. one or two, such as one) X¹, or a pyridin-3-yl optionally substituted with one or more (e.g. one or two, such as one) X², at least one X¹ or X² group, as appropriate and where present, is present in the 2- or 3-position of the phenyl and the 5-position of the pyridin-3-yl.

In a yet more particular embodiment, the ring comprising Q¹ to Q⁵ represents a phenyl substituted with one or more (e.g. one or two, such as one) X¹, or a pyridin-3-yl substituted with one or more (e.g. one or two, such as one) X², wherein at least one X¹ or X² group, as appropriate, is present in the 3-position of the phenyl and the 5-position of the pyridin-3-yl.

In an alternative embodiment, the ring comprising Q¹ to Q⁵ represents a phenyl substituted with one or more (e.g. one or two, such as one) X¹, or a pyridin-3-yl substituted with one or more (e.g. one or two, such as one) X², wherein at least one X¹ or X² group, as appropriate, is present in the 2- or 3-position of the phenyl and the 5-position of the pyridin-3-yl.

In a yet more particular embodiment, the ring comprising Q¹ to Q⁵ represents
a phenyl substituted with one X¹, or
a pyridin-3-yl substituted with one X²,
wherein the X¹ or X² group, as appropriate, is present in the 3-position of the phenyl and the 5-position of the pyridin-3-yl.

In an alternative embodiment, the ring comprising Q¹ to Q⁵ represents
a phenyl substituted with one X¹, or
a pyridin-3-yl substituted with one X²,
wherein the X¹ or X² group, as appropriate, is present in the 2- or 3-position of the phenyl and the 5-position of the pyridin-3-yl.

Thus, in particular embodiments, the ring comprising Q¹ to Q⁵ may instead be represented as one of the following groups:

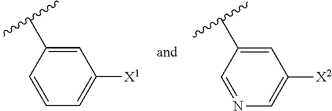

wherein X¹ and X² are as defined herein.

In alternative embodiments, the ring comprising Q¹ to Q⁵ may instead be represented as one of the following groups:

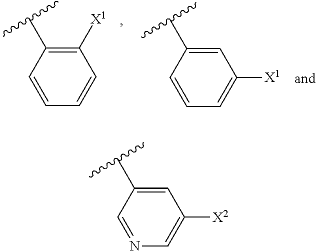

wherein X¹ and X² are as defined herein.

As described herein, particular X¹ and X² groups that may be mentioned include F.

Thus, in particular embodiments, the ring comprising Q¹ to Q⁵ may instead be represented as one of the following groups:

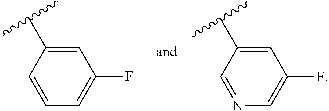 [SMILES: *c1cccc(F)c1 ; *c1cncc(F)c1]

In alternative embodiments, the ring comprising Q¹ to Q⁵ may instead be represented as one of the following groups:

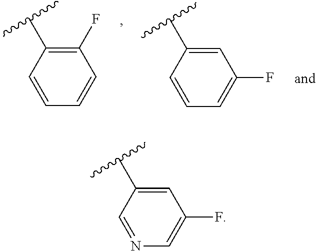 [SMILES: *c1ccccc1F ; *c1cccc(F)c1 ; *c1cncc(F)c1]

In particular embodiments, L represents
a direct bond,
or a linear or branched C₁₋₁₂ alkylene.

In more particular embodiments, L represents
a direct bond,
or a linear or branched C₁₋₆ alkylene.

In more particular embodiments, L represents
a direct bond,
or a linear or branched C₁₋₃ alkylene.

In more particular embodiments, L represents
a direct bond,
or a linear or branched C₁₋₂ alkylene.

In yet more particular embodiments, L represents
a direct bond,
or a methylene.

In particular embodiments, the group-Y-L- as depicted (and in the orientation shown) represents a direct bond (i.e. both Y and L represent a direct bond), —O—CH₂— or —CH₂—.

For the avoidance of doubt, in certain embodiments, Y represents a direct bond.

In certain embodiments, L represents
a direct bond,
or a linear or branched C₁₋₁₂ alkylene, or a linear or branched C₂₋₁₂ alkenylene.

In certain embodiments, L represents
a direct bond,
or a linear or branched C₁₋₃ alkylene, or a linear or branched C₂₋₃ alkenylene.

In certain embodiments, L represents
a direct bond,
or a linear C₁₋₂ alkylene, or a linear C₂ alkenylene.

In certain embodiments:
Y represents a direct bond; and
L represents a linear C₁₋₂ alkylene or a linear C₂ alkenylene.

In certain embodiments:
Y represents a direct bond; and
L represents a linear C₂ alkenylene.

In particular embodiments, Z represents
(i) C₃₋₈ cycloalkyl, optionally substituted by one or more (e.g. one or two, such as one) groups independently selected from G¹, or
(ii) aryl (e.g. phenyl) optionally substituted by one or more (e.g. one or two, such as one) groups independently selected from G².

In alternative embodiments, Z represents
(i) C₃₋₈ cycloalkyl, optionally substituted by one or more (e.g. one or two, such as one) groups independently selected from G¹,
(ii) aryl (e.g. phenyl) optionally substituted by one or more (e.g. one or two, such as one) groups independently selected from G²; or
(iii) a 6-membered heteroaryl optionally substituted by one or more groups independently selected from G³.

In certain embodiments, Z represents C₃₋₈ cycloalkyl, optionally substituted by one or more (e.g. one or two, such as one) groups independently selected from G¹.

In more particular embodiments, Z represents
(i) C₅₋₆ cycloalkyl, optionally substituted by one or more (e.g. one or two, such as one) groups independently selected from G¹, or
(ii) aryl (e.g. phenyl) optionally substituted by one or more (e.g. one or two, such as one) groups independently selected from G².

In alternative embodiments, Z represents
(i) C₅₋₆ cycloalkyl, optionally substituted by one or more (e.g. one or two, such as one) groups independently selected from G¹,
(ii) aryl (e.g. phenyl) optionally substituted by one or more (e.g. one or two, such as one) groups independently selected from G², or
(iii) pyridyl optionally substituted by one or more groups independently selected from G³.

In certain embodiments, Z represents C₅₋₆ cycloalkyl, optionally substituted by one or more (e.g. one or two, such as one) groups independently selected from G¹.

In more particular embodiments, Z represents
(i) C₅₋₆ cycloalkyl, optionally substituted by one or more (e.g. one or two, such as one) groups independently selected from G¹, or
(ii) phenyl optionally substituted by one or more (e.g. one or two, such as one) groups independently selected from G².

In alternative embodiments, Z represents
(i) C₅₋₆ cycloalkyl, optionally substituted by one or more (e.g. one or two, such as one) groups independently selected from G¹,
(ii) phenyl optionally substituted by one or more (e.g. one or two, such as one) groups independently selected from G², or
(iii) pyridin-3-yl optionally substituted by one or more groups independently selected from G³.

In certain embodiments, Z represents C₅₋₆ cycloalkyl, optionally substituted by one or more (e.g. one or two, such as one) groups independently selected from G¹.

In yet more particular embodiments, Z represents
(i) cyclohexyl, optionally substituted by one or more (e.g. one or two, such as one) groups independently selected from G¹, or
(ii) phenyl optionally substituted by one or more (e.g. one or two, such as one) groups independently selected from G².

In certain embodiments, Z represents cyclohexyl, optionally substituted by one or more (e.g. one or two, such as one) groups independently selected from G¹.

Thus, in particular embodiments, Z may instead be represented as one of the following groups:

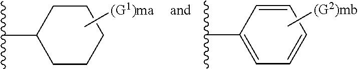

wherein G¹ and G² are as defined herein, ma represents 0 to 11 (e.g. 0 to 3, such as 0 or 1) and mb represents 0 to 5 (e.g. 0 to 3, such as 0 or 1).

In further such embodiments, Z may also (or instead) be represented as:

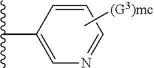

wherein G³ is as defined herein, mc represents 0 to 4 (e.g. 0 to 3, such as 0 or 1, e.g. 1).

In particular embodiments, at least one (such as one) G¹ or G² group, as appropriate, is present.

In particular embodiments, at least one (such as one) G¹, G² or G³ group, as appropriate, is present.

For the avoidance of doubt, in particular embodiments:
each G² and G³ independently represents halo, R^{a2}, —CN, —N₃, —N(R^{b2})R^{c2}, —NO₂, —OR^{d2}, —S(O)ₚR^{e2}, —S(O)_{q}N(R^{f2}R^{g2}) or —N(R^{h2})S(O)ₜRⁱ²;
R^{a2} represents C₁₋₆ alkyl, C₂₋₆ alkenyl or C₂₋₆ alkynyl optionally substituted by one or more group selected from halo and ═O;
each R^{b2} and R^{d2} independently represents H or C₁₋₆ alkyl, C₂₋₆ alkenyl or C₂₋₆ alkynyl optionally substituted by one or more group selected from halo and ═O; and
each R^{c2}, R^{e2}, R^{f2}, R^{g2} and R^{h2} independently represents H or C₁₋₆ alkyl, C₂₋₆ alkenyl or C₂₋₆ alkynyl optionally substituted by one or more halo.

In particular embodiments, ma and mb each represent 1.

In particular embodiments:
each G¹ independently represents halo (e.g. F), R^{a1}, —N(R^{b1})R^{c1}, —OR^{d1}, —S(O)ₚR^{e1}, —S(O)_{q}N(R^{f1})R^{g1} or —N(R^{h1})S(O)ₜRⁱ¹; and/or
each G² and/or G³ independently represents halo (e.g. F or Cl), R^{a2}, —CN, —N(R^{b2})R^{e2}, —OR^{d2}, —S(O)ₚR^{e2}, —S(O)_{q}N(R^{f2})R^{g2} or —N(R^{h2})S(O)ₜRⁱ².

In more particular embodiments:
each G¹ independently represents halo (e.g. F), —N(R^{b1})R^{c1}, —OR^{d1} or —N(R^{h1})S(O)ₜRⁱ¹; and/or each G² and/or G³ independently represents halo (e.g. F or Cl), R^{a2}, —N(R^{b2})R^{c2}, —OR^{d2} or —N(R^{h2})S(O)ₜRⁱ².

In more particular embodiments:
each G¹ independently represents —N(R^{b1})R^{c1}, —OR^{d1} or —N(R^{h1})S(O)ₜRⁱ¹; and/or
each G² and/or G³ independently represents halo (e.g. F or Cl), R^{a2}, —N(R^{b2})R^{c2}, —OR^{d2} or —N(R^{h2})S(O)ₜRⁱ².

In more particular embodiments:
each G¹ independently represents —N(R^{b1})R^{c1} or —OR^{d1}; and/or
each G² and/or G³ independently represents halo (e.g. F or Cl, such as F), R^{a2}, —N(R^{b2})R^{c2} or —OR^{d2}.

In particular embodiments:
each R^{a1} and R^{a2} represents C₁₋₃ alkyl optionally substituted by one or more halo (such as —CH₃, —CHF₂ or —CF₃);
each R^{b1} and R^{b2} represents H or C₁₋₃ alkyl optionally substituted by one or more group selected from halo and ═O (such as —CH₃, —C(O)CH₃ or —C(O)CF₃);
each R^{d1} and R^{d2} represents H or C₁₋₃ alkyl optionally substituted by one or more group selected from halo and ═O (such as —CH₃, —CHF₂, —CF₃, —C(O)CH₃ or —C(O)CF₃);
each R^{c1}, R^{f1}, R^{g1}, R^{h1}, R^{c2}, R^{f2}, R^{g2} and R^{h2} independently represents H or C₁₋₃ alkyl (such as —CH₃); and/or
each R^{e1}, Rⁱ¹, R^{e2} and Rⁱ² independently represents C₁₋₃ alkyl (e.g. C₁ alkyl) optionally substituted by one or more halo (such as —CH₃, —CHF₂ or —CF₃).

For example, in particular embodiments:
each G¹ independently represents —N(H)R^{b1}, —OR^{d1} or —N(H)S(O)ₜRⁱ²; and/or
each G² and/or G³ independently represents halo (e.g. F or Cl), N (H)R^{b2}, —OR^{d2} or —N(H)S(O)ₜRⁱ²,
such as wherein each R^{b1} and R^{b2} represents H or C₁₋₃ alkyl (such as —CH₃), each R^{d1} and R^{d2} represents H or C₁₋₃ alkyl optionally substituted by one or more halo (such as —CH₃ or —CF₃), and each Rⁱ¹ and Rⁱ² represents C₁₋₃ alkyl optionally substituted by one or more halo (such as —CH₃).

In further embodiments:
each R^{a1} and R^{a2} represents C₁₋₃ alkyl optionally substituted by one or more halo (such as —CH₃, —CHF₂ or —CF₃);
each R^{b1} and R^{b2} represents H or C₁₋₃ alkyl optionally substituted by one or more group selected from halo and ═O (such as —CH₃, —C(O)CH₃ or —C(O)CF₃);
each R^{d1} and R^{d2} represents H or C₁₋₃ alkyl optionally substituted by one or more group selected from halo and ═O (such as —CH₃, —CHF₂, —CF₃, —C(O)CH₃ or —C(O)CF₃);
each R^{c1}, R^{f1}, R^{g1}, R^{h1}, R^{c2}, R^{f2} and R^{g2} independently represents H or C₁₋₃ alkyl (such as —CH₃);
each R^{h2} independently represents H or C₁₋₃ alkyl (such as —CH₃) optionally substituted by aryl, wherein the aryl is optionally substituted by one or more groups independently selected from G⁴; and/or
each R^{e1}, Rⁱ¹, R^{e2} and Rⁱ² independently represents C₁₋₃ alkyl (e.g. C₁ alkyl) optionally substituted by one or more halo (such as —CH₃, —CHF₂ or —CF₃).

In more particular embodiments:
each G¹ independently represents —N(H)C(O)CH₃, —N(H)C(O)CF₃, —OCH₃, —N(H)S(O)₂CH₃ or —N(H)S(O)₂CF₃; and/or
each G² and/or G³ (e.g. G²) independently represents F, Cl, —N(H)C(O)CH₃, —N(H)C(O)CF₃, —OCH₃, —N(H)S(O)₂CH₃ or —N(H)S(O)₂CF₃.

In further embodiments:
each G¹ independently represents —N(H)C(O)CH₃, —N(H)C(O)CF₃, —OCH₃, —N(H)S(O)₂CH₃ or —N(H)S(O)₂CF₃; and/or
each G² and/or G³ (e.g. G²) independently represents F, Cl, —N(H)C(O)CH₃, —N(H)C(O)CF₃, —OCH₃, —N(H)S(O)₂CH₃, —N(Bn)S(O)₂CH₃ or —N(H)S(O)₂CF₃.

In certain embodiments:
Z represents phenyl optionally substituted by one or more (e.g. one or two, such as one) groups independently selected from G²; and/or (e.g. and) each G² independently represents —N(Bn)S(O)₂CH₃.

For the avoidance of doubt, the skilled person will understand that Bn refers to a benzyl (i.e. —CH₂Ph) group.

In yet more particular embodiments:
each G¹ independently represents-OCH₃; and/or
each G² and/or G³ (e.g. G²) independently represents F, Cl or —OCH₃.

In particular embodiments, where Z represents
(i) cyclohexyl, optionally substituted by one or more (e.g. one or two, such as one) groups independently selected from G¹, or
(ii) phenyl optionally substituted by one or more (e.g. one or two, such as one) groups independently selected from G²,
one G¹ or G² group, as appropriate, is present and is located in the 4-position relative to the point of attachment.

Thus, in particular embodiments, Z may instead be represented as one of the following groups:

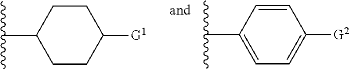

wherein G¹ and G² are as defined herein.

In alternative embodiments, where Z represents phenyl optionally substituted by one or more (e.g. one or two, such as one) groups independently selected from G², one G¹ or G² group, as appropriate, is present and is located in the 2- or 3-position relative to the point of attachment.

The skilled person will understand that, where Z represents cyclohexyl substituted by at least one (e.g. one) G¹, such as wherein the G¹ is in the 4-position relative to the point of attachment to the remainder of the molecule, the bond forming the point of attachment to the remainder of the molecule and the bond to the G¹ group may be in the cis or trans configurations.

In particular embodiments, where Z represents cyclohexyl substituted by at least one (e.g. one) G¹, such as wherein the G¹ is in the 4-position relative to the point of attachment to the remainder of the molecule, the bond forming the point of attachment to the remainder of the molecule and the bond to the G¹ group is in the trans configuration, such as wherein the carbon atoms of the cyclohexyl at those positions are in the (R) and(S) configurations respectively.

Thus, in particular embodiments, where Z represents cyclohexyl substituted by one G¹, that Z group may be depicted as follows:

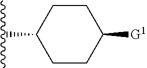

wherein G¹ is as defined herein.

In particular embodiments:
p represents 0;
q represents 2; and/or
t represents 2.

In particular embodiments, the compound of formula I is not:
(S)-(4-((4-chlorophenoxy)methyl)-7-azabicyclo[2.2.1]heptan-1-yl)(3-fluorophenyl)methanol; or
(R)-(4-((4-chlorophenoxy)methyl)-7-azabicyclo[2.2.1]heptan-1-yl) (3-fluorophenyl)methanol.

As described herein, compounds of the first aspect of the invention may also contain one or more asymmetric carbon atoms and may therefore exhibit optical and/or diastereoisomerism. Moreover, it has been found that certain such optical and/or diastereoisomers may show increased utility in the treatment of conditions as described herein, e.g. hyperglycemia or disorders characterized by hyperglycemia (such as type 2 diabetes).

The skilled person will understand that the compound of formula I may be such that the carbon substituted with the essential-OH group is in the (R) configuration or the(S) configuration, as understood by those skilled in the art.

For example, the compound of formula I may be a compound of formulae IA or IB

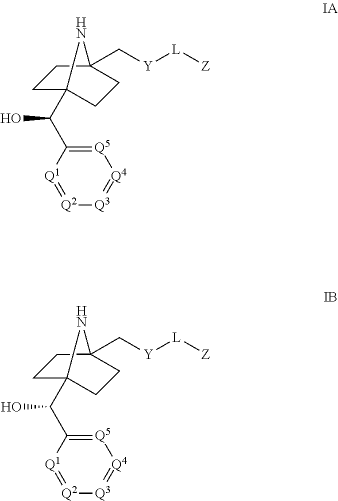

wherein the ring comprising Q¹ to Q⁵, and Y, L and Z are as described herein (i.e. as described in the first aspect of the invention, including all embodiments and particular features, and combinations thereof).

In particular embodiments, the compound of formula I is a compound of formula IA.

The skilled person will understand that, in addition to the carbon bearing the essential hydroxy group, compounds of the invention may comprise further sterocentres. For the aviodance of doubt, unless specified, the stereochemistry at all stereocentres (including stereochemistry present in positions other than the carbon bearing the essential hydroxy group) may be in either configuration (i.e. in the R or S configuration), or may be present in compounds as a mixture thereof (e.g. a racemic mixture).

Particular compounds of the first aspect of the invention that may be mentioned include the compounds of the examples provided herein, and pharmaceutically acceptable salts thereof.

More particular such compounds of the invention that may be mentioned include:
(R)-(4-(((4-Chlorobenzyl)oxy)methyl)-7-azabicyclo[2.2.1]heptan-1-yl)(3-fluorophenyl)-methanol;
(S)-(4-(((4-Chlorobenzyl)oxy)methyl)-7-azabicyclo[2.2.1]heptan-1-yl)(3-fluorophenyl)-methanol;
(R)-(4-(((4-Fluorobenzyl)oxy)methyl)-7-azabicyclo[2.2.1]heptan-1-yl)(3-fluorophenyl)-methanol;
(S)-(4-(((4-Fluorobenzyl)oxy)methyl)-7-azabicyclo[2.2.1]heptan-1-yl)(3-fluorophenyl)-methanol;
(R)-(4-(((4-Fluorobenzyl)oxy)methyl)-7-azabicyclo[2.2.1]heptan-1-yl)(5-fluoropyridin-3-yl)methanol;
(S)-(4-(((4-Fluorobenzyl)oxy)methyl)-7-azabicyclo[2.2.1]heptan-1-yl)(5-fluoropyridin-3-yl)methanol;
(R)-(4-Benzyl-7-azabicyclo[2.2.1]heptan-1-yl)(3-fluorophenyl)methanol;
(S)-(4-Benzyl-7-azabicyclo[2.2.1]heptan-1-yl)(3-fluorophenyl)methanol;
(R)-(3-Fluorophenyl)(4-(4-methoxybenzyl)-7-azabicyclo[2.2.1]heptan-1-yl)methanol;
(S)-(3-Fluorophenyl)(4-(4-methoxybenzyl)-7-azabicyclo[2.2.1]heptan-1-yl)methanol;
(R)-(4-(4-Fluorobenzyl)-7-azabicyclo[2.2.1]heptan-1-yl)-(3-fluorophenyl)methanol;
(S)-(4-(4-Fluorobenzyl)-7-azabicyclo[2.2.1]heptan-1-yl)-(3-fluorophenyl)methanol;
(R)-(3-Fluorophenyl)(4-(2-(trans-4-methoxycyclohexyl)ethyl)-7-azabicyclo[2.2.1]heptan-1-yl)methanol; and
(S)-(3-Fluorophenyl)(4-(2-((trans)-4-methoxycyclohexyl)ethyl)-7-aza-bicyclo[2.2.1]heptan-1-yl)methanol,
   and pharmaceutically acceptable salts thereof.

Further such compounds of the invention that may be mentioned include:
(R)-(3-Fluorophenyl)(4-(((4-(trifluoromethoxy)benzyl)oxy)methyl)-7-azabicyclo[2.2.1]heptan-1-yl)methanol;
(S)-(3-Fluorophenyl)((4-(((4-(trifluoromethoxy)benzyl)oxy)methyl)-7-azabicyclo[2.2.1]heptan-1-yl)methanol;
(R)-(5-Fluoropyridin-3-yl)(4-(((4-(trifluoromethoxy)benzyl)oxy)-methyl)-7-azabicyclo[2.2.1]heptan-1-yl)methanol;
(S)-(5-Fluoropyridin-3-yl)(4-(((4-(trifluoromethoxy)benzyl)oxy)methyl)-7-azabicyclo[2.2.1]heptan-1-yl)methanol;
(R)-(3-Fluorophenyl)(4-(4-(trifluoromethoxy)benzyl)-7-azabicyclo[2.2.1]heptan-1-yl)methanol;
(S)-(3-Fluorophenyl)(4-(4-(trifluoromethoxy)benzyl)-7-azabicyclo[2.2.1]heptan-1-yl)methanol;
N-Benzyl-N-(4-(4-((R)-(3-fluorophenyl)(hydroxy)methyl)-7-azabicyclo[2.2.1]heptan-1-yl)methyl)phenyl)methanesulfonamide;
N-Benzyl-N-(4-((4-((S)-(3-fluorophenyl)(hydroxy)methyl)-7-azabicyclo[2.2.1]heptan-1-yl)methyl)phenyl)methanesulfonamide;
N-Benzyl-N-(3-((4-((R)-(3-fluorophenyl)(hydroxy)methyl)-7-azabicyclo[2.2.1]heptan-1-yl)methyl)phenyl)methanesulfonamide;
N-Benzyl-N-(3-((4-((S)-(3-fluorophenyl)(hydroxy)methyl)-7-azabicyclo[2.2.1]heptan-1-yl)methyl)phenyl)methanesulfonamide;
N-(4-((4-((R)-(3-Fluorophenyl)(hydroxy)methyl)-7-azabicyclo[2.2.1]heptan-1-yl)methyl)phenyl)methanesulfonamide;
N-(4-((4-((S)-(3-Fluorophenyl)(hydroxy)methyl)-7-azabicyclo[2.2.1]heptan-1-yl)methyl)phenyl)methanesulfonamide;
N-(3-((4-((R)-(3-Fluorophenyl)(hydroxy)methyl)-7-azabicyclo[2.2.1]heptan-1-yl)methyl)phenyl)methanesulfonamide;
N-(3-((4-((S)-(3-Fluorophenyl)(hydroxy)methyl)-7-azabicyclo[2.2.1]heptan-1-yl)methyl)phenyl)methanesulfonamide;
(R)-(4-Cinnamyl-7-azabicyclo[2.2.1]heptan-1-yl)(5-fluoropyridin-3-yl)methanol;
(S)-(4-Cinnamyl-7-azabicyclo[2.2.1]heptan-1-yl)(5-fluoropyridin-3-yl)methanol;
(R)-(4-Cinnamyl-7-azabicyclo[2.2.1]heptan-1-yl)(3-fluorophenyl) methano;
(S)-(4-Cinnamyl-7-azabicyclo[2.2.1]heptan-1-yl)(3-fluorophenyl)methanol;
(R)-(5-Fluoropyridin-3-yl)(4-(3-phenylpropyl)-7-azabicyclo[2.2.1]heptan-1-yl)methanol;
(S)-(5-Fluoropyridin-3-yl)(4-(3-phenylpropyl)-7-azabicyclo[2.2.1]heptan-1-yl)methanol;
(R)-(3-Fluorophenyl)(4-(3-phenylpropyl)-7-azabicyclo[2.2.1]heptan-1-yl)methanol;
(S)-(3-Fluorophenyl)(4-(3-phenylpropyl)-7-azabicyclo[2.2.1]heptan-1-yl)methanol;
(R)-4-(4-Chlorophenethyl)-7-azabicyclo[2.2.1]heptan-1-yl)(3-fluorophenyl)methanol;
(S)-(4-(4-Chlorophenethyl)-7-azabicyclo[2.2.1]heptan-1-yl)(3-fluorophenyl)methanol;
(R)-(3-Fluorophenyl)(4-(4-methoxyphenethyl)-7-azabicyclo[2.2.1]heptan-1-yl)methanol;
(S)-(3-Fluorophenyl)(4-(4-methoxyphenethyl)-7-azabicyclo[2.2.1]heptan-1-yl)methanol;
(R)-(5-Fluoropyridin-3-yl)(4-(4-methoxyphenethyl)-7-azabicyclo[2.2.1]heptan-1-yl)methanol;
(S)-(5-Fluoropyridin-3-yl)(4-(4-methoxyphenethyl)-7-azabicyclo[2.2.1]heptan-1-yl)methanol;
(R)-(3-Fluorophenyl)(4-(4-(trifluoromethoxy) phenethyl)-7-azabicyclo[2.2.1]heptan-1-yl)methanol;
(S)-(3-Fluorophenyl)(4-(4-(trifluoromethoxy) phenethyl)-7-azabicyclo[2.2.1]heptan-1-yl)methanol
(R)-(5-Fluoropyridin-3-yl)(4-(4-(trifluoromethoxy) phenethyl)-7-azabicyclo[2.2.1]heptan-1-yl)methanol;
(S)-(5-Fluoropyridin-3-yl)(4-(4-(trifluoromethoxy) phenethyl)-7-azabicyclo[2.2.1]heptan-1-yl)methanol;
(R)-(2-Fluorophenyl)(4-(2-(pyridin-3-yl)ethyl)-7-azabicyclo[2.2.1]heptan-1-yl)methanol;
(S)-(2-Fluorophenyl)(4-(2-(pyridin-3-yl)ethyl)-7-azabicyclo[2.2.1]heptan-1-yl)methanol;
(R)-(3-Fluorophenyl)(4-(2-(pyridin-3-yl)ethyl)-7-azabicyclo[2.2.1]heptan-1-yl)methanol;
(S)-(3-Fluorophenyl)(4-(2-(pyridin-3-yl)ethyl)-7-azabicyclo[2.2.1]heptan-1-yl)methanol,
   and pharmaceutically acceptable salts thereof.

The skilled person will understand that references to specific stereoisomer(s) of a compound of formula I, will refer to the specific stereoisomer present in the substantial absence of the other (corresponding) stereoisomer(s) (e.g. in the case of compounds of formula I, where the carbon substituted by the essential-OH group is in the R configuration, the substantial absence of corresponding compounds wherein the carbon substituted by the essential-OH group is in the opposite configuration, i.e. the S configuration).

As used herein, references to the substantial absence of the corresponding opposite stereoisomer may refer to the desired stereoisomer being present at a purity of at least 80% (e.g. at least 90%, such as at least 95%) relative to the opposite stereoisomer. Alternatively, in such instances, compounds may be indicated to be present in the substantial absence of the compound in the other configuration, which may indicate that the compound in the relevant configuration is present in an enantiomeric excess (e.e.), or when two or more stereogenic centres are defined, in a diastereomeric excess (d.e.), of at least 90% (such as at least 95%, at least 98% or, particularly, at least 99%, for example at least 99.9%).

For the avoidance of doubt, where the stereochemistry of more than one position is specified, the compound will be present in the substantial absence of the other diastereoisomers.

For the avoidance of doubt, where the sterochemistry of a particular position is not specified, compounds of the invention will include compounds wherein that position has either available sterochemical configuration, and mixtures (e.g. racemic mixtures) thereof. Thus, compounds referred to as having a specific stereochemistry at a defined position (e.g. in the case of compounds of formula I, at the carbon substituted by the essential —OH) may also have stereochemistry at one or more other positions, and so may exist as mixtures of enantiomers or diastereoisomers in relation to the stereochemistry at those positions.

### Medical Uses

As indicated herein, the compounds of the invention, and therefore compositions and kits comprising the same, are useful as pharmaceuticals.

Thus, according to a second aspect of the invention there is provided a compound of the first aspect of the invention, as hereinbefore defined (i.e. a compound as defined in the first aspect of the invention, including all embodiments and particular features thereof), for use in medicine (i.e. for use as a pharmaceutical, which may be described as use as a medicament).

Compounds described herein are β₂ adrenergic receptor agonists and therefore suitable in treating diseases such as those described herein. Such activity may be observed in compounds of the invention by identifying compounds which stimulate the uptake of glucose in skeletal muscle cells, which activity may be confirmed to be mediated by activation of the β₂ receptor by observation that such activity is prevented or diminished in the presence of a (e.g. selective) β₂ adrenergic receptor antagonist (such in the biological example provided herein).

Thus, in a third aspect of the invention, there is provided a compound of the first aspect of the invention, as hereinbefore defined, for use in treating a disease or disorder the treatment of which is mediated by activation of the β₂ adrenergic receptor.

In an alternative third aspect of the invention, there is provided the use of a compound of the first aspect of the invention in the manufacture of a medicament for use in treating a disease or disorder the treatment of which is mediated by activation of the β₂ adrenergic receptor.

In a further alternative third aspect of the invention, there is provided a method of treating a disease or disorder the treatment of which is mediated by activation of the β₂ adrenergic receptor comprising administering to a patient in need thereof a therapeutically effective amount of a compound of the first aspect of the invention.

For the avoidance of doubt, references to compounds as defined in the first aspect of the invention will include references to compounds of formula I (including all embodiments thereof) and pharmaceutically acceptable salts thereof.

As indicated herein, the compounds of the invention act by inducing uptake of glucose in skeletal muscle cells, thus allowing for the reduction of blood glucose levels in vivo. Thus, compounds of the invention may be of particular use in treating hyperglycemia or a disorder characterized by hyperglycaemia.

In a particular embodiment of the third aspect of the invention, there is provided a compound of the first aspect of the invention, as hereinbefore defined, for use in the treatment of hyperglycemia or a disorder characterized by hyperglycaemia.

In an alternative embodiment of the third aspect of the invention, there is provided the use of a compound of the first aspect of the invention in the manufacture of a medicament for use in the treatment of hyperglycaemia or a disorder characterized by hyperglycaemia.

In a further alternative embodiment of the third aspect of the invention, there is provided a method of treating hyperglycemia or a disorder characterized by hyperglycemia comprising administering to a patient in need thereof a therapeutically effective amount of a compound of the first aspect of the invention.

For the avoidance of doubt, the term “hyperglycemia” as used herein will be understood by those skilled in the art to refer to a condition wherein an excessive amount of glucose circulates in blood plasma of the subject experiencing the same. In particular, it may refer to a subject (e.g a human subject) having blood glucose levels higher than about 10.0 mmol/L (such as higher than about 11.1 mmol/L, e.g. higher than about 15 mmol/L), although it may also refer to a subject (e.g. a human subject) having blood glucose levels higher than about 7 mmol/L for an extended period of time (e.g. for greater than 24 hours, such as for greater than 48 hours).

The skilled person will understand that references to the treatment of a particular condition (or, similarly, to treating that condition) take their normal meanings in the field of medicine. In particular, the terms may refer to achieving a reduction in the severity of one or more clinical symptom associated with the condition. For example, in the case of type 2 diabetes, the term may refer to achieving a reduction of blood glucose levels. In particular embodiments, in the case of treating hyperglycemia or conditions characterised by hyperglycaemia, the term may refer to achieving a reduction of blood glucose levels (for example, to or below about 10.0 mmol/mL (e.g. to levels in the range of from about 4.0 mmol/L to about 10.0 mmol/L), such as to or below about 7.5 mmol/mL (e.g. to levels in the range of from about 4.0 mmol/L to about 7.5 mmol/L) or to or below about 6 mmol/mL (e.g. to levels in the range of from about 4.0 mmol/L to about 6.0 mmol/L)).

As used herein, references to patients will refer to a living subject being treated, including mammalian (e.g. human) patients. Thus, in particular embodiments of the first aspect of the invention, the treatment is in a mammal (e.g. a human).

As used herein, the term therapeutically effective amount will refer to an amount of a compound that confers a therapeutic effect on the treated patient. The effect may be objective (i.e. measurable by some test or marker) or subjective (i.e. the subject gives an indication of and/or feels an effect).

Although compounds of the first aspect of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the first aspect of the invention are included within the scope of the invention.

For the avoidance of doubt, the compounds of the first aspect of the invention are useful because they possess pharmacological activity, and/or are metabolised in the body following oral or parenteral administration to form compounds that possess pharmacological activity. In particular, as described herein, compounds of the first aspect of the invention are useful in the treatment of hyperglycaemia or disorders characterized by hyperglycemia (such as type 2 diabetes), which terms will be readily understood by one of skill in the art (as described herein).

In a particular embodiment, the treatment is of a disorder (which may also be referred to as a condition or disease) characterised by hyperglycemia.

In particular embodiments of the first aspect of the invention, the disorder is type 2 diabetes, such as type 2 diabetes of a sub-type selected from the list consisting of maturity-onset diabetes in the young (MODY), ketosis-prone diabetes in adults, latent autoimmune diabetes of adults (LADA), and gestational diabetes.

In further embodiments, the disorder is type 1 diabetes, particularly wherein the treatment further comprises treatment with insulin (or a derivative and/or functional mimetic thereof).

In particular embodiments, compounds of the invention (i.e. compounds of formula I, including all embodiments thereof) are for use in the treatment of type 2 diabetes (or useful in the manufacture of a medicament for such treatment, or useful in a method for such treatment, as described herein).

In further particular embodiments, the treatment of type 2 diabetes is in a non-obese patient.

For the avoidance of doubt, the skilled person will understand that patients with a Body Mass Index (BMI) of greater than 30 are considered to be obese.

In particular embodiments, the treatment may be of hyperglycemia in a patent who is at risk of developing type 2 diabetes, which condition may be defined as pre-diabetes. Thus, compounds of the invention may be useful in the prevention of type 2 diabetes (e.g. in a patient having pre-diabetes).

As used herein, the term prevention (and, similarly, preventing) includes references to the prophylaxis of the disease or disorder (and vice-versa). As such, references to prevention may also be references to prophylaxis, and vice versa. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

In more particular embodiments, the type 2 diabetes is characterised by the patient displaying severe insulin resistance (SIR).

In further embodiments, the treatment may be of hyperglycemia in a patient having type 1 diabetes. Thus, compounds of the invention may be useful in the treatment of hyperglycemia in type 1 diabetes.

The skilled person will understand that compounds of the invention may be useful in treating hyperglycemia in patients having impaired insulin production, such as in patients having cystic fibrosis. Thus, in further embodiments, the disorder characterized by hyperglycemia is cystic fibrosis-related diabetes.

In particular embodiments that may be mentioned, the disorder characterised by hyperglycemia is (or is characterized by) severe insulin resistance (SIR), which may be understood by those in the art to refer to disorders wherein typically the subject has normal, or in some cases increased, insulin production but significantly reduced insulin sensitivity. In particular instances, such patients may be non-obese (e.g. being of a healthy weight). Thus, in particular embodiments, such treatments are performed in patients who are not defined as being obese (e.g. in patients who are defined as being of a healthy weight).

For example, SIR may be identified in a patient based in said patient having fasting insulin >150 pmol/L and/or a peak insulin on glucose tolerance testing of >1,500 pmol/L, particularly in individuals with a BMI <30 kg/m² (which patient may otherwise have normal glucose tolerance).

More particularly, SIR may be characterised by the patient having no significant response to the presence of insulin, which may result from a defect (e.g. a genetic defect) in the function of the insulin receptor.

Particular disorders that may be characterised by SIR include: Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

More particular disorders that may be characterised by SIR include Donohue's syndrome and Type A syndrome of insulin resistance and, yet more particularly, Rabson-Mendenhall syndrome.

The skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of type 2 diabetes, such as treatment with one or more other therapeutic agent that is useful in the treatment of type 2 diabetes as known to those skilled in the art, such as therapies comprising requiring the patient to undergo a change of diet and/or undertake exercise regiments, and/or surgical procedures designed to promote weight loss (such as gastric band surgery).

In particular, treatment with compounds of the invention may be performed in combination with (e.g. in a patient who is also being treated with) one or more (e.g. one) additional compounds (i.e. therapeutic agents) that:
(i) are capable of reducing blood sugar levels; and/or
(ii) are insulin sensitizers; and/or
(iii) enhance insulin release,
   all of which are described herein below.

In alternative embodiments, compounds of the first aspect of the invention (i.e. compounds of the invention) may be useful in the treatment of a non-alcoholic fatty liver disease (NAFLD).

Non-alcoholic fatty liver disease (NAFLD) is defined by excessive fat accumulation in the form of triglycerides (steatosis) in the liver (designated as an accumulation of greater than 5% of hepatocytes histologically). It is the most common liver disorder in developed countries (for example, affecting around 30% of US adults) and most patients are asymptomatic. If left untreated, the condition may progressively worsen and may ultimately lead to cirrhosis of the liver. NAFLD is particularly prevalent in obese patents, with around 80% thought to have the disease.

A sub-group of NAFLD patients (for example, between 2 and 5% of US adults) exhibit liver cell injury and inflammation in addition to excessive fat accumulation. This condition, designated as non-alcoholic steatohepatitis (NASH), is virtually indistinguishable histologically from alcoholic steatohepatitis. While the simple steatosis seen in NAFLD does not directly correlate with increased short-term morbidity or mortality, progression of this condition to NASH dramatically increases the risks of cirrhosis, liver failure and hepatocellular carcinoma. Indeed, NASH is now considered to be one of the main causes of cirrhosis (including cryptogenic cirrhosis) in the developed world.

The exact cause of NASH has yet to be elucidated, and it is almost certainly not the same in every patient. It is most closely related to insulin resistance, obesity, and the metabolic syndrome (which includes diseases related to diabetes mellitus type 2, insulin resistance, central (truncal) obesity, hyperlipidaemia, low high-density lipoprotein (HDL) cholesterol, hypertriglyceridemia, and hypertension). However, not all patients with these conditions have NASH, and not all patients with NASH suffer from one of these conditions. Nevertheless, given that NASH is a potentially fatal condition, leading to cirrhosis, liver failure and hepatocellular carcinoma, there exists a clear need for an effective treatment.

In particular embodiments, compounds of the invention (i.e. compounds of formula I, including all embodiments thereof) are for use in the treatment of a non-alcoholic fatty liver disease (or useful in the manufacture of a medicament for such treatment, or useful in a method for such treatment, as described herein).

The process by which the triglyceride fat accumulates in liver cells is called steatosis (i.e. hepatic steatosis). The skilled person will understand that the term “steatosis” encompasses the abnormal retention of fat (i.e. lipids) within a cell. Thus, in particular embodiments of the first aspect of the invention, the treatment or prevention is of a fatty liver disease which is characterized by steatosis.

During steatosis, excess lipids accumulate in vesicles that displace the cytoplasm of the cell. Over time, the vesicles can grow large enough to distort the nucleus, and the condition is known as macrovesicular steatosis. Otherwise, the condition may be referred to as microvesicular steatosis. Steatosis is largely harmless in mild cases; however, large accumulations of fat in the liver can cause significant health issues. Risk factors associated with steatosis include diabetes mellitus, protein malnutrition, hypertension, obesity, anoxia, sleep apnea and the presence of toxins within the cell.

As described herein, fatty liver disease is most commonly associated with alcohol or a metabolic syndrome (for example, diabetes, hypertension, obesity or dyslipidemia). Therefore, depending on the underlying cause, fatty liver disease may be diagnosed as alcohol-related fatty liver disease or non-alcoholic fatty liver disease (NAFLD).

Particular diseases or conditions that are associated with fatty liver disease that are not related to alcohol include metabolic conditions such as diabetes, hypertension, obesity, dyslipidemia, abetalipoproteinemia, glycogen storage diseases, Weber-Christian disease, acute fatty liver of pregnancy, and lipodystrophy. Other non-alcohol related factors related to fatty liver diseases include malnutrition, total parenteral nutrition, severe weight loss, refeeding syndrome, jejunoileal bypass, gastric bypass, polycystic ovary syndrome and diverticulosis.

The compounds of the invention have been found to be particularly useful in the treatment or prevention of NAFLD, which may be referred to as a fatty liver disease which is not alcohol related. A fatty liver disease which is “not alcohol related” may be diagnosed wherein alcohol consumption of the patient is not considered to be a main causative factor. A typical threshold for diagnosing a fatty liver disease as “not alcohol related” is a daily consumption of less than 20 g for female subjects and less than 30 g for male subjects.

If left untreated, subjects suffering from fatty liver disease may begin to experience inflammation of the liver (hepatitis). It has been postulated that one of the possible causes of this inflammation may be lipid peroxidative damage to the membranes of the liver cells. Inflammation of a fatty liver can lead to a number of serious conditions and it is therefore desirable to treat or prevent fatty liver disease before inflammation occurs. Thus, in particular embodiments of the first aspect of the invention, the treatment or prevention is of a NAFLD which is associated with inflammation.

Non-alcoholic steatohepatitis (NASH) is the most aggressive form of NAFLD, and is a condition in which excessive fat accumulation (steatosis) is accompanied by inflammation of the liver. If advanced, NASH can lead to the development of scar tissue in the liver (fibrosis) and, eventiually, cirrhosis. As described above, the compounds of the invention have been found to be useful in the treatment or prevention of NAFLD, particularly when accompanied by inflammation of the liver. It follows that the compounds of the invention are also useful in the treatment or prevention of NASH. Therefore, in a further embodiment of the first aspect of the invention, the treatment or prevention is of non-alcoholic steatohepatitis (NASH).

The skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of a fatty liver disease, as described herein, such as treatment with one or more other therapeutic agent that is useful in the treatment of a fatty liver disease as known to those skilled in the art; for example, therapies comprising requiring the patient to undergo a change of diet and/or undertake exercise regiments, and/or surgical procedures designed to promote weight loss (such as gastric band surgery).

In particular, treatment with compounds of the invention may be performed in combination with (e.g. in a patient who is also being treated with) one or more (e.g. one) additional compounds (i.e. therapeutic agents) that are capable of reducing the level of fat (e.g. triglycerides) in the liver.

References to treatment of a fatty liver disease may refer to achieving a therapeutically significant reduction of fat (e.g. triglycerides levels) in liver cells (such as a reduction of at least 5% by weight, e.g. a reduction of at least 10%, or at least 20% or even 25%).

As described herein, compounds of the invention may be of use in treating a disease or disorder the treatment of which is mediated by activation of the β₂ adrenergic receptor.

In particular embodiments, the compounds of the first aspect of the invention may be understood to positively modulate the β₂ adrenergic receptor, which compounds may be referred to as a β₂-adrenergic receptor agonist.

The skilled person will appreciate what is meant by “β₂ adrenergic receptor” (or “β₂-AR”). Such receptors are known in the art and have been reviewed in, e.g., Johnson. M., J. Allergy Clin. Immunol., 117, 18-24 (2006). For the avoidance of doubt, adrenergic receptors are a class of G protein-coupled receptors which bind and are activated by their endogenous ligands, the catecholamines, adrenaline and noradrenaline. The adrenergic receptor falls into five types: α₁, α₂, β₁, β₂ and β₃. These subtypes are expressed in distinct patterns and involved in different physiological processes, such that ligands that can selectively target one subtype have therapeutic potential for multiple diseases. The present invention is concerned with the β₂ adrenergic receptor, although compounds may interact with one or more other adrenergic receptor (e.g. one or more other β adrenergic receptor).

The term “positively modulates β₂-adrenergic receptor activity” will be understood to mean that the compound is capable of altering the signalling of the receptor.

As used herein, the term “β₂ agonist” is used to mean β₂ adrenergic receptor agonist. In certain embodiments, the term β₂ agonist is understood to include compounds that are primarily β₂ agonists, but may also exhibit some agonism for other adrenergic receptors. In this application, the terms “β₂ adrenergic receptor agonist”, “β₂ AR agonist”, “β₂AR agonist” and “β₂ agonist” may be used interchangeably.

Thus, in certain embodiments, references to β₂ agonists may include both selective and non-selective agonists.

In certain embodiments, references to β₂ agonists may include any ligand that change receptor signalling including but not limited to full and partial agonists. Further, β₂ agonists that may be used in accordance with various aspects and embodiments of the present disclosure may be short-acting, long acting or ultra long-acting.

As used herein, the term “mediated by activation of the β₂ adrenergic receptor” is used to indicate that activation of the receptor regulates or causes a physiological response which will in turn provide a biological effect corresponding to (or leading to) treatment of the disease or disorder.

As used in herein, references to diseases and disorders the treatment of which is “mediated by activation of the β₂ adrenergic receptor” may also refer to diseases and disorders (and in particular the treatment thereof) being, inter alia, “associated with”, “mediated by”, “affected by”, “regulated by”, “modulated by” and “linked to” the β₂ adrenergic receptor.

As described herein, diseases and disorders the treatment of which is mediated by activation of the β₂ adrenergic receptor will be known to those skilled in the art. Thus, the skilled person will understand that in respect of certain of the diseases and disorders described herein the suitability of compounds of the invention for the treatment of such diseases and disorders may be known to those skilled in the art; for example, based on the disclosures referred to herein below (the contents of which are incorporated herein by reference).

In addition to those as may be described herein above, particular diseases and disorders the treatment of which is mediated by activation of the β₂ adrenergic receptor that may be mentioned include:
neurodegenerative diseases, such as MCI (mild cognitive impairment), aMCI (amnestic MCI), vascular dementia, mixed dementia, FTD (front-temporal dementia), HD (Huntington disease), Rett syndrome, PSP (progressive supranuclear palsy), CBD (corticobasal degeneration), SCA (spinocerebellar ataxia), MSA (multiple system atrophy), SDS (Shy-Drager syndrome), olivopontocerebellar atrophy, TBI (traumatic brain injury), CTE (chronic traumatic encephalopathy), stroke, EKS (Wernicke-Korsakoff syndrome), normal pressure hydrocephalus, hypersomnia (narcolepsy), ASD (autistic spectrum disorders), FXS (fragile X syndrome), YSC (tubular sclerosis complex), prion-related disorders, CJD (Creutzfeldt-Jakob disease), depressive disorders, DLC (dementia with Lewy bodies), PD (Parkinson's disease), PDD (PD dementia), ADHD (attention deficit hyperactivity disorder), Alzheimer's disease (AD), early AD and DS (Down syndrome);
muscle dystrophy or a disorder characterised by muscular dystrophy, such as muscle damage, muscle wasting, muscle atrophy, muscle degeneration or sclerosis;
kidney disease, such as CKD (chronic kidney disease), ESRD (end-stage renal disease) and diabetic nephropathy;
inflammation or a disorder characterised by inflammation, such as sepsis, psoriasis, dermatitis, psoriasis-like skin dermatitis, lacerations or HDF (human dermal fibroblasts), and including localised acute inflammation, such as that related to endotoxemia and Acute Lung Injury (ALI), and respiratory conditions associated with inflammation, such as asthma and other pulmonary disorders, such as chronic obstructive pulmonary disease (COPD); and
an autoimmune disease, such as SLE (systemic lupus erythematosus, RA (rheumatoid arthritis), MG (myasthenia gravis) MS and GD (Grave's disease).

The suitability of β₂ adrenergic receptor agonists for treating such conditions may be demonstrated by the data provided herein and by reference to the literature known to those skilled on the art, such as that described herein (the whole contents of which, in particular the experimental results presented, will be understood to be incorporated herein by reference).

In particular, the suitability of β₂ adrenergic receptor agonists for treating certain of the diseases and disorders referred to herein may be identified in and, in some instances, confirmed by the disclosures of WO 2020/198466 A1 and WO 2021/003161 A1 (which, for the avoidance of doubt, are incorporated herein by reference, in particular the examples as provided therein).

In a particular embodiment, there is provided a compound of the first aspect of the invention, as hereinbefore defined, for use in treating neurodegenerative diseases.

In particular embodiments, the neurodegenerative disease is selected from MCI (mild cognitive impairment), aMCI (amnestic MCI), vascular dementia, mixed dementia, FTD (front-temporal dementia), HD (Huntington disease), Rett syndrome, PSP (progressive supranuclear palsy), CBD (corticobasal degeneration), SCA (spinocerebellar ataxia), MSA (multiple system atrophy), SDS (Shy-Drager syndrome), olivopontocerebellar atrophy, TBI (traumatic brain injury), CTE (chronic traumatic encephalopathy), stroke, EKS (Wernicke-Korsakoff syndrome), normal pressure hydrocephalus, hypersomnia (narcolepsy), ASD (autistic spectrum disorders), FXS (fragile X syndrome), YSC (tubular sclerosis complex), prion-related disorders, CJD (Creutzfeldt-Jakob disease), depressive disorders, DLC (dementia with Lewy bodies), PD (Parkinson's disease), PDD (PD dementia), ADHD (attention deficit hyperactivity disorder), Alzheimer's disease (AD), early AD and DS (Down syndrome).

Mittal. S., et al., Science., 357(6354), 891-898 (2017) describes that β2-adrenergic receptor agonists promote dopamine neuron health by reducing SNCA expression through H2K27 deacetylation and mitochondrial free radicals. This may benefit nigral dopamine neurons, which are prone to mitochondrial bioenergetics dysfunction at early stages of Lewy body neuropathy. β₂-adrenergic receptor agonists are expressed in the substantia nigra and cortex, regions that are progressively affected by Parkinson's disease (PD). Therefore, β₂-adrenergic receptor agonists can be used to reduce the risk and affect of PD.

Hishida. R., The Lancet, 870 (1992) describes that β₂-adrenergic receptor agonists can beneficially affect wearing-off in patients with Parkinson's disease on long-term levodopa.

Uc, E. Y., et al., Clin. Neuropharmacol., 26 (4), 207-212 (2003) describes that β₂-adrenergic receptor agonist, albuterol, benefited patients with PD through two mechanisms, an increased response to levodopa and an increase in muscle mass.

O'Neill, et al., Br. J. Pharmacol., 177, 282-297 (2019) describes that β₂-adrenergic receptor agonists restrict microglial activation and protect against the onset and progression of dopamine neuronal cell loss and related motor deficits provoke by central or systemic inflammation. Therefore, targeting β₂-adrenergic receptors with a β₂-adrenergic receptor agonist imbues an intervening prophylactic mechanism to protect against the progression of neurodegeneration and exacerbated decline in motor function associated with systemic and central inflammation. As a result, β₂-adrenergic receptor agonists may be beneficial in the treatment of PD-related neuropathy and motor impairments induced by inflammation.

In alternative embodiments, there is provided a compound of the first aspect of the invention, as hereinbefore defined, for use in treating muscle dystrophy or a disorder characterised by muscular dystrophy.

In particular such embodiments, the muscle dystrophy is muscle damage, muscle wasting, muscle atrophy, muscle degeneration or sclerosis.

Jiang, G., et al., ISRN Pharma., 2011, 1-7 (2011) describes that β₂-AR agonists ameliorate animal wasting in denervation, amyotrophic lateral sclerosis, muscular dystrophy, disuse, aging and myocardial unloading models. Further, in patients with immobilization conditions or muscular dystrophy, β₂-AR agonists increase lean body mass and enhance skeletal muscle functions. Also, β₂-AR agonists were found to promote myocardial recovery in patients with myocardial unloading atrophy resulting from application of left ventricular assist devise.

Bartus, R. T., et al., Neurobiol. Dis., 85, 11-24, 2016 indicates that β2-adrenergic receptor agonists may enhance muscle bulk and muscle strength in amyotrophic lateral sclerosis (ALS) patients by increasing neurotrophic factors.

In alternative embodiments, there is provided a compound of the first aspect of the invention, as hereinbefore defined, for use in treating kidney disease.

In particular such embodiments, the kidney disease is selected from CKD (chronic kidney disease), ESRD (end-stage renal disease) and diabetic nephropathy.

Cleveland, K., et al., FASEB Journal, 33 (1), 514 (2019) describes that β2-adrenergic receptor agonists have been shown to induce mitochondrial biogenesis (MB) and promote recovery from acute kidney injury, and may find use as a potential therapy for diabetic nephropathy (DN).

Jesinkey, S. R., et al., J. Am. Soc. Nephrol., 25, 1157-1162 (2014) describes the necessity for mitochondrial biogenesis as an adaptive response for meeting the increased metabolic and energy demands during organ recovery after an acute injury. In particular, renal mitochondrial dysfunction has been linked to pathogenesis of acute kidney injury (AKI), a disorder characterised by a rapid decrease in kidney excretory function and subsequent retention of harmful waste products.

In alternative embodiments, there is provided a compound of the first aspect of the invention, as hereinbefore defined, for use in treating inflammation or a disorder characterised by inflammation.

In particular embodiments, the inflammation is (or is characterised by) sepsis, psoriasis, dermatitis, psoriasis-like skin dermatitis, lacerations or HDF (human dermal fibroblasts).

As the skilled person will know, inflammation is a tightly controlled process that ensures proper localization of immune cells, release of pro- and anti-inflammatory mediators, clearance of dead cells, and containment of the pathogen.

The skilled person will know that inflammation may also be a cause of respiratory conditions, such as asthma and other pulmonary disorders, such as chronic obstructive pulmonary disease (COPD).

Grailer, J. J. et al, J Innate Immun, 6, 607-618 (2014) shows that blockade of the β2 adrenergic receptor reduced survival and enhanced injury in mouse models of endotoxemia and LPS-induced acute lung injury, respectively. These results demonstrate the suitability of β₂AR activation in the treatment of localised acute inflammation, such as that related to endotoxemia and Acute Lung Injury.

Agac, D., et al., Brain, Behaviour and Immunity, 74, 176-185 (2018) describes that there is a unique synergistic pathway that converts acute inflammatory signals into an anti-inflammatory response and likely explains a variety of phenomena known to be involved in β₂-adrenergic receptor agonists mediated immune suppression. In particular, β2-adrenergic receptor agonists signalling directly controls anti-inflammatory cytokine, IL-10, expression. These results suggest the use of β₂AR agonists in the treatment of inflammatory disorders, such as sepsis.

Liu, F., et al., Cells, 511 (9), 1-17 (2020) describes that β2-adrenergic receptor agonists demonstrated significant anti-psoriasis effects, which may involve regulating the Th17/Tregs axis balances and glycerophospholipid metabolism in response to imiquimod (IMQ) induced psoriasis.

Provost, G. S., et al., J. Investig. Dermatol., 135, 279-288 (2015) describes that β2-adrenergic receptor agonists reduces human dermal fibroblast (HDF) differentiation, therefore reducing scarring to a patient following a laceration or open wound.

Wu, et al., Front. Pharmacol., 1313 (9), 1-9 (2018) teaches that β2-adrenergic receptor agonists may be a target treatment for autoimmune diseases (AD), such as SLE (systemic lupus erythematosus, RA (rheumatoid arthritis), MG (mysasthenia gravis) MS and GD (Grave's disease).

In alternative embodiments, there is provided a compound of the first aspect of the invention, as hereinbefore defined, for use in treating an autoimmune disease.

In particular such embodiments, the autoimmune disease is selected from SLE (systemic lupus erythematosus, RA (rheumatoid arthritis), MG (myasthenia gravis) MS and GD (Grave's disease).

### Pharmaceutical Compositions

As described herein, compounds of the first and, therefore, the second and third aspects of the invention are useful as pharmaceuticals. Such compounds may be administered alone or may be administered by way of known pharmaceutical compositions/formulations.

In a fourth aspect of the invention, there is provided a pharmaceutical composition comprising a compound as defined in the second or third aspect of the invention, and optionally one or more pharmaceutically acceptable adjuvant, diluent and/or carrier.

The skilled person will understand that references herein to compounds of the first aspect of the invention being for particular uses (and, similarly, to uses and methods of use relating to compounds of the invention) may also apply to pharmaceutical compositions comprising compounds of the invention as described herein.

In a fifth aspect of the invention, there is provided a pharmaceutical composition for use in the treatment of hyperglycemia or a disorder characterized by hyperglycemia (as defined herein, such as type 2 diabetes) comprising a compound as defined in the first aspect of the invention, and optionally one or more pharmaceutically acceptable adjuvant, diluent and/or carrier.

In an alternative fifth aspect of the invention, there is provided a pharmaceutical composition for use in the treatment or prevention of a non-alcoholic fatty liver disease, as defined herein.

In an alternative fifth aspect of the invention, there is provided a pharmaceutical composition for use in the treatment or prevention of a non-alcoholic fatty liver disease, as defined herein.

The skilled person will understand that compounds of the first (and, therefore, second and third) aspect of the invention may act systemically and/or locally (i.e. at a particular site).

The skilled person will understand that compounds and compositions as described in the first to fifth aspects of the invention will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form. Pharmaceutical compositions as described herein will include compositions in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration.

Thus, in particular embodiments of the fourth and fifth aspects of the invention, the pharmaceutical formulation is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The skilled person will understand that compounds of the invention, and pharmaceutically-acceptable salts thereof, may be administered (for example, as formulations as described hereinabove) at varying doses, with suitable doses being readily determined by one of skill in the art. Oral, pulmonary and topical dosages (and subcutaneous dosages, although these dosages may be relatively lower) may range from between about 0.01 μg/kg of body weight per day (μg/kg/day) to about 200 μg/kg/day, preferably about 0.01 to about 10 μg/kg/day, and more preferably about 0.1 to about 5.0 μg/kg/day. For example, when administered orally, treatment with such compounds may comprise administration of a formulations typically containing between about 0.01 μg to about 2000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 10 mg, 20 mg, 30 mg or 40 mg twice daily, or 10 μg, 20 μg, 30 μg or 40 μg twice daily).

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

As described herein above, the skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of hyperglycaemia or a disorder characterized by hyperglycemia (as defined herein, such as type 2 diabetes), such as treatment with one or more other therapeutic agent that is useful in the treatment of hyperglycemia or a disorder characterized by hyperglycemia (as defined herein, such as type 2 diabetes).

In particular embodiments of the fourth and fifth aspects of the invention, the pharmaceutical composition may further comprise one or more additional (i.e. other) therapeutic agent.

In more particular embodiments, the one or more additional therapeutic agent is an agent for the treatment of type 2 diabetes as known to those skilled in the art, such as metformin, sulfonylureas (e.g. carbutamide, acetohexamide, chlorpropamide, tolbutamide. glipizide (glucotrol), gliclazide, glibenclamide, glyburide (Micronase), glibornuride, gliquidone, glisoxepide, glyclopyramide, glimepiride (Amaryl), glimiprime, JB253 or JB558), thiazolidinediones (e.g. pioglitazone, rosiglitazone (Avandia), lobeglitazone (Duvie) and troglitazone (Rezulin)), dipeptidyl peptidase-4 inhibitors (e.g. sitagliptin, vildagliptin, saxagliptin, linagliptin, anagliptin, teneligliptin, alogliptin, trelagliptin, gemigliptin, dutogliptin and omarigliptin), SGLT2 inhibitors (e.g. dapagliflozin, empagliflozin, canagliflozin, ipragliflozin, tofogliflozin, sergliflozin etabonate, remogliflozin etabonate, and ertugliflozin), and glucagon-like peptide-1 (GLP-1) analogues.

The skilled person will understand that combinations of therapeutic agents may also described as a combination product and/or provided as a kit-of-parts.

In a sixth aspect of the invention, there is provided a combination product comprising:
(A) a compound as defined in the first aspect of the invention; and
(B) one or more additional therapeutic agent, wherein each of components (A) and (B) is formulated in admixture, optionally with one or more a pharmaceutically-acceptable adjuvant, diluent or carrier.

In a seventh aspect of the invention, there is provided a kit-of-parts comprising:
(a) a compound as defined in the first (or second and/or third) aspect of the invention, (or a pharmaceutical composition comprising the same) or a pharmaceutical composition as defined in the fourth or fifth aspect of the invention; and
(b) one or more other therapeutic agent, optionally in admixture with one or more pharmaceutically-acceptable adjuvant, diluent or carrier,
which components (a) and (b) are each provided in a form that is suitable for administration in conjunction with the other.

In particular embodiments (e.g. of the sixth and seventh aspects of the invention), the additional therapeutic agent is a therapeutic agent that is useful for the treatment of hyperglycemia or a disorder characterized by hyperglycemia (e.g. type 2 diabetes), as known to those skilled in the art (such as those described herein).

For example, in particular embodiments of the fourth to fifth aspects of the invention, the additional therapeutic agent is an agent that:
(i) is capable of reducing blood sugar levels; and/or
(ii) is an insulin sensitizer; and/or
(iii) is able to enhance insulin release,
which agents will be readily identified by those skilled in the art and include, in particular, such therapeutic agents that are commercially available (e.g. agents that the subject of a marketing authorization in one or more territory, such as a European or US marketing authorization).

The skilled person will understand that references to therapeutic agents capable of reducing blood glucose levels may refer to compounds capable of reducing levels of blood by at least 10% (such as at least 20%, at least 30% or at least 40%, for example at least 50%, at least 60%, at least 70% or at least 80%, e.g. at least 90%) when compared to the blood glucose levels prior to treatment with the relevant compound.

In alternative embodiments of the sixth and seventh aspects of the invention, the additional therapeutic agent is an agent for the treatment or prevention of a non-alcoholic fatty liver disease (such as NASH), which agents will be readily identified by those skilled in the art and include, in particular, such therapeutic agents that are commercially available (e.g. agents that the subject of a marketing authorization in one or more territory, such as a European or US marketing authorization).

In alternative embodiments of the sixth and seventh aspects of the invention, the additional therapeutic agent is an agent for treating a disease or disorder the treatment of which is mediated by activation of the β₂ adrenergic receptor, which diseases and disorders will include those described herein, and which agents will be readily identified by those skilled in the art and include, in particular, such therapeutic agents that are commercially available (e.g. agents that the subject of a marketing authorization in one or more territory, such as a European or US marketing authorization).

### Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable adjuvant, diluent or carrier.

In further aspects of the invention, there is provided a process for the preparation of a combination product or kit-of-parts as hereinbefore defined, which process comprises bringing into association a compound of the invention, as hereinbefore defined, or a pharmaceutically acceptable salt thereof with the other therapeutic agent that is useful in the treatment of hyperglycemia or a disorder characterized by hyperglycemia (e.g. type 2 diabetes), and at least one pharmaceutically-acceptable adjuvant, diluent or carrier.

As used herein, references to bringing into association will mean that the two components are rendered suitable for administration in conjunction with each other.

Thus, in relation to the process for the preparation of a kit of parts as hereinbefore defined, by bringing the two components “into association with” each other, we include that the two components of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds as defined in the first aspect of the invention (i.e. compounds of the invention) may be prepared in accordance with techniques that are well known to those skilled in the art, such as those described in the examples provided hereinafter.

For example, there is provided a process for the preparation of a compound of formula I, or a pharmaceutically acceptable salt thereof, as defined in the first aspect of the invention, which process comprises:
(i) reaction of a compound of formula II

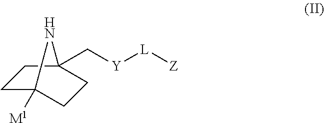

wherein Y, L and Z as defined herein above, and wherein M¹ represents a suitable metal or metal halide (e.g. Li), with a compound of formula III

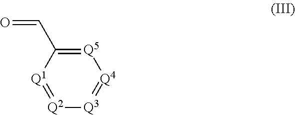

wherein the ring comprising Q¹ to Q⁵ is as defined herein above, under conditions known to those skilled in the art, such as in a suitable solvent (e.g. diethyl ether) and optionally in the presence of a suitable base (such as TMEDA);
(ii) reaction of a compound of formula IV

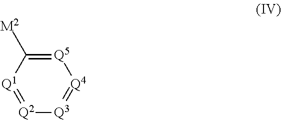

wherein the ring comprising Q¹ to Q⁵ is as defined herein, and wherein M² represents a suitable metal or metal halide (e.g. a metal bromide, such as MgBr), with a compound of formula V

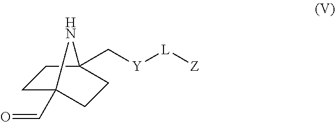

wherein Y, L and Z are as defined herein, under conditions known to those skilled in the art, such as in a suitable solvent (e.g. THF);
(iiia) reaction of a compound of formula VI

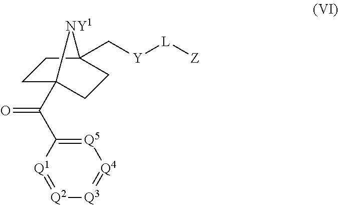

wherein Q¹ to Q⁵, Y, L and Z are as defined hereinabove and Y¹ represents H or PG¹, wherein PG¹ is a suitable protecting group as known to those skilled in the art (e.g. —C(O)OtBu or —SO₂CH₃), with a suitable reduction agent as known to those skilled in the art (such as NaBH₄ or LiAlH₄) or by hydrogenation in the presence of a suitable catalyst;
(iiib) for compounds of formula IA (or analogously for compounds of formula IB), reaction of a compound of formula VI as defined herein above in the presence of a suitable catalyst (such as a complex between (1S, 2S)-(+)-N-(4-toluenesulphonyl)-1,2-diphenylethylene diamine and [Ru (cymene)Cl₂]₂)) and in the presence of hydrogen or a suitable hydrogen donor (such as formic acid) and optionally in the presence of a base (e.g. Et₃N) and in the presence of a suitable solvent (such as CH₂Cl₂);
(iv) deprotection of a compound of formula VII

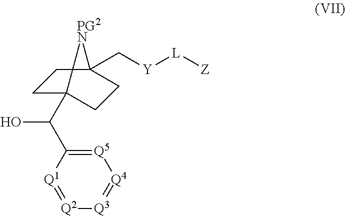

wherein the ring comprising Q¹ to Q⁵, and Y, L and Z are as defined hereinabove, and PG² represents a suitable protecting group as known to those skilled in the art (e.g. a carbamate protecting group, such as tert-butyloxycarbonyl (Boc), fluorenyl-methyloxycarbonyl (Fmoc) or carboxybenzyl (Cbz), or an amide protecting group, such as acetyl and benzoyl), under conditions known to those skilled in the art (for example in the case of Boc, in the presence of a suitable acid (e.g. trifluoroacetic acid or HCl).

Compounds of formulae II, III, IV, V, VI and VII are either commercially available, are known in the literature, or may be obtained either by analogy with the processes described herein, or by conventional synthetic procedures, in accordance with standard techniques, from available starting materials (e.g. appropriately substituted benzaldehydes, styrenes or phenacyl bromides (or phenacylchloride, and the like) using appropriate reagents and reaction conditions. In this respect, the skilled person may refer to inter alia “Comprehensive Organic Synthesis” by B. M. Trost and I. Fleming, Pergamon Press, 1991. Further references that may be employed include “Science of Synthesis”, Volumes 9-17 (Hetarenes and Related Ring Systems), Georg Thieme Verlag, 2006.

The substituents X¹, X² G¹, G² and G³, as hereinbefore defined, may be modified one or more times, after or during the processes described above for preparation of compounds of formula I by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Such compounds may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula I).

The skilled person will understand that compounds of formula I having specific stereochemistry may be provided by reacting suitable starting materials having the required stereochemistry in processes as described herein. Further, the skilled person will understand that suitable starting materials having the required stereochemistry may be prepared by analogy with the processes described herein.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

Compounds as described herein (in particular, compounds as defined in the first and, therefore, second and third aspects of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, such compounds may have the advantage that they are more efficacious and/or exhibit advantageous properties in vivo.

Without wishing to be bound by theory, compounds as described herein are thought to be potent agonists of the β2-adrenergic receptor, which allows for increased glucose uptake in skeletal muscle cells.

In addition, compounds as described herein are thought to be agonists of the β2-adrenergic receptor without (or with only a minimal effect in) inducing CAMP production. It is thought that this allows for effects such as the increased glucose uptake in skeletal muscle cells with lower levels of side effects than would result from other treatments. Further, combining compounds as described herein with other therapeutic agents, such as those that are able to decrease blood glucose levels, is thought to provide an effective combination therapy.

### EXAMPLES

The present invention is illustrated by way of the following examples.

Chemicals and reagents were obtained from commercial suppliers and were used as received unless otherwise stated. All reactions involving moisture sensitive reagents were performed in oven or flame dried glassware under a positive pressure of nitrogen or argon.

### Abbreviations

Abbreviations as used herein will be known to those skilled in the art. In particular, the following abbreviations may be used herein.
aq aqueous
DIPEA N,N-diisopropylethylamine
rt room temperature
sat saturated
TBAF tetrabutylammonium fluoride
TMEDA N,N,N′,N′-tetramethylethylenediamine
TMSOTf trimethylsilyl trifluoromethanesulfonate
XPhos dicyclohexyl[2′,4′,6′-tris(propan-2-yl) [1,1′-biphenyl]-2-yl]phosphane

### EXAMPLE COMPOUNDS

In the event that there is a discrepancy between nomenclature and the structure of compounds as depicted graphically, it is the latter that presides (unless contradicted by any experimental details that may be given and/or unless it is clear from the context).

### Example 1: (R)-(4-(((4-Chlorobenzyl)oxy)methyl)-7-azabicyclo[2.2.1]heptan-1-yl)(3-fluorophenyl)methanol

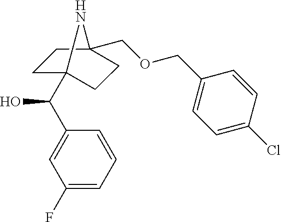 [SMILES: O[C@H](c1cccc(F)c1)C12CCC(COCc3ccc(Cl)cc3)(CC1)N2]

### (a) tert-Butyl 7-azabicyclo[2.2.1]heptane-7-carboxylate

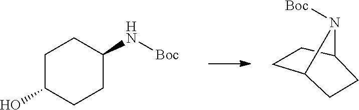 [SMILES: CC(C)(C)OC(=O)N[C@H]1CC[C@H](O)CC1 ; CC(C)(C)OC(=O)N1C2CCC1CC2]

Methanesulfonyl chloride (3.20 mL, 41.8 mmol) was added to a stirred ice-cooled mixture of (trans) tert-butyl-4-hydroxycyclohexyl) carbamate (6 g, 27.9 mmol), Et₃N (5.80 mL, 41.9 mmol) and CH₂Cl₂ (200 mL). The cooling bath was removed and the mixture was stirred at rt for 1 h. Et₃N (5.80 mL, 41.9 mmol) and methanesulfonyl chloride (3.20 mL, 41.8 mmol) were added at rt and stirring was continued for 1 h. Additional portions of Et₃N (5.80 mL, 41.9 mmol) and methanesulfonyl chloride (3.20 mL, 41.8 mmol) were added at rt and stirring was continued for 30 min. NaHCO₃(aq, sat) was carefully added and the mixture was extracted with CH₂Cl₂. The organic phase was washed with water and brine, dried (Mg₂SO₄) and concentrated. The residue was dissolved in THF (200 mL), cooled in an ice bath and tBuOK (9.38 g, 83.6 mmol) was added in portions. The cooling bath was removed and the mixture was stirred at rt overnight. Crushed ice was added and the mixture was stirred until the ice melted. The mixture was extracted with Et₂O. The aq phase was extracted with Et₂O and the combined organic phases were washed with water and brine, dried (Na₂SO₄) and concentrated. The residue was purified by chromatography to give the sub-title compound (4.16 g, 76%).

### (b) tert-Butyl 1-(((tert-butyldimethylsilyl)oxy)methyl)-7-azabicyclo[2.2.1]heptane-7-carboxylate

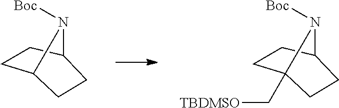 [SMILES: CC(C)(C)OC(=O)N1C2CCC1CC2 ; CC(C)(C)OC(=O)N1C2CCC1(CO[Si](C)(C)C(C)(C)C)CC2]

sec-BuLi (1.3 M in cyclohexane/hexane (92/8), 17.5 ml, 22.8 mmol) was added dropwise to a stirred solution of tert-butyl-7-azabicyclo[2.2.1]heptane-7-carboxylate (3.00 g, 15.2 mmol) and TMEDA (3.4 ml, 22.8 mmol) in Et₂O (30 mL) at −78° C. The mixture was stirred at −78° C. for 15 min and DMF (2.35 mL, 30.4 mmol was added. The mixture was stirred at −78° C. for 90 min and NH₄Cl (aq, sat, 8 mL) was added. The mixture was allowed to warm to rt and the layers were separated. The aq phase was extracted with Et₂O and the combined organic phases were washed with brine, dried (Na₂SO₄) and concentrated. The residue was dissolved in MeOH (40 mL), cooled in an ice-bath, and NaBH₄ (0.69 g, 18 mmol) was added. The mixture was stirred overnight while letting the temperature come to rt. H₂O (10 mL) was carefully added and the volatiles were evaporated. The mixture was extracted with EtOAc and the combined extracts were washed with brine, dried (Na₂SO₄) and concentrated. The residue was dissolved in DMF (10 mL) and imidazole (1.86 g 27 mmol) followed by tert-butyldimethylsilyl chloride (3.40 mg, 22.8 mmol) were added at rt. The mixture was stirred at rt for 2 h. H₂O was added and the mixture was extracted with Et₂O. The combined extracts were washed with brine, dried (MgSO₄) and concentrated and the residue was purified by chromatography to give the subtitle compound (2.70 g, 52%).

### (c) tert-Butyl 1-(((tert-butyldimethylsilyl)oxy)methyl)-4-(hydroxymethyl)-7-azabicyclo[2.2.1]heptane-7-carboxylate

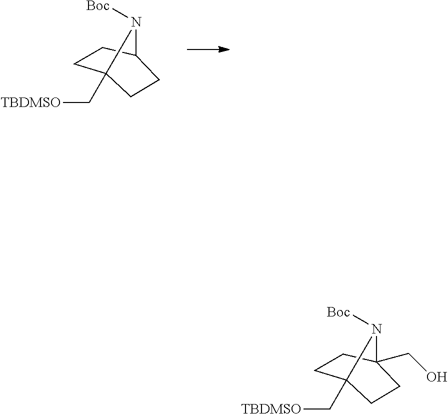 [SMILES: CC(C)(C)OC(=O)N1C2CCC1(CO[Si](C)(C)C(C)(C)C)CC2 ; CC(C)(C)OC(=O)N1C2(CO)CCC1(CO[Si](C)(C)C(C)(C)C)CC2]

sec-BuLi (1.3 M in cyclohexane, 1.40 mL, 1.86 mmol) was added dropwise to a stirred mixture of tert-butyl 1-(((tert-butyldimethylsilyl)oxy)methyl)-7-azabicyclo[2.2.1]heptane-7-carboxylate (423 mg, 1.24 mmol), TMEDA (0.28 mL, 1.86 mmol) and Et₂O (4 mL) at −78° C. The mixture was stirred at −78° C. for 1 h and DMF (141 μL, 2.48 mmol) was rapidly added. The mixture was stirred at −78° C. for 1 h. NH₄Cl (aq, sat) was added and the mixture was allowed to come to rt and extracted with Et₂O. The combined extracts were dried (Na₂SO₄) and concentrated. The residue was dissolved in MeOH (6 mL), cooled to 0° C., and NaBH₄ (56 mg, 1.24 mmol) was added in portions. After 1 h at 0° C., H₂O (2 mL) was added and the volatiles were evaporated. The mixture was extracted with EtOAc, and the combined extracts were washed with brine, dried (Na₂SO₄) and concentrated. The residue was purified by chromatography to give the sub-title compound (372 mg, 82%).

### (d) tert-Butyl 1-(((tert-butyldimethylsilyl)oxy)methyl)-4-(((4-chlorobenzyl)-oxy)methyl)-7-azabicyclo[2.2.1]heptane-7-carboxylate

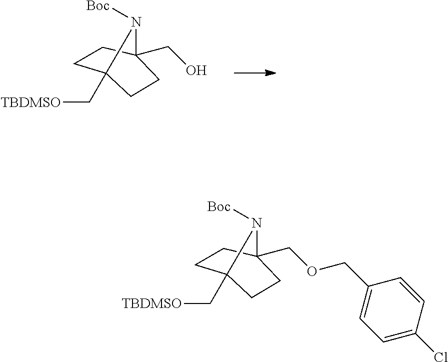 [SMILES: CC(C)(C)OC(=O)N1C2(CO)CCC1(CO[Si](C)(C)C(C)(C)C)CC2 ; CC(C)(C)OC(=O)N1C2(COCc3ccc(Cl)cc3)CCC1(CO[Si](C)(C)C(C)(C)C)CC2]

NaH (60% suspension in mineral oil, 86 mg, 2.15 mmol) was washed four times with Et₂O and suspended in THF (2 mL). The mixture was cooled in an ice-bath and a solution of tert-butyl 1-(((tert-butyldimethylsilyl)oxy)methyl)-4-(hydroxymethyl)-7-aza-bicyclo[2.2.1]heptane-7-carboxylate (200 mg, 0.54 mmol) in THF (2 mL) was added dropwise. The mixture was stirred at 0° C. for 30 min, and a solution of 1-(bromomethyl)-4-chlorobenzene (332 mg, 1.61 mmol) in THF (1.5 mL) was added. The mixture was stirred at 50° C. for 18 h and allowed to come to rt. H₂O was added and the mixture was extracted with EtOAc. The combined extracts were washed with brine, dried (Na₂SO₄) and concentrated. The residue was purified by chromatography to give the sub-title compound (235 mg, 88%).

### (e) tert-Butyl 1-(((4-chlorobenzyl)oxy)methyl)-4-(hydroxymethyl)-7-azabicyclo-[2.2.1]heptane-7-carboxylate

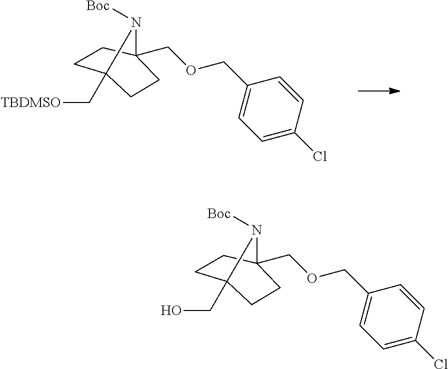 [SMILES: CC(C)(C)OC(=O)N1C2(COCc3ccc(Cl)cc3)CCC1(CO[Si](C)(C)C(C)(C)C)CC2 ; CC(C)(C)OC(=O)N1C2(CO)CCC1(COCc1ccc(Cl)cc1)CC2]

TBAF (1 M in THF, 1.33 mL, 1.33 mM) was added dropwise to an ice-cooled solution of tert-butyl 1-(((tert-butyldimethylsilyl)oxy)methyl)-4-(((4-chlorobenzyl)oxy)methyl)-7-azabicyclo[2.2.1]heptane-7-carboxylate (220 mg, 0.44 mmol) in THF (10 mL). The mixture was stirred at rt for 16 h, diluted with Et₂₀ (25 mL), washed with water, brine, dried (Na₂SO₄) and concentrated. The residue was purified by chromatography to give the sub-title compound (145 mg, 86%).

### (f) tert-Butyl 1-(((4-chlorobenzyl)oxy)methyl)-4-formyl-7-azabicyclo[2.2.1]heptane-7-carboxylate

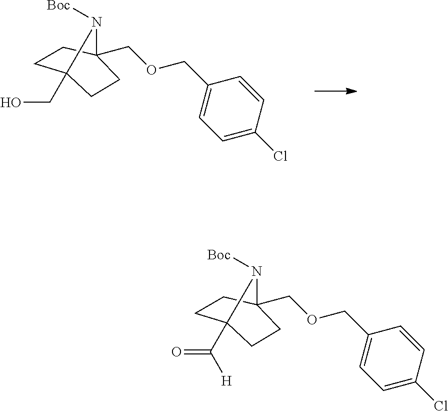 [SMILES: CC(C)(C)OC(=O)N1C2(CO)CCC1(COCc1ccc(Cl)cc1)CC2 ; CC(C)(C)OC(=O)N1C2(C=O)CCC1(COCc1ccc(Cl)cc1)CC2]

A solution of DMSO (59 μL, 0.83 mmol) in CH₂Cl₂ (0.6 mL) was added to a stirred solution of oxalyl chloride (41 μL, 0.47 mmol) in CH₂Cl₂ (0.6 mL) at −78° C. and stirred at −78° C. for 30 min. A solution of tert-butyl 1-benzyl-4-(hydroxymethyl)-7-azabicyclo[2.2.1]heptane-7-carboxylate (127 mg, 0.33 mmol) in CH₂Cl₂ (0.6 mL) was added dropwise and the mixture was stirred at −78° C. for 30 min. Et₃N (232 μL, 1.66 mmol) was added dropwise and the mixture was stirred at −78° C. for 5 min, at 0° C. for 1 h and at rt for 30 min. Water was added and the mixture was stirred for 10 min. The layers were separated and the aq phase extracted with CH₂Cl₂. The combined organic phases were washed with brine, dried (MgSO₄) and concentrated. The residue was dissolved in Et₂O, filtered through Celite and concentrated to give the sub-title compound (117 mg, 93%).

### (g) tert-Butyl 1-(((4-chlorobenzyl)oxy)methyl)-4-((R)-(3-fluorophenyl)-(hydroxy)methyl)-7-azabicyclo[2.2.1]heptane-7-carboxylate

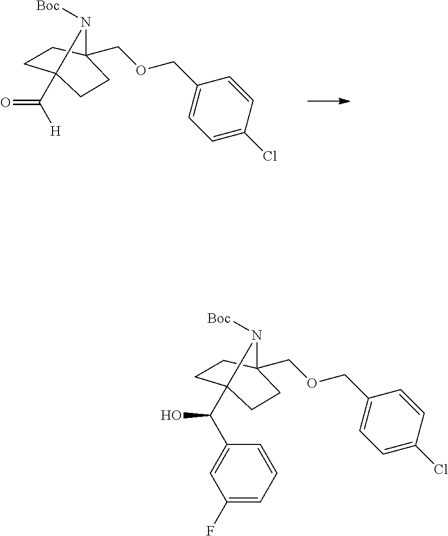 [SMILES: CC(C)(C)OC(=O)N1C2(C=O)CCC1(COCc1ccc(Cl)cc1)CC2 ; CC(C)(C)OC(=O)N1C2(COCc3ccc(Cl)cc3)CCC1([C@H](O)c1cccc(F)c1)CC2]

3-Fluorophenyl magnesium bromide (0.9 M in THF, 939 μL, 0.85 mmol) was added dropwise to a stirred solution of tert-butyl 1-(((4-chlorobenzyl)oxy)methyl)-4-formyl-7-azabicyclo[2.2.1]heptane-7-carboxylate (107 mg, 0.28 mmol) in THF (2 mL) at −78° C. The mixture was stirred at −78° C. for 90 min and quenched with NH₄Cl (aq, sat). The mixture was extracted with Et₂₀ and the combined extract were washed with brine, dried (MgSO₄) and concentrated. The residue was purified by chromatography on silica-gel followed by chiral chromatography (DAICEL CHIRALPAK ID 250×30 mm-5 μm, 40 mL/min, λ254 nm, eluent system 10% then 20% iPrOH in heptane). Concentration of the relevant fractions gave the title compound (46 mg, 34%, ee >99%, [α]_{D}²⁰=−35.7 (c=0.39, CHCl₃)), and the corresponding(S)-enantiomer (48 mg, 36%, ee >99%, [α]_{D}²⁰=+40.0 (c=0.40, CHCl₃)).

### (h) (R)-(4-(((4-Chlorobenzyl)oxy)methyl)-7-azabicyclo[2.2.1]heptan-1-yl)-(3-fluorophenyl)methanol

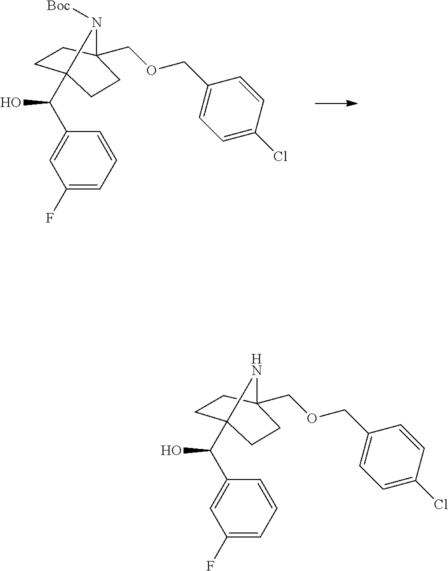 [SMILES: CC(C)(C)OC(=O)N1C2(COCc3ccc(Cl)cc3)CCC1([C@H](O)c1cccc(F)c1)CC2 ; O[C@H](c1cccc(F)c1)C12CCC(COCc3ccc(Cl)cc3)(CC1)N2]

2,6-Lutidine (195 μL, 1.68 mmol) and TMSOTf (76 μL, 0.42 mmol) were added to a solution of tert-butyl 1-(((4-chlorobenzyl)oxy)methyl)-4-((R)-(3-fluorophenyl)(hydroxy)methyl)-7-azabicyclo[2.2.1]heptane-7-carboxylate (40 mg, 84 μmol) in CH₂Cl₂ (2 mL) at rt. The mixture was stirred at rt for 16 h. NaOH (aq, 1 M, 2 mL) was added and the layers were separated. The aq phase was extracted with CH₂Cl₂ and the combined organic phases were dried (Na₂SO₄) and concentrated. The residue was dissolved in MeOH (4 mL) and NH₄F (47 mg, 1.26 mmol) was added. The mixture was stirred at rt for 1 h and concentrated. The residue was dissolved in H2O (1 mL) and NaOH (aq, 1 M, 1.5 mL) was added, and the mixture was extracted with EtOAc. The combined extracts were washed with brine, dried (Na₂SO₄) and concentrated. The residue was dissolved in CH₂Cl₂, filtered through a plug of amino-functionalized silica-gel, and concentrated to give the title compound (21 mg, 66%).

[α]_{D}²⁰=−10.7 (c=0.56, CHCl₃)

¹H NMR (400 MHZ, CDCl₃): δ 7.34-7.21 (m, 5H), 7.15-7.09 (m, 2H), 7.00-6.91 (m, 1H), 4.92 (s, 1H), 4.51 (s, 2H), 3.60 (s, 2H), 1.99-1.87 (m, 1H), 1.79-1.59 (m, 3H), 1.52-1.30 (m, 3H), 1.18 (ddd, J=11.8, 9.2, 4.7 Hz, 1H).

### Example 2: (S)-(4-(((4-Chlorobenzyl)oxy)methyl)-7-azabicyclo[2.2.1]heptan-1-yl)(3-fluorophenyl)methanol

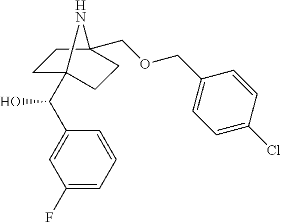 [SMILES: O[C@@H](c1cccc(F)c1)C12CCC(COCc3ccc(Cl)cc3)(CC1)N2]

The title compound was prepared from tert-butyl 1-(((4-chlorobenzyl)oxy)methyl)-4-((S)-(3-fluorophenyl)(hydroxy)methyl)-7-azabicyclo[2.2.1]heptane-7-carboxylate, see Example 1, Step (g) in accordance with the procedures in Example 1, Step (h).

[α]_{D}²⁰=+12.40 (c=0.52, CHCl₃).

The ¹H NMR spectrum was identical to the one for the (R) enantiomer in Example 1.

### Example 3: (R)-(4-(((4-Fluorobenzyl)oxy)methyl)-7-azabicyclo[2.2.1]heptan-1-yl)(3-fluorophenyl)methanol

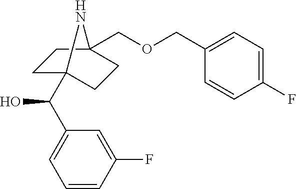 [SMILES: O[C@H](c1cccc(F)c1)C12CCC(COCc3ccc(F)cc3)(CC1)N2]

The title compound was prepared in accordance with the procedures in Example 1, using 1-(bromomethyl)-4-fluorobenzene in step (d).

[α]_{D}²⁰=−11 (c=0.75, CHCl₃)

¹H NMR (400 MHZ, CDCl₃): δ 7.33-7.24 (3H, m) 7.15-7.09 (2H, m) 7.06-7.00 (2H, m) 6.98-6.92 (1H, m) 4.90 (1H, s) 4.52 (2H, s) 3.62-3.60 (2H, s) 1.96-1.86 (1H, m) 1.80-1.71 (1H, m) 1.69-1.59 (2H, m) 1.53-1.33 (3H, m) 1.23-1.13 (1H, m).

### Example 4: (S)-(4-(((4-Fluorobenzyl)oxy)methyl)-7-azabicyclo[2.2.1]heptan-1-yl)(3-fluorophenyl)methanol

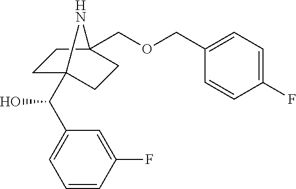 [SMILES: O[C@@H](c1cccc(F)c1)C12CCC(COCc3ccc(F)cc3)(CC1)N2]

The title compound was prepared in accordance with the procedures in Example 1, using 1-(bromomethyl)-4-fluorobenzene in step (d).

[α]_{D}²⁰=+11 (c=0.69, CHCl₃)

The ¹H NMR spectrum was identical to the one for the (R) enantiomer in Example 3.

### Example 5: (R)-(4-(((4-Fluorobenzyl)oxy)methyl)-7-azabicyclo[2.2.1]heptan-1-yl)(5-fluoropyridin-3-yl)methanol

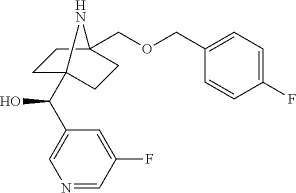 [SMILES: O[C@H](c1cncc(F)c1)C12CCC(COCc3ccc(F)cc3)(CC1)N2]

The title compound was prepared in accordance with the procedures in Example 1, using 1-(bromomethyl)-4-fluorobenzene in step (d) and 3-(5-fluoropyridin-3-yl) magnesium bromide in Step (g).

[α]_{D}²⁰=−18 (c=0.66, CHCl₃)

¹H NMR (400 MHZ, CDCl₃): δ 8.40-8.36 (2H, m) 7.53-7.48 (1H, m) 7.32-7.26 (2H, m) 7.06-6.99 (2H, m) 4.99 (1H, s) 4.52 (2H, s) 3.61 (2H, s) 1.96-1.86 (1H, m) 1.85-1.75 (1H, m) 1.70-1.60 (2H, m) 1.56-1.48 (1H, m) 1.46-1.34 (2H, m) 1.16-1.07 (1H, m).

### Example 6: (S)-(4-(((4-Fluorobenzyl)oxy)methyl)-7-azabicyclo[2.2.1]heptan-1-yl)(5-fluoropyridin-3-yl)methanol

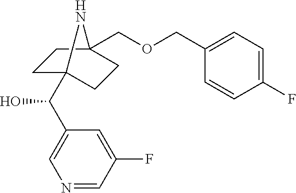 [SMILES: O[C@@H](c1cncc(F)c1)C12CCC(COCc3ccc(F)cc3)(CC1)N2]

The title compound was prepared in accordance with the procedures in Example 1, using 1-(bromomethyl)-4-fluorobenzene in step (d) and 3-(5-fluoropyridin-3-yl) magnesium bromide in Step (g).

[α]_{D}²⁰=+21 (c=0.75, CHCl₃)

The ¹H NMR spectrum was identical to the one for the (R) enantiomer in Example 5.

### Example 7: (R)-(4-Benzyl-7-azabicyclo[2.2.1]heptan-1-yl)(3-fluorophenyl)methanol

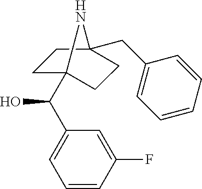 [SMILES: O[C@H](c1cccc(F)c1)C12CCC(Cc3ccccc3)(CC1)N2]

### (a) tert-Butyl 1-benzyl-4-(((tert-butyldimethylsilyl)oxy)methyl)-7-azabicyclo-[2.2.1]heptane-7-carboxylate

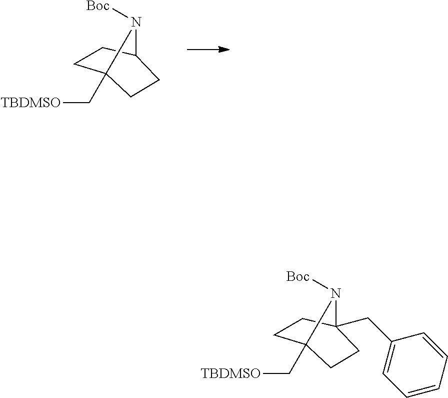 [SMILES: CC(C)(C)OC(=O)N1C2CCC1(CO[Si](C)(C)C(C)(C)C)CC2 ; CC(C)(C)OC(=O)N1C2(CO[Si](C)(C)C(C)(C)C)CCC1(Cc1ccccc1)CC2]

sec-BuLi (1.3 M in cyclohexane, 0.53 mL, 0.69 mmol) was added dropwise to a solution of tert-butyl 1-(((tert-butyldimethylsilyl)oxy)methyl)-7-azabicyclo[2.2.1]heptane-7-carboxylate (215 mg, 0.63 mmol) and TMEDA (104 μL, 0.69 mmol) in Et₂O (3.0 mL) at −78° C. The mixture was stirred at −78° C. for 2 h and a solution of CuCN·2LiCl (0.3 M in anhydrous degassed THF, 2.1 mL, 0.63 mmol) was added dropwise and stirring was continued at −78° C. for 60 min. Benzyl bromide (82 μL, 0.69 mmol) was added dropwise and the mixture was allowed to warm to rt and stirred overnight. NH₄Cl (aq, sat) was added and the mixture was extracted with CH₂Cl₂ and the combined organic phases were washed with brine, dried (Na₂SO₄) and concentrated. The residue was purified by chromatography to give the sub-title compound (189 mg, 70%).

### (b) tert-Butyl 1-benzyl-4-(hydroxymethyl)-7-azabicyclo[2.2.1]heptane-7-carboxylate

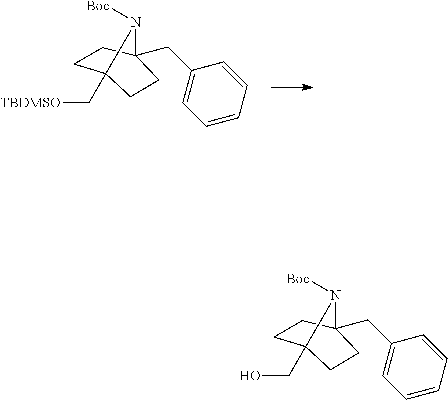 [SMILES: CC(C)(C)OC(=O)N1C2(CO[Si](C)(C)C(C)(C)C)CCC1(Cc1ccccc1)CC2 ; CC(C)(C)OC(=O)N1C2(CO)CCC1(Cc1ccccc1)CC2]

TBAF (1 M in THF, 1.6 mL, 1.6 mmol) was added dropwise to a solution of tert-butyl 1-benzyl-4-(((tert-butyldimethylsilyl)oxy)methyl)-7-azabicyclo[2.2.1]heptane-7-carboxylate (227 mg, 0.53 mmol) in THF (10 mL) at 0° C. The mixture was allowed to warm to rt and was stirred at rt for 16 h. Et₂O was added and the mixture was washed with brine, dried (Na₂SO₄) and concentrated. The residue was purified by chromatography to give the sub-title compound (152 mg, 91%).

### (c) tert-Butyl 1-benzyl-4-formyl-7-azabicyclo[2.2.1]heptane-7-carboxylate

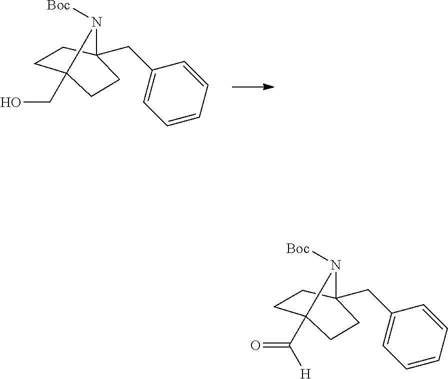 [SMILES: CC(C)(C)OC(=O)N1C2(CO)CCC1(Cc1ccccc1)CC2 ; CC(C)(C)OC(=O)N1C2(C=O)CCC1(Cc1ccccc1)CC2]

A solution of DMSO (85 μL, 1.20 mmol) in CH₂Cl₂ (0.7 mL) was added to a stirred solution of oxalyl chloride (59 μL, 0.67 mmol) in CH₂Cl₂ (0.7 mL) at −78° C. and stirred at −78° C. for 30 min. A solution of tert-butyl 1-benzyl-4-(hydroxymethyl)-7-azabicyclo[2.2.1]heptane-7-carboxylate (152 mg, 0.48 mmol) in CH₂Cl₂ (0.7 mL) was added dropwise and the mixture was stirred at −78° C. for 30 min. Et₃N (334 μL, 2.40 mmol) was added dropwise and the mixture was stirred at −78° C. for 5 min, at 0° C. for 1 h and at rt for 30 min. Water was added and the mixture was stirred for 10 min. The layers were separated and the aq phase extracted with CH₂Cl₂. The combined organic phases were washed with brine, dried (MgSO₄) and concentrated. The residue was dissolved in Et₂O, filtered through Celite and concentrated to give the sub-title compound (148 mg, 98%).

### (d) tert-Butyl 1-benzyl-4-((R)-(3-fluorophenyl)(hydroxy)methyl)-7-azabicyclo-[2.2.1]heptane-7-carboxylate

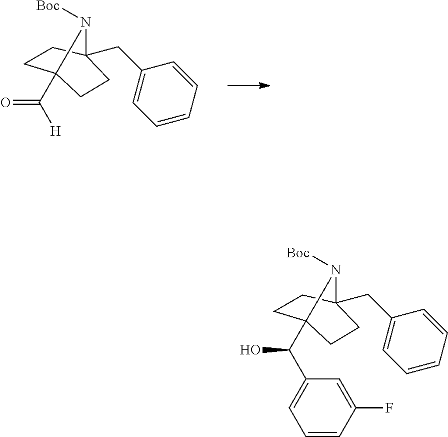 [SMILES: CC(C)(C)OC(=O)N1C2(C=O)CCC1(Cc1ccccc1)CC2 ; CC(C)(C)OC(=O)N1C2(Cc3ccccc3)CCC1([C@H](O)c1cccc(F)c1)CC2]

3-Fluorophenyl magnesium bromide (0.9 M in THF, 1.1 mL, 1.01 mmol) was added dropwise to a stirred solution of tert-butyl 1-benzyl-4-formyl-7-azabicyclo[2.2.1]heptane-7-carboxylate (159 mg, 0.50 mmol) in THF (4 mL) at −78° C. The mixture was stirred at −78° C. for 90 min and quenched with NH₄Cl (aq, sat). The mixture was extracted with Et₂O and the combined extract were washed with brine, dried (MgSO₄) and concentrated. The residue was purified by chromatography on silica-gel followed by chiral chromatography (DAICEL CHIRALPAK IG 250×30 mm-5 μm, 40 mL/min, λ 260 nm, eluent system 20% iPrOH in heptane). Concentration of the relevant eluents gave the title compound (68 mg, 41%, ee >99%, [α]_{D}²⁰=−48.59 (c=1.06, CHCl₃)), and the corresponding(S)-enantiomer (63 mg, 38%, ee >99%, [α]_{D}²⁰=+52.43 (c=1.00, CHCl₃)).

### (e) (R)-(4-Benzyl-7-azabicyclo[2.2.1]heptan-1-yl)(3-fluorophenyl)methanol

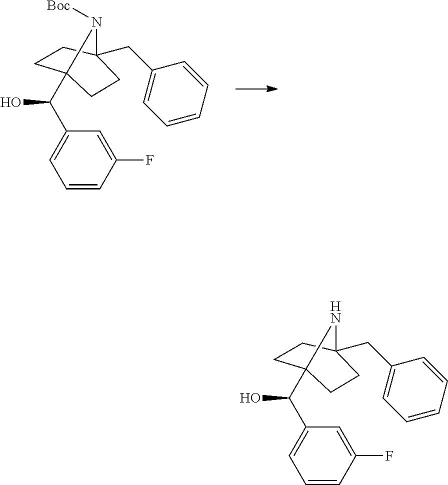 [SMILES: CC(C)(C)OC(=O)N1C2(Cc3ccccc3)CCC1([C@H](O)c1cccc(F)c1)CC2 ; O[C@H](c1cccc(F)c1)C12CCC(Cc3ccccc3)(CC1)N2]

2,6-Lutidine (328 μL, 2.82 mmol) and TMSOTf (128 μL, 0.70 mmol) were added to a solution of tert-butyl 1-benzyl-4-((R)-(3-fluorophenyl)(hydroxy)methyl)-7-azabicyclo-[2.2.1]heptane-7-carboxylate (58 mg, 0.14 mmol) in CH₂Cl₂ (3 mL) at rt. The mixture was stirred at rt for 16 h. NaOH (aq, 1 M, 2 mL) was added and the layers were separated. The aq phase was extracted with CH₂Cl₂ and the combined organic phases were dried (Na₂SO₄) and concentrated. The residue was dissolved in MeOH (6 mL) and NH₄F (78 mg, 2.1 mmol) was added. The mixture was stirred at rt for 1 h and concentrated. The residue was dissolved in H₂O (1 mL) and NaOH (aq, 1 M, 2 mL) was added. The mixture was extracted with EtOAc and the combined extracts were washed with brine, dried (Na₂SO₄) and concentrated. The residue was purified by chromatography to give the title compound (32 mg, 72%).

[α]_{D}²⁰−10.41 (c=0.96, CHCl₃)

¹H NMR (400 MHZ, CDCl₃) δ 7.35-7.15 (m, 6H), 7.14-7.03 (m, 2H), 6.98-6.87 (m, 1H), 4.94 (s, 1H), 3.50-2.50 (br s, 2H), 2.95, 2.94 (AB q, J=13.64 Hz, 2H) (overlapping), 2.00-1.80 (m, 1H), 1.69-1.37 (m, 5H), 1.34-1.18 (m, 1H), 1.15-1.03 (m, 1H).

### Example 8: (S)-(4-Benzyl-7-azabicyclo[2.2.1]heptan-1-yl)(3-fluorophenyl)methanol

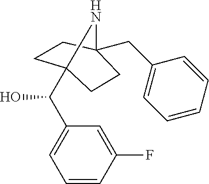 [SMILES: O[C@@H](c1cccc(F)c1)C12CCC(Cc3ccccc3)(CC1)N2]

The title compound was prepared from tert-butyl 1-benzyl-4-((S)-(3-fluorophenyl)-(hydroxy)methyl)-7-azabicyclo[2.2.1]heptane-7-carboxylate see Example 7, Step (d), in accordance with the procedure in Example 7, Step (e).

[α]_{D}²⁰=+9.2 (c=0.97, CHCl₃)

¹H NMR is identical to the one of the (R)-enantiomer in Example 7.

### Example 9: (R)-(3-Fluorophenyl)(4-(4-methoxybenzyl)-7-azabicyclo[2.2.1]heptan-1-yl)methanol

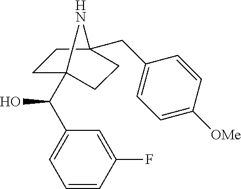 [SMILES: COc1ccc(CC23CCC([C@H](O)c4cccc(F)c4)(CC2)N3)cc1]

The title compound was prepared in accordance with the procedures in Example 7, using 4-methoxybenzyl bromide in Step (a).

[α]_{D}²⁰=−11.6 (c=0.35, MeOH)

¹H NMR (400 MHZ, CDCl₃) δ 7.30-7.22 (m, 1H), 7.15-7.07 (m, 4H), 6.98-6.90 (m, 1H), 6.86-6.80 (m, 2H), 4.90 (s, 1H), 3.80 (s, 3H), 3.5-2.8 (br s, 1H), 2.91 (s, 2H), 1.95-1.81 (m, 1H), 1.77-1.36 (m, 6H), 1.38-1.25 (m, 1H), 1.11 (ddd, J=12.0, 9.1, 4.8 Hz, 1H).

### Example 10: (S)-(3-Fluorophenyl)(4-(4-methoxybenzyl)-7-azabicyclo[2.2.1]heptan-1-yl)methanol

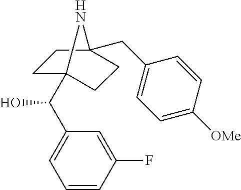 [SMILES: COc1ccc(CC23CCC([C@@H](O)c4cccc(F)c4)(CC2)N3)cc1]

The title compound was prepared in accordance with the procedures in Example 7, using 4-methoxybenzyl bromide in Step (a).

[α]_{D}²⁰=+12.2 (c=0.49, MeOH)

¹H NMR is identical to the one of the (R)-enantiomer in Example 9.

### Example 11: (R)-(4-(4-Fluorobenzyl)-7-azabicyclo[2.2.1]heptan-1-yl)-(3-fluoro-phenyl)methanol

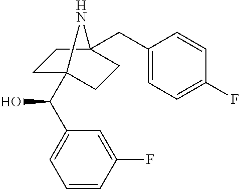 [SMILES: O[C@H](c1cccc(F)c1)C12CCC(Cc3ccc(F)cc3)(CC1)N2]

The title compound was prepared in accordance with the procedures in Example 7, using 4-fluorobenzyl bromide in Step (a).

[α]_{D}²⁰=−11.6 (c=0.35, MeOH)

¹H NMR (400 MHZ, CDCl₃) δ 7.31-7.23 (m, 1H), 7.20-7.13 (m, 2H), 7.13-7.07 (m, 2H), 7.00-6.90 (m, 3H), 4.91 (s, 1H), 2.94 (s, 2H), 2.4-1.4 (br s, 2H, overlapping), 1.94-1.81 (m, 1H), 1.66-1.39 (m, 5H), 1.35-1.22 (m, 1H), 1.13 (ddd, J=11.9, 9.0, 4.8 Hz, 1H).

### Example 12: (S)-(4-(4-Fluorobenzyl)-7-azabicyclo[2.2.1]heptan-1-yl)-(3-fluorophenyl)methanol

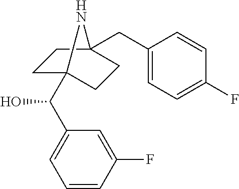 [SMILES: O[C@@H](c1cccc(F)c1)C12CCC(Cc3ccc(F)cc3)(CC1)N2]

The title compound was prepared in accordance with the procedures in Example 7, using 4-fluorobenzyl bromide in Step (a).

[α]_{D}²⁰=+14.0 (c=0.52, CHCl₃)

The ¹H NMR spectrum was identical to the one for the (R)-enantiomer in Example 11.

### Example 13: (R)-(3-Fluorophenyl)(4-(2-(trans-4-methoxycyclohexyl)ethyl)-7-azabicyclo[2.2.1]heptan-1-yl)methanol

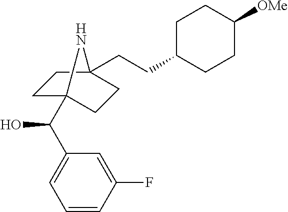 [SMILES: CO[C@H]1CC[C@H](CCC23CCC([C@H](O)c4cccc(F)c4)(CC2)N3)CC1]

### (a) trans-1-(Iodomethyl)-4-methoxycyclohexane

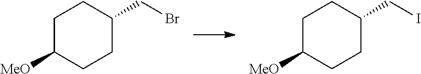 [SMILES: CO[C@H]1CC[C@H](CBr)CC1 ; CO[C@H]1CC[C@H](CI)CC1]

A mixture of trans-1-(bromomethyl)-4-methoxycyclohexane (910 mg, 4.4 mmol), NaI (2.6 g, 17.6 mmol) and acetone (6 mL) was heated in a sealed tube at 80° C. for 3 h. The mixture was cooled to rt, filtered through Celite and concentrated. The residue was treated with Et₂O, which was washed with H₂O, dried (Na₂SO₄) and concentrated to give the sub-title compound (1.12 g, 100%), which was used in the next step without further purification.

### (b) ((trans-4-Methoxycyclohexyl)methyl)triphenylphosphonium iodide

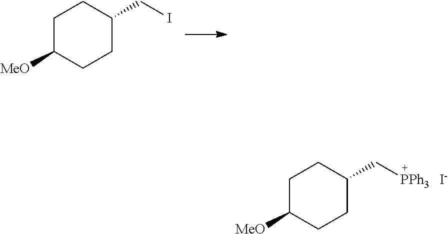

A mixture of trans-1-(iodomethyl)-4-methoxycyclohexane (1.11 g, 4.4 mmol), triphenyl phosphine (1.26 g, 4.8 mmol) and MeCN (6 mL) was heated in a sealed tube at 100° C. for 16 h, cooled to rt and concentrated. The residue was treated with toluene and sonicated until the product solidified. The mixture was filtered and the solids washed with toluene and dried to give the sub-title compound (2.25 g, 100%).

### (c) tert-Butyl 1-(((tert-butyldimethylsilyl)oxy)methyl)-4-formyl-7-azabicyclo-[2.2.1]heptane-7-carboxylate

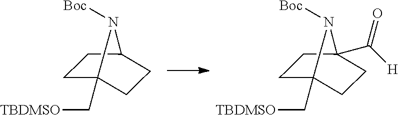 [SMILES: CC(C)(C)OC(=O)N1C2CCC1(CO[Si](C)(C)C(C)(C)C)CC2 ; CC(C)(C)OC(=O)N1C2(C=O)CCC1(CO[Si](C)(C)C(C)(C)C)CC2]

sec-BuLi (1.3 M in cyclohexane/hexane, 726 μL, 0.94 mmol) was added dropwise to a mixture of tert-butyl 1-(((tert-butyldimethylsilyl)oxy)methyl)-7-azabicyclo[2.2.1]heptane-7-carboxylate (215 mg, 0.63 mmol), TMEDA (142 μL, 0.94 mmol) and Et₂₀ (3 mL) at −78° C. The mixture was stirred at −78° C. for 1 h and DMF (97 μL, 1.26 mmol) was added. The mixture was stirred at −78° C. for 1 h. The reaction was quenched with NH₄Cl (aq, sat, 2 mL) and the mixture was extracted with Et₂O. The combined extracts were washed with brine, dried (Na₂SO₄) and concentrated. The residue was purified by chromatography to give the sub-title compound (188 mg, 80%).

### (d) tert-Butyl 1-(((tert-butyldimethylsilyl)oxy)methyl)-4-((E)-2-(trans-4-methoxycyclohexyl) vinyl)-7-azabicyclo[2.2.1]heptane-7-carboxylate

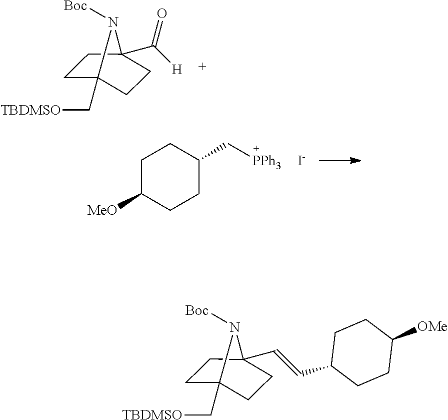

n-BuLi (2.5 M in hexane, 433 μL, 1.08 mmol) was added dropwise to a mixture of (trans-4-methoxycyclohexyl)methyl)triphenylphosphonium iodide (559 mg, 1.08 mmol) and THF (5 mL) at −78° C. The mixture was stirred at −40° C. for 40 min and cooled to −78° C. A solution of tert-butyl 1-(((tert-butyldimethylsilyl)oxy)methyl)-4-formyl-7-azabicyclo[2.2.1]heptane-7-carboxylate (200 mg, 0.54 mmol) in THF (4 mL) was added dropwise at −78° C. The mixture was stirred at −78° C. for 1 h, allowed to reach −15° C. over 1 h, quenched with NH₄Cl (aq, sat, 7 mL) and extracted with Et₂O. The combined extracts were dried (Na₂SO₄) and concentrated. The residue was purified by chromatography to give the sub-title compound (172 mg, 66%).

### (e) tert-Butyl 1-(((tert-butyldimethylsilyl)oxy)methyl)-4-(2-(trans-4-methoxy-cyclohexyl)ethyl)-7-azabicyclo[2.2.1]heptane-7-carboxylate

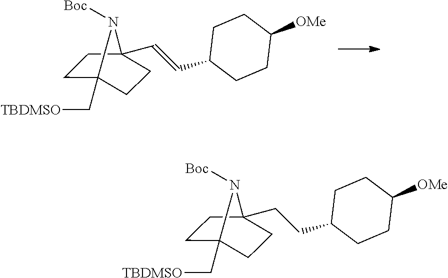 [SMILES: CO[C@H]1CC[C@H](/C=C/C23CCC(CO[Si](C)(C)C(C)(C)C)(CC2)N3C(=O)OC(C)(C)C)CC1 ; CO[C@H]1CC[C@H](CCC23CCC(CO[Si](C)(C)C(C)(C)C)(CC2)N3C(=O)OC(C)(C)C)CC1]

A mixture of tert-butyl 1-(((tert-butyldimethylsilyl)oxy)methyl)-4-((E)-2-(trans-4-methoxycyclohexyl) vinyl)-7-azabicyclo[2.2.1]heptane-7-carboxylate (250 mg, 0.52 mmol), TMEDA (0.94 mL, 6.25 mmol), p-toluenesulfonylhydrazide (776 mg, 4.2 mmol) and toluene (15 mL) was heated in a sealed tube at 110° C. for 60 h, cooled to rt and diluted with Et₂O. The mixture was washed with H₂O and brine, dried (Na₂SO₄) and concentrated. The residue was purified by chromatography to give the sub-title compound (207 mg, 83%).

### (f) tert-Butyl 1-(hydroxymethyl)-4-(2-(trans-4-methoxycyclohexyl)ethyl)-7-azabicyclo[2.2.1]heptane-7-carboxylate

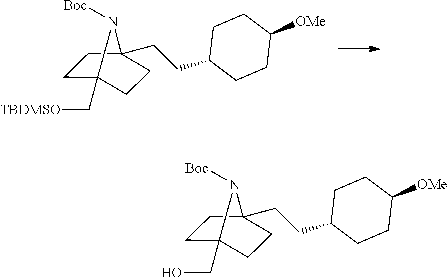 [SMILES: CO[C@H]1CC[C@H](CCC23CCC(CO[Si](C)(C)C(C)(C)C)(CC2)N3C(=O)OC(C)(C)C)CC1 ; CO[C@H]1CC[C@H](CCC23CCC(CO)(CC2)N3C(=O)OC(C)(C)C)CC1]

TBAF (1 M in THF, 1.25 mL, 1.25 mmol) was added dropwise to a solution of tert-butyl 1-(((tert-butyldimethylsilyl)oxy)methyl)-4-(2-(trans-4-methoxycyclohexyl)ethyl)-7-azabicyclo[2.2.1]heptane-7-carboxylate (200 mg, 0.42 mmol) in THF (10 mL) at 0° C. The ice-bath was removed and the mixture was stirred at rt for 16 h and diluted with Et₂O. The mixture was washed with H₂O and brine, dried (Na₂SO₄) and concentrated. The residue was purified by chromatography to give the sub-title compound (132 mg, 87%).

### (g) tert-Butyl 1-formyl-4-(2-(trans-4-methoxycyclohexyl)ethyl)-7-azabicyclo-[2.2.1]heptane-7-carboxylate

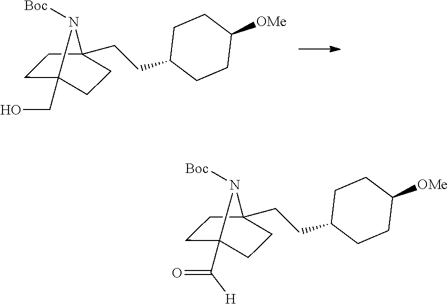 [SMILES: CO[C@H]1CC[C@H](CCC23CCC(CO)(CC2)N3C(=O)OC(C)(C)C)CC1 ; CO[C@H]1CC[C@H](CCC23CCC(C=O)(CC2)N3C(=O)OC(C)(C)C)CC1]

A solution of DMSO (58 μL, 0.82 mmol) in CH₂Cl₂ (0.75 mL) was added dropwise to a stirred mixture of oxalyl chloride (2 M in CH₂Cl₂, 229 μL, 0.46 mmol) and CH₂Cl₂ (0.75 mL) at −78° C. and stirred at −78° C. for 30 min. A solution of tert-butyl 1-(hydroxymethyl)-4-(2-(trans-4-methoxycyclohexyl)ethyl)-7-azabicyclo[2.2.1]heptane-7-carboxylate (120 mg, 0.33 mmol) in CH₂Cl₂ (1.5 mL) was added dropwise and the mixture was stirred at −78° C. for 30 min. Et₃N (228 μL, 1.63 mmol) was added dropwise and the mixture was stirred at −78° C. for 5 min, at 0° C. for 1 h and at rt for 30 min. Water was added and the mixture was stirred for 10 min. The layers were separated and the aq phase extracted with CH₂Cl₂. The combined organic phases were washed with brine, dried (MgSO₄) and concentrated. The residue was dissolved in Et₂O, filtered through Celite and concentrated to give the sub-title compound (119 mg, 100%).

### (h) tert-Butyl 1-((R)-(3-fluorophenyl)(hydroxy)methyl)-4-(2-(trans-4-methoxy-cyclohexyl)ethyl)-7-azabicyclo[2.2.1]heptane-7-carboxylate

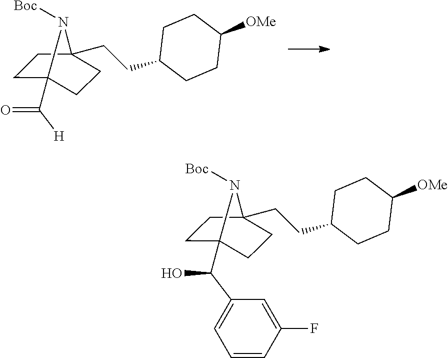 [SMILES: CO[C@H]1CC[C@H](CCC23CCC(C=O)(CC2)N3C(=O)OC(C)(C)C)CC1 ; CO[C@H]1CC[C@H](CCC23CCC([C@H](O)c4cccc(F)c4)(CC2)N3C(=O)OC(C)(C)C)CC1]

3-Fluorophenyl magnesium bromide (0.9 M in THF, 0.7 mL, 0.63 mmol) was added dropwise to a stirred solution of tert-butyl 1-formyl-4-(2-(trans-4-methoxy-cyclohexyl)ethyl)-7-azabicyclo[2.2.1]heptane-7-carboxylate (115 mg, 0.32 mmol) in THF (2 mL) at −78° C. The mixture was stirred at −78° C. for 90 min and quenched with NH₄Cl (aq, sat). The mixture was extracted with CH₂Cl₂ and the combined extract were washed with brine, dried (MgSO₄) and concentrated. The residue was purified by chromatography on silica-gel followed by chiral chromatography (DAICEL CHIRALPAK IG 250×30 mm-5 μm, 40 mL/min, λ 261 nm, eluent system 20% then 40% iPrOH in heptane). Concentration of the relevant eluents gave the title compound (62 mg, 43%), ee >99%, [α]_{D}²⁰=−42.2 (c=0.56, CHCl₃), and the corresponding(S)-enantiomer (62 mg, 43%, ee >99%, [α]_{D}²⁰=+34.2 (c=0.38, CHCl₃).

### (i) (R)-(3-Fluorophenyl)(4-(2-(trans-4-methoxycyclohexyl)ethyl)-7-azabicyclo-[2.2.1]heptan-1-yl)methanol

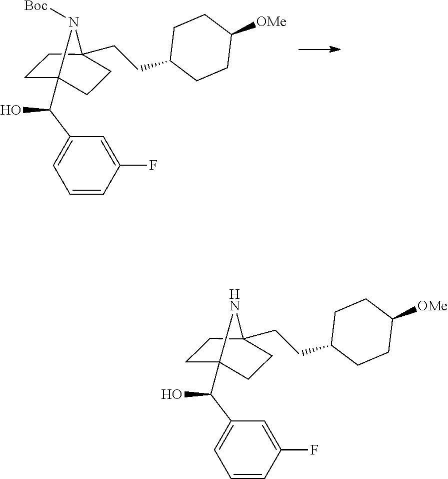 [SMILES: CO[C@H]1CC[C@H](CCC23CCC([C@H](O)c4cccc(F)c4)(CC2)N3C(=O)OC(C)(C)C)CC1 ; CO[C@H]1CC[C@H](CCC23CCC([C@H](O)c4cccc(F)c4)(CC2)N3)CC1]

2,6-Lutidine (287 μL, 2.5 mmol) and TMSOTf (112 μL, 0.62 mmol) were added to a solution of tert-butyl 1-((R)-(3-fluorophenyl)(hydroxy)methyl)-4-(2-(trans-4-methoxycyclohexyl)ethyl)-7-azabicyclo[2.2.1]heptane-7-carboxylate (57 mg, 0.12 mmol) in CH₂Cl₂ (2 mL) at rt. The mixture was stirred at rt for 16 h. NaOH (aq, 1 M, 2 mL) was added and the layers were separated. The aq phase was extracted with CH₂Cl₂ and the combined organic phases were dried (Na₂SO₄) and concentrated. The residue was dissolved in MeOH (3 mL) and NH₄F (69 mg, 1.85 mmol) was added. The mixture was stirred at rt for 1 h and concentrated. The residue was partitioned between NaOH (aq, 1 M) and CH₂Cl₂ and the layers were separated. The aq phase was extracted with CH₂Cl₂ and the combined organic phases were washed with brine, dried (Na₂SO₄) and concentrated. The residue was triturated three times with Et₂O and dried to give the title compound (39 mg, 91%).

[α]_{D}²⁰=−12.1 (c=0.33, CHCl₃)

¹H NMR (400 MHZ, CDCl₃) δ 7.32-7.23 (m, 1H), 7.17-7.07 (m, 2H), 6.98-6.91 (m, 1H), 4.93 (s, 1H), 3.34 (s, 3H), 3.13-3.00 (m, 1H), 2.10-2.01 (m, 2H), 1.96-1.84 (m, 1H), 1.84-1.75 (m, 2H), 1.71-1.27 (m, 8H), 1.26-1.09 (m, 6H), 0.98-0.85 (m, 2H).

### Example 14: (S)-(3-Fluorophenyl)(4-(2-((trans)-4-methoxycyclohexyl)ethyl)-7-aza-bicyclo[2.2.1]heptan-1-yl)methanol

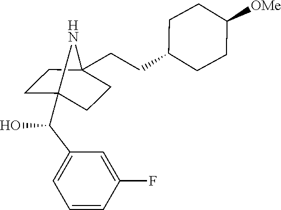 [SMILES: CO[C@H]1CC[C@H](CCC23CCC([C@@H](O)c4cccc(F)c4)(CC2)N3)CC1]

The title compound was prepared from 1-((S)-(3-fluorophenyl)(hydroxy)methyl)-4-(2-(trans-4-methoxycyclohexyl)ethyl)-7-azabicyclo[2.2.1]heptane-7-carboxylate, see Example 13, Step (h) in accordance with the procedures in Example 13, Step (i).

[α]_{D}²⁰=+12.7 (c=0.32, CHCl₃)

The ¹H NMR spectrum was identical to the one for the (R) enantiomer in Example 13.

### Example 15: (R)-(3-Fluorophenyl)(4-(((4-(trifluoromethoxy)benzyl)oxy)methyl)-7-azabicyclo[2.2.1]heptan-1-yl)methanol

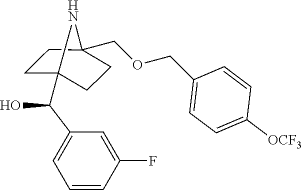 [SMILES: O[C@H](c1cccc(F)c1)C12CCC(COCc3ccc(OC(F)(F)F)cc3)(CC1)N2]

The title compound was prepared in accordance with the procedures in Example 1, using 1-(bromomethyl)-4-(trifluoromethoxy)benzene in step (d).

[α]_{D}²⁰=−15.4 (c=0.43, CHCl₃)

¹H NMR (400 MHZ, CDCl₃): δ 7.38-7.32 (2H, m) 7.31-7.24 (1H, m, overlapped with CDCl₃) 7.22-7.16 (2H, m) 7.15-7.12 (1H, m) 7.12-7.09 (1H, m) 6.99-6.92 (1H, m) 4.94 (1H, s) 4.59-4.51 (2H, m) 3.67-3.59 (2H,

### Example 16: (S)-(3-Fluorophenyl)((4-(((4-(trifluoromethoxy)benzyl)oxy)methyl)-7-azabicyclo[2.2.1]heptan-1-yl)methanol

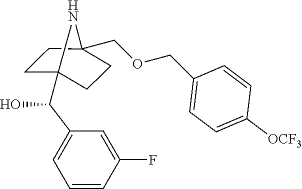 [SMILES: O[C@@H](c1cccc(F)c1)C12CCC(COCc3ccc(OC(F)(F)F)cc3)(CC1)N2]

The title compound was prepared in accordance with the procedures in Example 1, using 1-(bromomethyl)-4-(trifluoromethoxy)benzene in step (d).

[α]_{D}²⁰=−+7.1 (c=0.47, CHCl₃)

The ¹H NMR spectrum was identical to the one for the (R) enantiomer in Example 15.

### Example 17: (R)-(5-Fluoropyridin-3-yl)(4-(((4-(trifluoromethoxy)benzyl)oxy)-methyl)-7-azabicyclo[2.2.1]heptan-1-yl)methanol

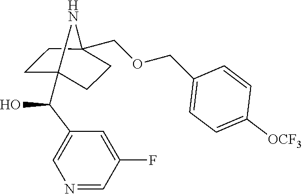 [SMILES: O[C@H](c1cncc(F)c1)C12CCC(COCc3ccc(OC(F)(F)F)cc3)(CC1)N2]

The title compound was prepared in accordance with the procedures in Example 1, using 1-(bromomethyl)-4-(trifluoromethoxy)benzene in step (d) and 3-(5-fluoropyridin-3-yl) magnesium bromide in Step (g).

[α]_{D}²⁰=−17.9 (c=0.52, CHCl₃)

¹H NMR (400 MHZ, CDCl₃): δ 8.43-8.37 (2H, m) 7.56-7.47 (1H, m) 7.40-7.32 (2H, m) 7.23-7.17 (2H, m) 5.00 (1H, s) 4.61-4.52 (2H, m) 3.68-3.60 (2H, m) 1.98-1.77 (2H, m) 1.72-1.60 (2H, m) 1.58-1.37 (3H, m) 1.19-1.07 (1H, m).

### Example 18: (S)-(5-Fluoropyridin-3-yl)(4-(((4-(trifluoromethoxy)benzyl)oxy)methyl)-7-azabicyclo[2.2.1]heptan-1-yl)methanol

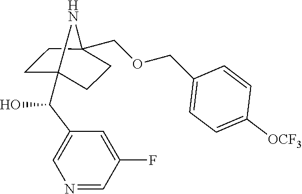 [SMILES: O[C@@H](c1cncc(F)c1)C12CCC(COCc3ccc(OC(F)(F)F)cc3)(CC1)N2]

The title compound was prepared in accordance with the procedures in Example 1, using 1-(bromomethyl)-4-(trifluoromethoxy)benzene in step (d) and 3-(5-fluoropyridin-3-yl) magnesium bromide in Step (g).

[α]_{D}²⁰=+15.0 (c=1.07, CHCl₃)

The ¹H NMR spectrum was identical to the one for the (R) enantiomer in Example 17.

### Example 19: (R)-(3-Fluorophenyl)(4-(4-(trifluoromethoxy)benzyl)-7-azabicyclo-[2.2.1]heptan-1-yl)methanol

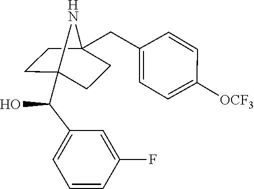 [SMILES: O[C@H](c1cccc(F)c1)C12CCC(Cc3ccc(OC(F)(F)F)cc3)(CC1)N2]

The title compound was prepared in accordance with the procedures in Example 7, using 1-(bromomethyl)-4-(trifluoromethoxy)benzene in Step (a).

[α]_{D}²⁰=−5.9 (c=0.51, CHCl₃)

¹H NMR (400 MHZ, CDCl₃) δ 7.30-7.20 (3H, m, overlapping with CHCl₃) 7.16-7.06 (4H, m) 6.98-6.92 (1H, m) 4.91 (1H, s) 2.99 (2H, s) 1.93-1.83 (1H, m) 1.63-1.55 (2H, m) 1.54-1.41 (3H, m) 1.36-1.29 (1H, m) 1.18-1.09 (1H, m).

### Example 20: (S)-(3-Fluorophenyl)(4-(4-(trifluoromethoxy)benzyl)-7-azabicyclo-[2.2.1]heptan-1-yl)methanol

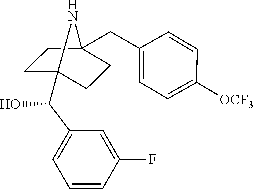 [SMILES: O[C@@H](c1cccc(F)c1)C12CCC(Cc3ccc(OC(F)(F)F)cc3)(CC1)N2]

The title compound was prepared in accordance with the procedures in Example 7, using 1-(bromomethyl)-4-(trifluoromethoxy)benzene in Step (a).

[α]_{D}²⁰=+20.6 (c=0.52, CHCl₃)

The ¹H NMR spectrum was identical to the one for the (R) enantiomer in Example 19.

### Example 21: N-Benzyl-N-(4-(4-((R)-(3-fluorophenyl)(hydroxy)methyl)-7-azabicyclo-[2.2.1]heptan-1-yl)methyl)phenyl)methanesulfonamide

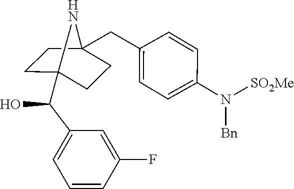 [SMILES: CS(=O)(=O)N(Cc1ccccc1)c1ccc(CC23CCC([C@H](O)c4cccc(F)c4)(CC2)N3)cc1]

### (a) tert-Butyl 1-(4-bromobenzyl)-4-(((tert-butyldimethylsilyl)oxy)methyl)-7-azabicyclo[2.2.1]heptane-7-carboxylate

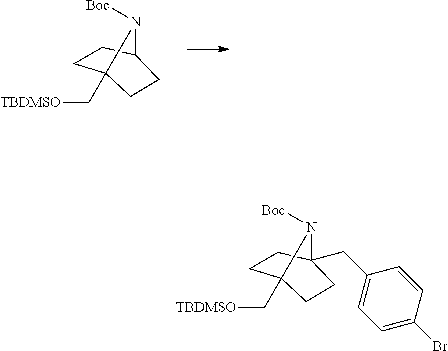 [SMILES: CC(C)(C)OC(=O)N1C2CCC1(CO[Si](C)(C)C(C)(C)C)CC2 ; CC(C)(C)OC(=O)N1C2(CO[Si](C)(C)C(C)(C)C)CCC1(Cc1ccc(Br)cc1)CC2]

The sub-title compound was prepared from tert-butyl 1-(((tert-butyldimethylsilyl)oxy)-methyl)-7-azabicyclo[2.2.1]heptane-7-carboxylate and 1-bromo-4-(bromomethyl)-benzene in accordance with the procedure in Example 7, Step (a)

### (b) tert-Butyl 1-(4-(N-benzylmethylsulfonamido)benzyl)-4-(((tert-butyldimethyl-silyl)oxy)methyl)-7-azabicyclo[2.2.1]heptane-7-carboxylate

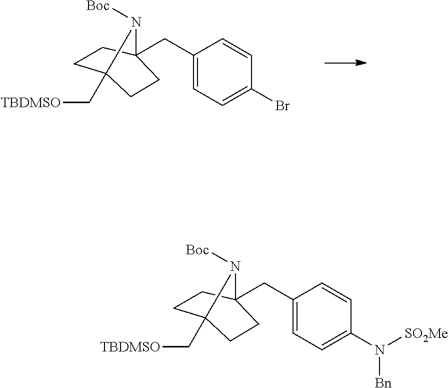 [SMILES: CC(C)(C)OC(=O)N1C2(CO[Si](C)(C)C(C)(C)C)CCC1(Cc1ccc(Br)cc1)CC2 ; CC(C)(C)OC(=O)N1C2(CO[Si](C)(C)C(C)(C)C)CCC1(Cc1ccc(N(Cc3ccccc3)S(C)(=O)=O)cc1)CC2]

A solution of tert-butyl 1-(4-bromobenzyl)-4-(((tert-butyldimethylsilyl)oxy)methyl)-7-azabicyclo[2.2.1]heptane-7-carboxylate (150 mg, 0.29 mmol) in toluene (1.5 mL) was added to a mixture of allyl palladium chloride dimer (1.6 mg, 0.03 mmol), tBuXPhos (15 mg, 0.12 mmol), K₂CO₃ (162 mg, 1.18 mM dried at 120° C. and freshly ground) and N-benzylmethylsulfonamide (109, 0.59 mmol). The vial was sealed and the mixture was stirred under Ar for 2 min at rt and at 110° C. for 16 h. The mixture was cooled to rt, diluted with CH₂Cl₂ and washed with H₂O. The aq layer was extracted with CH₂Cl₂, and the combined organic phases were washed with brine, dried (Na₂SO₄) and concentrated. The residue was purified by chromatography to give the sub-title compound (107 mg (59%).

### (c)N-Benzyl-N-(4-((4-((R)-(3-fluorophenyl)(hydroxy)methyl)-7-azabicyclo-[2.2.1]heptan-1-yl)methyl)phenyl)methanesulfonamide

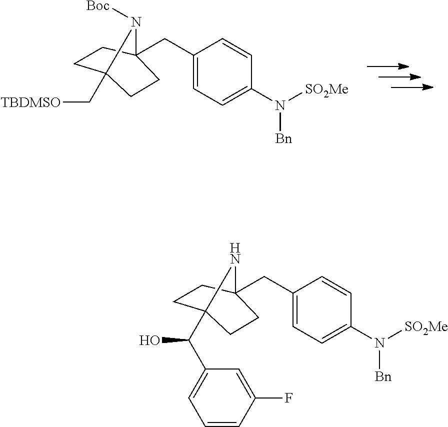 [SMILES: CC(C)(C)OC(=O)N1C2(CO[Si](C)(C)C(C)(C)C)CCC1(Cc1ccc(N(Cc3ccccc3)S(C)(=O)=O)cc1)CC2 ; CS(=O)(=O)N(Cc1ccccc1)c1ccc(CC23CCC([C@H](O)c4cccc(F)c4)(CC2)N3)cc1]

The title compound was prepared from tert-butyl 1-(4-(N-benzylmethylsulfonamido)-benzyl)-4-(((tert-butyldimethylsilyl)oxy)methyl)-7-azabicyclo[2.2.1]heptane-7-carboxylate in accordance with the procedures in Example 7, Steps (b) to (e).

[α]_{D}²⁰=−7.5 (c=0.51, CHCl₃)

¹H NMR (400 MHZ, CDCl₃) δ 7.30-7.20 (m, 6H), 7.19-7.13 (m, 4H), 7.12-7.06 (m, 2H), 6.98-6.91 (m, 1H), 4.90 (s, 1H), 4.83 (s, 2H), 2.95 (s, 3H), 2.92 (s, 2H), 1.93-1.80 (m, 1H), 1.75-1.36 (m, 7H), 1.36-1.27 (m, 1H), 1.12 (ddd, J=11.5, 8.8, 4.7 Hz, 1H).

### Example 22: N-Benzyl-N-(4-((4-((S)-(3-fluorophenyl)(hydroxy)methyl)-7-aza-bicyclo[2.2.1]heptan-1-yl)methyl)phenyl)methanesulfonamide

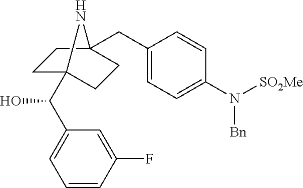 [SMILES: CS(=O)(=O)N(Cc1ccccc1)c1ccc(CC23CCC([C@@H](O)c4cccc(F)c4)(CC2)N3)cc1]

The title compound was prepared from tert-butyl 1-(4-(N-benzylmethylsulfonamido)-benzyl)-4-(((tert-butyldimethylsilyl)oxy)methyl)-7-azabicyclo[2.2.1]heptane-7-carboxylate (see Example 21, Step (b)) in accordance with the procedures in Example 7, Steps (b) to (e).

[α]_{D}²⁰=+9.3 (c=0.54, CHCl₃)

The ¹H NMR spectrum was identical to the one for the (R) enantiomer in Example 21.

### Example 23: N-Benzyl-N-(3-((4-((R)-(3-fluorophenyl)(hydroxy)methyl)-7-aza-bicyclo[2.2.1]heptan-1-yl)methyl)phenyl)methanesulfonamide

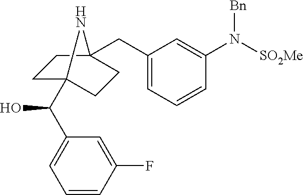 [SMILES: CS(=O)(=O)N(Cc1ccccc1)c1cccc(CC23CCC([C@H](O)c4cccc(F)c4)(CC2)N3)c1]

The title compound was prepared in accordance with the procedures in Example 21, using 1-bromo-3-(bromomethyl)benzene in Step (a).

[α]_{D}²⁰=−12.1 (c=0.69, CHCl₃)

¹H NMR (400 MHZ, CDCl₃) δ 7.30-7.26 (1H, m, overlapping CHCl₃) 7.26-7.18 (6H, m, overlapping CHCl₃) 7.17-7.13 (1H, m) 7.12-7.07 (4H, m) 6.98-6.91 (1H, m) 4.92 (1H, s) 4.88-4.78 (2H, m) 2.96 (3H, s) 2.95-2.89 (2H, m) 2.61-2.20 (2H, br s) 1.91-1.80 (1H, m) 1.64-1.54 (1H, m) 1.54-1.40 (2H, m) 1.40-1.22 (3H, m) 1.12-1.04 (1H, m).

### Example 24: N-Benzyl-N-(3-((4-((S)-(3-fluorophenyl)(hydroxy)methyl)-7-aza-bicyclo[2.2.1]heptan-1-yl)methyl)phenyl)methanesulfonamide

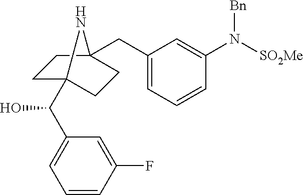 [SMILES: CS(=O)(=O)N(Cc1ccccc1)c1cccc(CC23CCC([C@@H](O)c4cccc(F)c4)(CC2)N3)c1]

The title compound was prepared in accordance with the procedures in Example 21, using 1-bromo-3-(bromomethyl)benzene in Step (a).

[α]_{D}²⁰=+10.3 (c=0.65, CHCl₃)

The ¹H NMR spectrum was identical to the one for the (R) enantiomer in Example 23.

### Example 25: N-(4-((4-((R)-(3-Fluorophenyl)(hydroxy)methyl)-7-azabicyclo[2.2.1]-heptan-1-yl)methyl)phenyl)methanesulfonamide

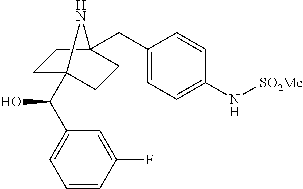 [SMILES: CS(=O)(=O)Nc1ccc(CC23CCC([C@H](O)c4cccc(F)c4)(CC2)N3)cc1]

A mixture of N-benzyl-N-(4-((4-((R)-(3-fluorophenyl)(hydroxy)methyl)-7-azabicyclo-[2.2.1]heptan-1-yl)methyl)phenyl)methanesulfonamide (see Example 21) (10 mg, 20 μmol), Pd (OH)₂ (20% on carbon, nominally 50% H₂O, 17 mg, 12 μmol,) and AcOH (0.4 mL) was hydrogenated at ambient temperature and pressure for 5 h. The mixture was filtered through a pad of Celite, which was washed with MeOH. The combined filtrates were concentrated and the residue triturated with Et₂O. The solids were collected and dried to give the title compound (8 mg, 98%).

[α]_{D}²⁰=−5.0 (c=1.00, CHCl₃)

¹H NMR (400 MHZ, CD3OD) δ 7.35-7.27 (m, 1H), 7.22-7.07 (m, 6H), 7.00-6.93 (m, 1H), 4.90 (s, 1H), 3.05-2.88 (m, 5H), 1.99-1.86 (m, 1H), 1.74-1.42 (m, 6H), 1.38-1.14 (m, 1H).

### Example 26: N-(4-((4-((S)-(3-Fluorophenyl)(hydroxy)methyl)-7-azabicyclo[2.2.1]-heptan-1-yl)methyl)phenyl)methanesulfonamide

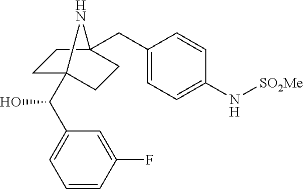 [SMILES: CS(=O)(=O)Nc1ccc(CC23CCC([C@@H](O)c4cccc(F)c4)(CC2)N3)cc1]

The title compound was prepared from N-benzyl-N-(4-((4-((S)-(3-fluorophenyl)-(hydroxy)methyl)-7-azabicyclo[2.2.1]heptan-1-yl)methyl)phenyl)methanesulfonamide (see Example 22) in accordance with the procedure in Example 25.

[α]_{D}²⁰=+10.9 (c=0.90 CHCl₃)

The ¹H NMR spectrum was identical to the one for the (R) enantiomer in Example 25.

### Example 27: N-(3-((4-((R)-(3-Fluorophenyl)(hydroxy)methyl)-7-azabicyclo-[2.2.1]heptan-1-yl)methyl)phenyl)methanesulfonamide

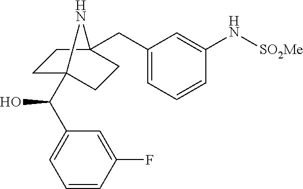 [SMILES: CS(=O)(=O)Nc1cccc(CC23CCC([C@H](O)c4cccc(F)c4)(CC2)N3)c1]

The title compound was prepared from N-benzyl-N-(3-((4-((R)-(3-fluorophenyl)-(hydroxy)methyl)-7-azabicyclo[2.2.1]heptan-1-yl)methyl)phenyl)methanesulfonamide (see Example 23) in accordance with the procedure in Example 25.

[α]_{D}²⁰=−6.7 (c=1.04 CHCl₃)

¹H NMR (400 MHZ, CDCl₃) δ 7.38-7.33 (1H, m) 7.32-7.27 (2H, m) 7.25-7.17 (1H, m) 7.10-7.02 (2H, m) 6.97-6.89 (2H, m) 5.35 (1H, s) 3.27 (2H, s) 3.00 (3H, s)

### Example 28: N-(3-((4-((S)-(3-Fluorophenyl)(hydroxy)methyl)-7-azabicyclo[2.2.1]-heptan-1-yl)methyl)phenyl)methanesulfonamide

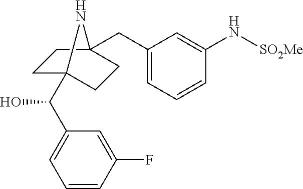 [SMILES: CS(=O)(=O)Nc1cccc(CC23CCC([C@@H](O)c4cccc(F)c4)(CC2)N3)c1]

The title compound was prepared from N-benzyl-N-(3-((4-((S)-(3-fluoro-phenyl)(hydroxy)methyl)-7-azabicyclo[2.2.1]heptan-1-yl)methyl)phenyl) methane-sulfonamide (see Example 24) in accordance with the procedure in Example 25.

[α]_{D}²⁰=+7.8 (c=1.03 CHCl₃)

The ¹H NMR spectrum was identical to the one for the (R) enantiomer in Example 27.

### Example 29: (R)-(4-Cinnamyl-7-azabicyclo[2.2.1]heptan-1-yl)(5-fluoropyridin-3-yl)-methanol

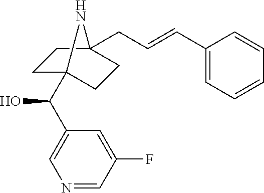 [SMILES: O[C@H](c1cncc(F)c1)C12CCC(C/C=C/c3ccccc3)(CC1)N2]

### (a) tert-Butyl 1-(((tert-butyldimethylsilyl)oxy)methyl)-4-cinnamyl-7-azabicyclo-[2.2.1]heptane-7-carboxylate

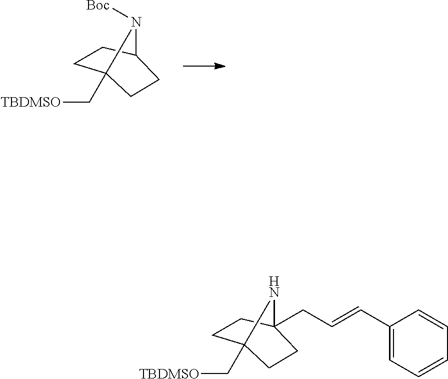 [SMILES: CC(C)(C)OC(=O)N1C2CCC1(CO[Si](C)(C)C(C)(C)C)CC2 ; CC(C)(C)[Si](C)(C)OCC12CCC(C/C=C/c3ccccc3)(CC1)N2]

sec-BuLi (1.3 M in cyclohexane, 0.73 mL, 0.94 mmol) was added dropwise to a solution of tert-butyl 1-(((tert-butyldimethylsilyl)oxy)methyl)-7-azabicyclo[2.2.1]heptane-7-carboxylate, see Example 1, Step (b), (215 mg, 0.63 mmol) and TMEDA (142 μL, 0.94 mmol) in Et₂O (3.0 mL) at −78° C. The mixture was stirred at −78° C. for 1 h and a solution of CuCN·2LiCl (0.3 M in anhydrous degassed THF, 2.1 mL, 0.63 mmol) was added dropwise and stirring was continued at −78° C. for 1 h. Cinnamyl bromide (273 mg, 1.38 mmol) in THF (0.5 mL) was added dropwise and the mixture was allowed to warm to rt and stirred for 18 h. NH₄Cl (aq, sat) was added and the mixture was extracted with CH₂Cl₂ and the combined organic phases were washed with brine, dried (Na₂SO₄) and concentrated. The residue was purified by chromatography to give the sub-title compound (165 mg, 57%).

### (b) (R)-(4-Cinnamyl-7-azabicyclo[2.2.1]heptan-1-yl)(5-fluoropyridin-3-yl)methanol

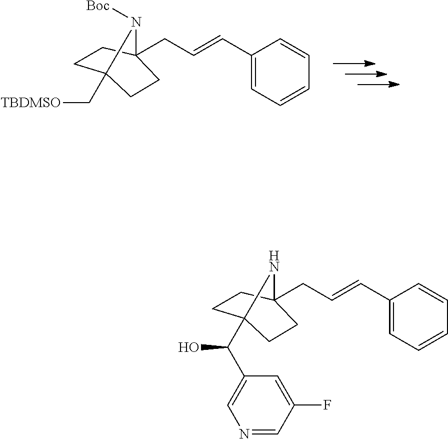 [SMILES: CC(C)(C)OC(=O)N1C2(C/C=C/c3ccccc3)CCC1(CO[Si](C)(C)C(C)(C)C)CC2 ; O[C@H](c1cncc(F)c1)C12CCC(C/C=C/c3ccccc3)(CC1)N2]

The title compound was prepared from tert-butyl 1-(((tert-butyldimethylsilyl)oxy)methyl)-4-cinnamyl-7-azabicyclo[2.2.1]heptane-7-carboxylate in accordance with the procedures in Example 7, Steps (b) to (e), using 3-(5-fluoropyridin-3-yl) magnesium bromide in Step (d).

[α]_{D}²⁰=−11.1 (c=0.21, CHCl₃)

¹H NMR (400 MHZ, CDCl₃) δ 8.40-8.35 (2H, m) 7.54-7.48 (1H, m) 7.39-7.34 (2H, m) 7.33-7.27 (2H, m) 7.25-7.19 (1H, m) 6.52-6.45 (1H, m) 6.29-6.18 (1H, m) 5.09 (1H, s) 2.65 (2H, d, J=7.3 Hz) 2.11-1.99 (2H, m) 1.87-1.76 (2H, m) 1.74-1.68 (1H, m) 1.65-1.56 (1H, m) 1.46-1.40 (1H, m) 1.19-1.10 (1H, m).

### Example 30: (S)-(4-Cinnamyl-7-azabicyclo[2.2.1]heptan-1-yl)(5-fluoropyridin-3-yl)-methanol

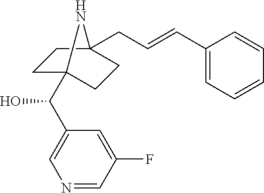 [SMILES: O[C@@H](c1cncc(F)c1)C12CCC(C/C=C/c3ccccc3)(CC1)N2]

The title compound was prepared from tert-butyl 1-(((tert-butyldimethylsilyl)oxy)methyl)-4-cinnamyl-7-azabicyclo[2.2.1]heptane-7-carboxylate in accordance with the procedures in Example 7, Steps (b) to (e), using 3-(5-fluoropyridin-3-yl) magnesium bromide in Step (d).

[α]_{D}²⁰=+32.2 (c=0.24, CHCl₃)

The ¹H NMR spectrum was identical to the one for the (R) enantiomer in Example 29.

### Example 31: (R)-(4-Cinnamyl-7-azabicyclo[2.2.1]heptan-1-yl)(3-fluorophenyl)methanol

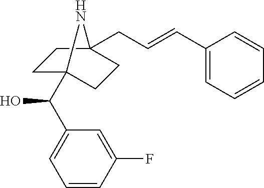 [SMILES: O[C@H](c1cccc(F)c1)C12CCC(C/C=C/c3ccccc3)(CC1)N2]

The title compound was prepared from tert-butyl 1-(((tert-butyldimethylsilyl)oxy)methyl)-4-cinnamyl-7-azabicyclo[2.2.1]heptane-7-carboxylate in accordance with the procedures in Example 7, Steps (b) to (e).

[α]_{D}²⁰=−4.7 (c=0.85, CHCl₃)

¹H NMR (400 MHZ, CDCl₃) δ 7.39-7.34 (2H, m) 7.34-7.26 (3H, m) 7.25-7.19 (1H, m) 7.15-7.10 (2H, m) 6.98-6.92 (1H, m) 6.50-6.43 (1H, m) 6.30-6.21 (1H, m) 4.94 (1H, s) 2.57 (2H, dd, J=7.4, 0.9 Hz) 1.97-1.85 (1H, m) 1.70-1.56 (3H, m) 1.56-1.48 (2H, m) 1.38-1.31 (1H, m) 1.19-1.10 (1H, m).

### Example 32: (S)-(4-Cinnamyl-7-azabicyclo[2.2.1]heptan-1-yl)(3-fluorophenyl)methanol

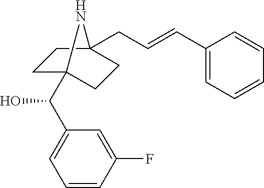 [SMILES: O[C@@H](c1cccc(F)c1)C12CCC(C/C=C/c3ccccc3)(CC1)N2]

The title compound was prepared from tert-butyl 1-(((tert-butyldimethylsilyl)oxy)methyl)-4-cinnamyl-7-azabicyclo[2.2.1]heptane-7-carboxylate in accordance with the procedures in Example 7, Steps (b) to (e).

[α]_{D}²⁰=+6.3 (c=0.89, CHCl₃)

The ¹H NMR spectrum was identical to the one for the (R) enantiomer in Example 31.

### Example 33: (R)-(5-Fluoropyridin-3-yl)(4-(3-phenylpropyl)-7-azabicyclo[2.2.1]heptan-1-yl)methanol hydrochloride

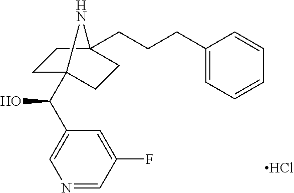 [SMILES: O[C@H](c1cncc(F)c1)C12CCC(CCCc3ccccc3)(CC1)N2 ; Cl]

A mixture of (R)-(4-cinnamyl-7-azabicyclo[2.2.1]heptan-1-yl)(5-fluoropyridin-3-yl)-methanol (13.8 mg, 40 μmol), Pd (10% on carbon, 3.7 mg, 3.5 μmol) and iPrOH (1 mL) was hydrogenated at ambient temperature and pressure for 20 h. The mixture was filtered through a pad of Celite, which was washed with iPrOH. The combined filtrates were concentrated and the residue was treated with HCl (2 M in Et₂O, 35 μL, 70 μmol). The solids were collected and dried to give the title compound (13 mg, 76%).

[α]_{D}²⁰=−12.0 (c=0.33, CHCl₃)

¹H NMR (400 MHZ, CD3OD) δ 8.78-8.54 (2H, m) 8.11-8.03 (1H, m) 7.31-7.23 (2H, m) 7.23-7.13 (3H, m) 5.22 (1H, s) 2.70 (2H, t, J=7.2 Hz) 2.39-2.26 (1H, m) 2.24-2.10 (1H, m) 2.03-1.80 (6H, m) 1.79-1.68 (2H, m) 1.68-1.59 (1H, m) 1.52-1.42 (1H, m).

### Example 34: (S)-(5-Fluoropyridin-3-yl)(4-(3-phenylpropyl)-7-azabicyclo[2.2.1]heptan-1-yl)methanol hydrochloride

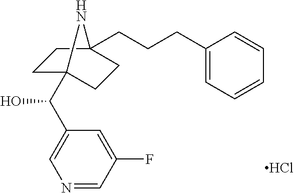 [SMILES: O[C@@H](c1cncc(F)c1)C12CCC(CCCc3ccccc3)(CC1)N2 ; Cl]

The title compound was prepared from(S)-(4-cinnamyl-7-azabicyclo[2.2.1]heptan-1-yl)(5-fluoropyridin-3-yl)methanol in accordance with the procedure in Example 33.

[α]_{D}²⁰=+17.9 (c=0.73, MeOH)

The ¹H NMR spectrum was identical to the one for the (R) enantiomer in Example 33.

### Example 35: (R)-(3-Fluorophenyl)(4-(3-phenylpropyl)-7-azabicyclo[2.2.1]heptan-1-yl)methanol hydrochloride

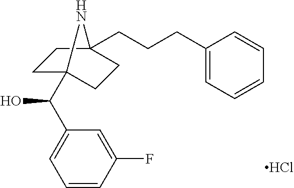 [SMILES: O[C@H](c1cccc(F)c1)C12CCC(CCCc3ccccc3)(CC1)N2 ; Cl]

The title compound was prepared from tert-butyl 1-(((tert-butyldimethylsilyl)oxy)-methyl)-4-cinnamyl-7-azabicyclo[2.2.1]heptane-7-carboxylate, see Example 29, Step (a), in accordance with the procedures in Example 7, Steps (b) to (e), using 3-fluorophenylmagnesium bromide in Step (d).

[α]_{D}²⁰=−11.8 (c=0.42, MeOH)

¹H NMR (400 MHZ, CD3OD) δ 7.41-7.35 (1H, m) 7.30-7.24 (2H, m) 7.22-7.14 (5H, m) 7.09-7.02 (1H, m) 4.99 (1H, s) 2.69 (2H, t, J=7.3 Hz) 2.41-2.30 (1H, m) 2.14-2.03 (1H, m) 1.98-1.79 (6H, m) 1.79-1.64 (2H, m) 1.60-1.50 (1H, m) 1.46-1.37 (1H, m).

### Example 36: (S)-(3-Fluorophenyl)(4-(3-phenylpropyl)-7-azabicyclo[2.2.1]heptan-1-yl)methanol hydrochloride

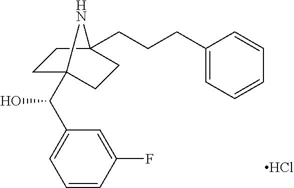 [SMILES: O[C@@H](c1cccc(F)c1)C12CCC(CCCc3ccccc3)(CC1)N2 ; Cl]

The title compound was prepared from tert-butyl 1-(((tert-butyldimethylsilyl)oxy)methyl)-4-cinnamyl-7-azabicyclo[2.2.1]heptane-7-carboxylate, see Example 29, Step (a), in accordance with the procedures in Example 7, Steps (b) to (e), using 3-fluorophenylmagnesium bromide in Step (d).

[α]_{D}²⁰=+13.4 (c=0.97, MeOH).

The ¹H NMR spectrum was identical to the one for the (R) enantiomer in Example 35.

### Example 37: (R)-4-(4-Chlorophenethyl)-7-azabicyclo[2.2.1]heptan-1-yl)-(3-fluorophenyl)methanol acetate

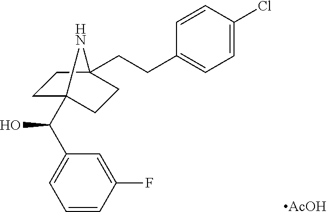 [SMILES: "·AcOH" ; O[C@H](c1cccc(F)c1)C12CCC(CCc3ccc(Cl)cc3)(CC1)N2]

### (a) tert-Butyl 1-(((tert-butyldimethylsilyl)oxy)methyl)-4-((4-chlorophenyl) ethynyl)-7-azabicyclo[2.2.1]heptane-7-carboxylate

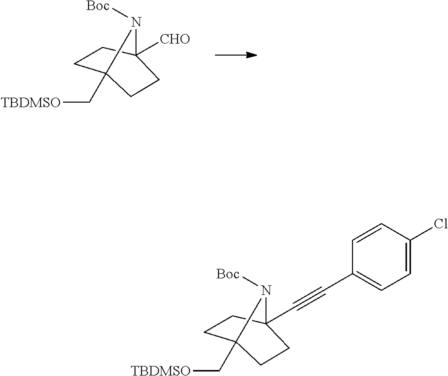 [SMILES: CC(C)(C)OC(=O)N1C2(C=O)CCC1(CO[Si](C)(C)C(C)(C)C)CC2 ; CC(C)(C)OC(=O)N1C2(C#Cc3ccc(Cl)cc3)CCC1(CO[Si](C)(C)C(C)(C)C)CC2]

Cs₂CO₃ (1.09 g, 3.36 mmol) was added to an ice-cooled mixture of tert-butyl 1-(((tert-butyldimethylsilyl)oxy)methyl)-4-formyl-7-azabicyclo[2.2.1]heptane-7-carboxylate, see Example 13, Step (c), (434 mg, 1.17 mmol), 1-chloro-4-iodobenzene (200 mg, 0.84 mmol), dimethyl (1-diazo-2-oxopropyl)phosphonate (322 mg, 1.68 mmol) and MeOH (5 mL). The mixture was stirred at 30° C. for 4 h, and a mixture of Pd (OAc)₂ (7.53 mg, 0.034 mmol), Xphos (32 mg, 0.067 mmol) and MeOH (3 mL) was added. The mixture was stirred at 40° C. for 12 h, allowed to cool to rt, diluted with H₂O and extracted with CH₂Cl₂. The combined organic phases were washed with brine, dried (Na₂SO₄) and concentrated. The residue was purified by chromatography to give the sub-title compound (188 mg, 47%)

### (b) (R)-(4-((4-Chlorophenyl) ethynyl)-7-azabicyclo[2.2.1]heptan-1-yl)(3-fluoro-phenyl)methanol

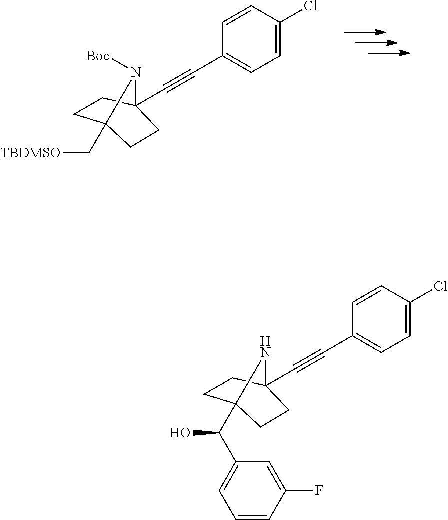 [SMILES: CC(C)(C)OC(=O)N1C2(C#Cc3ccc(Cl)cc3)CCC1(CO[Si](C)(C)C(C)(C)C)CC2 ; O[C@H](c1cccc(F)c1)C12CCC(C#Cc3ccc(Cl)cc3)(CC1)N2]

The title compound was prepared from tert-butyl 1-(((tert-butyldimethylsilyl)oxy)methyl)-4-((4-chlorophenyl) ethynyl)-7-azabicyclo[2.2.1]heptane-7-carboxylate in accordance with the procedures in Example 7, Steps (b) to (e).

[α]_{D}²⁰=−14.2 (c=1.01, CHCl₃)

¹H NMR (400 MHz, CDCl₃) δ 7.38-7.33 (2H, m) 7.32-7.27 (2H, m) 7.27-7.24 (1H, m) 7.16-7.10 (2H, m) 7.01-6.94 (1H, m) 4.97 (1H, s) 2.05-1.91 (4H, m) 1.91-1.83 (1H, m) 1.79-1.68 (1H, m) 1.45-1.36 (1H, m) 1.30-1.21 (1H, m).

### (c) (R)-(4-(4-Chlorophenethyl)-7-azabicyclo[2.2.1]heptan-1-yl)(3-fluorophenyl)-methanol acetate

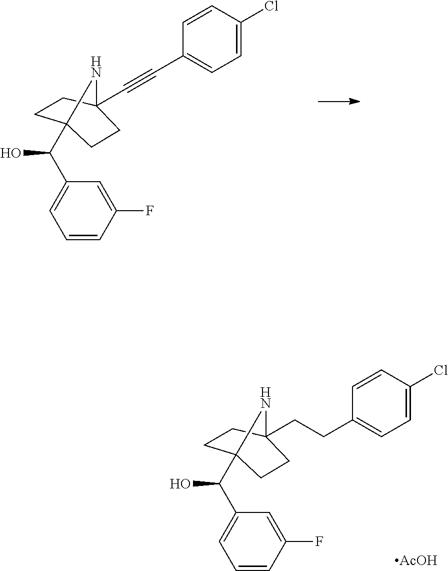

A mixture f (R)-(4-((4-chlorophenyl) ethynyl)-7-azabicyclo[2.2.1]heptan-1-yl)-(3-fluorophenyl)methanol (25 mg, 72 μmol), PtO₂×H₂O (2.8 mg, 11 μmol) and MeOH (0.5 mL) was hydrogenated at ambient temperature and pressure for 20 h. The mixture was filtered through a pad of Celite, which was washed with MeOH, and the combined filtrates were filtered through a syringe filter (PTFE, 0.2 μm) and concentrated. The residue was dissolved in H₂O/AcOH/MeCN (0.1 mL/0.1 mL/0.1 mL) and purified by preparative reversed phase HPLC (XBridge C18 OBD prep column, 130A, 5 μm; 10 mm×150 mm) using an elution gradient from 10% to 95% MeCN in 0.1% AcOH in H₂O to give the title compound (10.7 mg, 36%).

[α]_{D}²⁰=−4.7 (c=0.85, CHCl₃)

¹H NMR (300 MHZ, CDCl₃) δ 7.25-7.19 (1H, m) 7.18-7.13 (2H, m) 7.12-7.03 (4H, m) 6.99-6.90 (1H, m) 5.13 (1H, s) 2.71-2.60 (2H, m) 2.46-2.31 (1H, m) 2.23-2.12 (2H, m) 2.01-1.83 (3H, m) 1.77-1.62 (2H, m) 1.50-1.39 (1H, m) 1.31-1.19 (1H, m)

### Example 38: (S)-(4-(4-Chlorophenethyl)-7-azabicyclo[2.2.1]heptan-1-yl)(3-fluoro-phenyl)methanol acetate

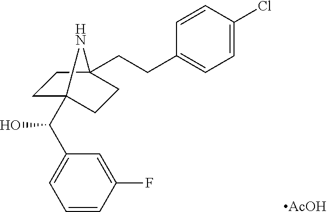 [SMILES: "·AcOH" ; O[C@@H](c1cccc(F)c1)C12CCC(CCc3ccc(Cl)cc3)(CC1)N2]

The title compound was prepared from tert-butyl 1-(((tert-butyldimethylsilyl)oxy)methyl)-4-((4-chlorophenyl) ethynyl)-7-azabicyclo[2.2.1]heptane-7-carboxylate in accordance with the procedure in Example 37, Steps (b) and (c).

[α]_{D}²⁰=+7.5 (c=0.53, CHCl₃)

The ¹H NMR spectrum was identical to the one for the (R) enantiomer in Example 37.

### Example 39: (R)-(3-Fluorophenyl)(4-(4-methoxyphenethyl)-7-azabicyclo[2.2.1]heptan-1-yl)methanol acetate

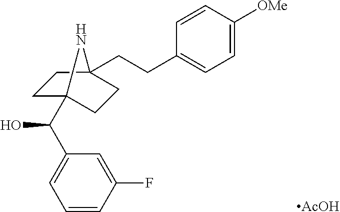 [SMILES: "·AcOH" ; COc1ccc(CCC23CCC([C@H](O)c4cccc(F)c4)(CC2)N3)cc1]

### (a) tert-Butyl 1-(((tert-butyldimethylsilyl)oxy)methyl)-4-ethynyl-7-azabicyclo-[2.2.1]heptane-7-carboxylate

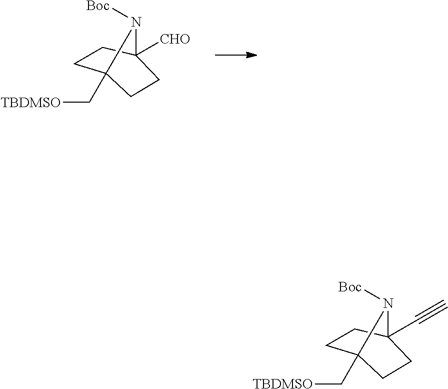 [SMILES: CC(C)(C)OC(=O)N1C2(C=O)CCC1(CO[Si](C)(C)C(C)(C)C)CC2 ; C#CC12CCC(CO[Si](C)(C)C(C)(C)C)(CC1)N2C(=O)OC(C)(C)C]

K₂CO₃ (0.69 g, 5.0 mmol) was added to a mixture of tert-butyl 1-(((tert-butyldimethyl-silyl)oxy)methyl)-4-formyl-7-azabicyclo[2.2.1]heptane-7-carboxylate, see Example 13, Step (c), (530 mg, 1.43 mmol), dimethyl (1-diazo-2-oxopropyl)phosphonate (0.69 g, 3.58 mmol) and MeOH (3 mL) at rt and the mixture was stirred at rt for 21 h. NH₄Cl (aq, sat) was added and a mixture was extracted with EtOAc. The combined organic phases were washed with brine, dried (Na₂SO₄) and concentrated. The residue was purified by chromatography to give the sub-title compound (0.32 g, 61%)

### (b) tert-Butyl 1-(((tert-butyldimethylsilyl)oxy)methyl)-4-((4-methoxyphenyl) ethynyl)-7-azabicyclo[2.2.1]heptane-7-carboxylate

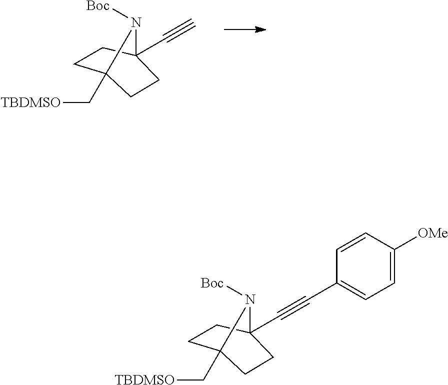 [SMILES: C#CC12CCC(CO[Si](C)(C)C(C)(C)C)(CC1)N2C(=O)OC(C)(C)C ; COc1ccc(C#CC23CCC(CO[Si](C)(C)C(C)(C)C)(CC2)N3C(=O)OC(C)(C)C)cc1]

A solution of tert-butyl 1-(((tert-butyldimethylsilyl)oxy)methyl)-4-ethynyl-7-aza-bicyclo[2.2.1]heptane-7-carboxylate (0.37 g, 1.0 mmol) in degassed Et₃N (4.5 mL) was added dropwise to a degassed stirred mixture of 4-iodoanisole (0.31 g, 1.32 mmol), Pd(Ph₃)₂Cl₂ (14 mg, 20 μmol), CuI (7.7 mg, 40 μmol) and Et₃N (4.5 mL) at rt and the mixture was stirred at rt for 20 h. NH₄Cl (aq, sat) was added and a mixture was extracted with EtOAc. The combined organic phases were washed with brine, dried (Na₂SO₄) and concentrated. The residue was purified by chromatography to give the sub-title compound (507 mg, 93%)

### (c) (R)-(3-Fluorophenyl)(4-((4-methoxyphenyl) ethynyl)-7-azabicyclo[2.2.1]heptan-1-yl)methanol acetate

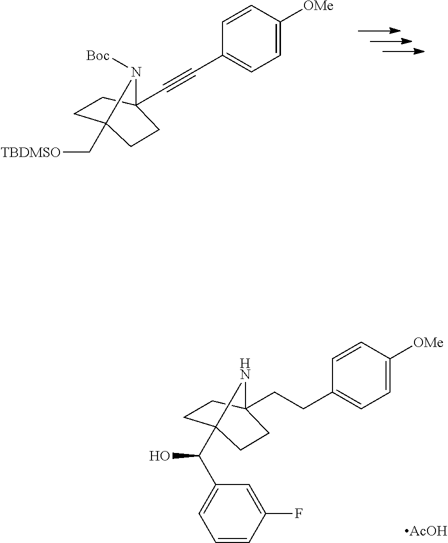 [SMILES: COc1ccc(C#CC23CCC(CO[Si](C)(C)C(C)(C)C)(CC2)N3C(=O)OC(C)(C)C)cc1 ; "·AcOH" ; COc1ccc(CCC23CCC([C@H](O)c4cccc(F)c4)(CC2)N3)cc1]

The title compound was prepared from tert-butyl 1-(((tert-butyldimethylsilyl)oxy)-methyl)-4-((4-methoxyphenyl) ethynyl)-7-azabicyclo[2.2.1]heptane-7-carboxylate in accordance with the procedure in Example 7, Steps (b) to (e), followed by the procedure in Example 37, Step (c).

[α]_{D}²⁰=−11.0 (c=0.46, CHCl₃)

¹H NMR (400 MHZ, CDCl₃) δ 7.25-7.18 (1H, m) 7.13-7.03 (4H, m) 6.96-6.89 (1H, m) 6.77-6.72 (2H, m) 5.14 (1H, s) 3.75 (3H, s) 2.69-2.56 (2H, m) 2.44-2.33 (1H, m) 2.26-2.10 (2H, m) 2.01-1.83 (3H, m) 1.76-1.63 (2H, m) 1.47-1.37 (1H, m) 1.28-1.19 (1H, m).

### Example 40: (S)-(3-Fluorophenyl)(4-(4-methoxyphenethyl)-7-azabicyclo[2.2.1]heptan-1-yl)methanol acetate

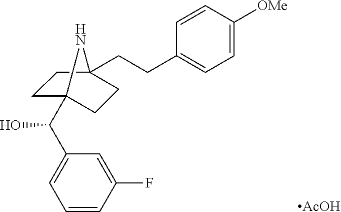 [SMILES: "·AcOH" ; COc1ccc(CCC23CCC([C@@H](O)c4cccc(F)c4)(CC2)N3)cc1]

The title compound was prepared from tert-butyl 1-(((tert-butyldimethylsilyl)oxy)methyl)-4-((4-methoxyphenyl) ethynyl)-7-azabicyclo[2.2.1]heptane-7-carboxylate, see Example 39, Step (b), in accordance with the procedure in Example 7, Steps (b) to (e), followed by the procedure in Example 37, Step (c).

[α]_{D}²⁰=+6.4 (c=0.47, CHCl₃)

The ¹H NMR spectrum was identical to the one for the (R) enantiomer in Example 39.

### Example 41: (R)-(5-Fluoropyridin-3-yl)(4-(4-methoxyphenethyl)-7-azabicyclo-[2.2.1]heptan-1-yl)methanol dihydrochloride

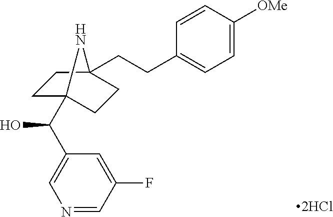 [SMILES: COc1ccc(CCC23CCC([C@H](O)c4cncc(F)c4)(CC2)N3)cc1 ; "·2HCl"]

The title compound was prepared from tert-butyl 1-(((tert-butyldimethylsilyl)oxy)methyl)-4-((4-methoxyphenyl) ethynyl)-7-azabicyclo[2.2.1]heptane-7-carboxylate, see Example 39, Step (b), in accordance with the procedure in Example 7, steps (b) to (e), using (5-fluoropyridin-3-yl) magnesium bromide in Step (d), followed by the procedure in Example 37, Step (c). Purification was performed by preparative reversed-phase HPLC (Atlantis C₁₈ T3 OBD prep column, 100A, 5 μm; 10 mm×150 mm) using an elution gradient from 5% to 95% MeCN in 0.05% AcOH. The appropriate fractions were concentrated and the residue suspended in Et₂O. Addition of HCl (2 M in Et₂O) gave a precipitate that was collected and dried to give the title compound.

[α]_{D}²⁰=−14.6 (c=0.48, MeOH)

¹H NMR (400 MHZ, CD3OD) δ 8.75-8.60 (2H, m) 8.12-8.06 (1H, m) 7.18-7.12 (2H, m) 6.88-6.82 (2H, m) 5.25 (1H, s) 3.76 (3H, s) 2.74-2.61 (2H, m) 2.41-2.30 (1H, m) 2.27-2.11 (3H, m) 2.07-1.85 (4H, m) 1.72-1.62 (1H, m) 1.55-1.44 (1H, m).

### Example 42: (S)-(5-Fluoropyridin-3-yl)(4-(4-methoxyphenethyl)-7-azabicyclo-[2.2.1]heptan-1-yl)methanol dihydrochloride

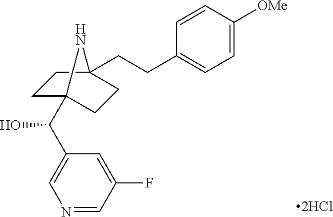 [SMILES: COc1ccc(CCC23CCC([C@@H](O)c4cncc(F)c4)(CC2)N3)cc1 ; "·2HCl"]

The title compound was prepared from tert-butyl 1-(((tert-butyldimethylsilyl)oxy)methyl)-4-((4-methoxyphenyl) ethynyl)-7-azabicyclo[2.2.1]heptane-7-carboxylate in accordance with the procedure in Example 41.

[α]_{D}²⁰=+21.5 (c=0.56, MeOH)

The ¹H NMR spectrum was identical to the one for the (R) enantiomer in Example 41.

### Example 43: (R)-(3-Fluorophenyl)(4-(4-(trifluoromethoxy) phenethyl)-7-azabicyclo-[2.2.1]heptan-1-yl)methanol hydrochloride

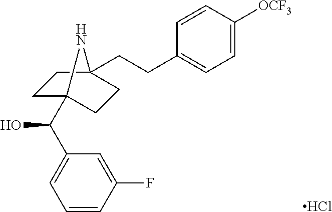 [SMILES: O[C@H](c1cccc(F)c1)C12CCC(CCc3ccc(OC(F)(F)F)cc3)(CC1)N2 ; Cl]

The title compound is prepared from tert-butyl 1-(((tert-butyldimethylsilyl)oxy)methyl)-4-((4-(trifluoromethoxy)phenyl) ethynyl)-7-azabicyclo[2.2.1]heptane-7-carboxylate (prepared from 1-iodo-4-trifluoromethoxybenzene in accordance with the procedure in Example 39, Step (b)) in accordance with the procedure in Example 41.

### Example 44: (S)-(3-Fluorophenyl)(4-(4-(trifluoromethoxy) phenethyl)-7-azabicyclo-[2.2.1]heptan-1-yl)methanol hydrochloride

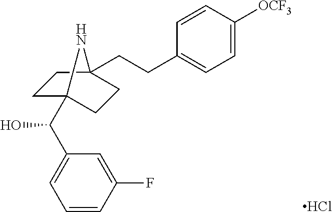 [SMILES: O[C@@H](c1cccc(F)c1)C12CCC(CCc3ccc(OC(F)(F)F)cc3)(CC1)N2 ; Cl]

The title compound was prepared from tert-butyl 1-(((tert-butyldimethylsilyl)oxy)methyl)-4-((4-(trifluoromethoxy)phenyl) ethynyl)-7-azabicyclo[2.2.1]heptane-7-carboxylate (prepared from 1-iodo-4-trifluoromethoxybenzene in accordance with the procedure in Example 39, Step (b)) in accordance with the procedure in Example 41.

¹H NMR (400 MHZ, CD3OD) δ 7.44-7.37 (1H, m) 7.37-7.32 (2H, m) 7.26-7.16 (4H, m) 7.11-7.04 (1H, m) 5.02 (1H, s) 2.83-2.68 (2H, m) 2.45-2.33 (1H, m) 2.27-2.09 (3H, m) 2.05-1.93 (3H, m) 1.93-1.83 (1H, m) 1.67-1.58 (1H, m), 1.51-1.42 (1H, m).

### Example 45: (R)-(5-Fluoropyridin-3-yl)(4-(4-(trifluoromethoxy) phenethyl)-7-aza-bicyclo[2.2.1]heptan-1-yl)methanol dihydrochloride

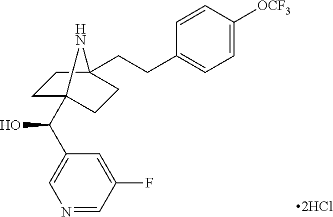 [SMILES: O[C@H](c1cncc(F)c1)C12CCC(CCc3ccc(OC(F)(F)F)cc3)(CC1)N2 ; "·2HCl"]

The title compound is prepared from tert-buty 1-(((tert-butyldimethylsilyl)oxy)methyl)-4-((4-(trifluoromethoxy)phenyl) ethynyl)-7-azabicyclo[2.2.1]heptane-7-carboxylate (prepared from 1-iodo-4-trifluoromethoxybenzene in accordance with the procedure in Example 39, Step (b)) in accordance with the procedure in Example 41.

### Example 46: (S)-(5-Fluoropyridin-3-yl)(4-(4-(trifluoromethoxy) phenethyl)-7-aza-bicyclo[2.2.1]heptan-1-yl)methanol dihydrochloride

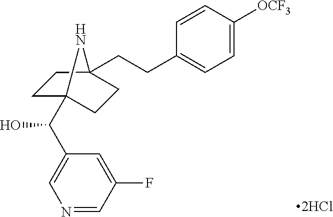 [SMILES: O[C@@H](c1cncc(F)c1)C12CCC(CCc3ccc(OC(F)(F)F)cc3)(CC1)N2 ; "·2HCl"]

The title compound was prepared from tert-butyl 1-(((tert-butyldimethylsilyl)oxy)methyl)-4-((4-(trifluoromethoxy)phenyl) ethynyl)-7-azabicyclo[2.2.1]heptane-7-carboxylate in accordance with the procedure in Example 47.

¹H NMR (300 MHZ, CD3OD) δ 8.79-8.63 (2H, m) 8.18-8.09 (1H, m) 7.40-7.31 (2H, m) 7.25-7.17 (2H, m) 5.27 (1H, s) 2.83-2.71 (2H, m) 2.46-2.30 (1H, m) 2.30-2.12 (3H, m) 2.11-1.83 (4H, m) 1.77-1.63 (1H, m) 1.58-1.44 (1H, m).

### Example 47: (R)-(2-Fluorophenyl)(4-(2-(pyridin-3-yl)ethyl)-7-azabicyclo[2.2.1]heptan-1-yl)methanol dihydrochloride

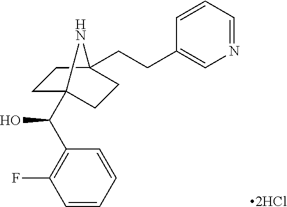 [SMILES: O[C@H](c1ccccc1F)C12CCC(CCc3cccnc3)(CC1)N2 ; "·2HCl"]

The title compound is prepared from tert-butyl 1-(((tert-butyldimethylsilyl)oxy)methyl)-4-(pyridin-3-ylethynyl)-7-azabicyclo[2.2.1]heptane-7-carboxylate (prepared from 3-iodopyridine in accordance with the procedure in Example 39, Step (b)) in accordance with the procedure in Example 7, steps (b) to (e), using 2-fluorophenylmagnesium bromide in Step (d), followed by the procedure in Example 37, Step (c).

### Example 48: (S)-(2-Fluorophenyl)(4-(2-(pyridin-3-yl)ethyl)-7-azabicyclo[2.2.1]heptan-1-yl)methanol dihydrochloride

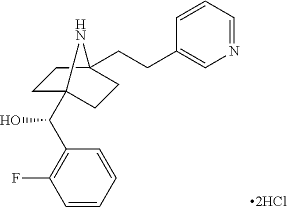 [SMILES: O[C@@H](c1ccccc1F)C12CCC(CCc3cccnc3)(CC1)N2 ; "·2HCl"]

The title compound is prepared from tert-butyl 1-(((tert-butyldimethylsilyl)oxy)methyl)-4-(pyridin-3-ylethynyl)-7-azabicyclo[2.2.1]heptane-7-carboxylate in accordance with the procedure in Example 49.

### Example 49: (R)-(3-Fluorophenyl)(4-(2-(pyridin-3-yl)ethyl)-7-azabicyclo[2.2.1]heptan-1-yl)methanol dihydrochloride

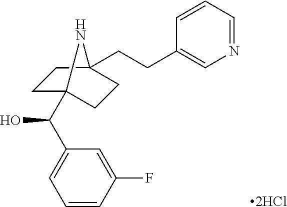 [SMILES: O[C@H](c1cccc(F)c1)C12CCC(CCc3cccnc3)(CC1)N2 ; "·2HCl"]

The title compound is prepared from tert-butyl 1-(((tert-butyldimethylsilyl)oxy)methyl)-4-(pyridin-3-ylethynyl)-7-azabicyclo[2.2.1]heptane-7-carboxylate (prepared from 3-iodopyridine in accordance with the procedure in Example 39, Step (b)) in accordance with the procedure in Example 7, steps (b) to (e), using 3-fluorophenylmagnesium bromide in Step (d), followed by the procedure in Example 37, Step (c).

### Example 50: (S)-(3-Fluorophenyl)(4-(2-(pyridin-3-yl)ethyl)-7-azabicyclo[2.2.1]heptan-1-yl)methanol dihydrochloride

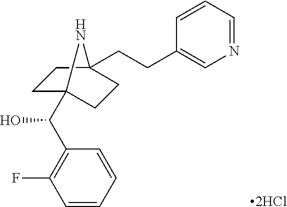 [SMILES: O[C@@H](c1ccccc1F)C12CCC(CCc3cccnc3)(CC1)N2 ; "·2HCl"]

The title compound is prepared from tert-butyl (1r,4r)-1-(((tert-butyldimethylsilyl)-oxy)methyl)-4-(pyridin-3-ylethynyl)-7-azabicyclo[2.2.1]heptane-7-carboxylate in accordance with the procedure in Example 47.

### BIOLOGICAL EXAMPLES

L6-myoblasts were grown in Dulbecco's Modified Eagle's Medium (DMEM) containing 1 g/L glucose supplemented with 10% fetal bovine serum, 2 mM L-Glutamine, 50 U/ml penicillin, 50 μg/mL streptomycin and 10 mM HEPES. Cells were plated at 1×10⁵ cells per mL in 24-well plates. After reaching 90% confluence the cells were grown in medium containing 2% FBS for 7 days where upon cells differentiated into myotubes.

### Biological Example 1: Glucose Uptake

Differentiated L6-myotubes were serum-starved overnight in medium containing 0.5% fatty-acid free BSA and stimulated with an agonist, with a final concentration of 1×10⁻⁵ M. After 1 h 40 min the cells were washed with warm glucose free medium or PBS twice and another portion of agonist was added to the glucose free medium. After 20 min the cells were exposed to 50 nM ³H-2-deoxyglucose for another 10 min before washed in ice cold glucose free medium or PBS three times and lysed in 400 μL/well 0.2 M NaOH for 1 h at 60° C. The cell lysate was mixed with 4 mL scintillation buffer (Emulsifier Safe, Perkin Elmer) and the radioactivity was detected in a β-counter (Tri-Carb 4810TR, Perkin Elmer). The activity for each compound is compared to that of isoproterenol. If a compound at 10 UM shows activity of more than 75% of that of isoproterenol at 10 UM, the activity is denoted with +++; if it is between 75 and 50% it is denoted with ++; if it is between 50 and 25% it is denoted with +; if it less than 25% it is denoted with −.

### Biological Example 2: Measurement of Intracellular CAMP Levels

Differentiated cells were serum-starved overnight and stimulated with an agonist, final concentration 1×10⁻⁵ M, for 15 min in stimulation buffer (HBSS supplemented with 1% BSA, 5 mM HEPES and 1 mM IBMX, pH 7.4). The medium was aspirated and 100 μL of 95% EtOH was added to each well of the 24-well plate and cells were kept at −20° C. overnight. The EtOH was allowed to evaporate and 500 μL of lysis buffer (1% BSA, 5 mM HEPES and 0.3% Tween-20, pH 7.4) was added to each well. The plate was kept at −80° C. for 30 min and then at −20° C. until the day of detection when the samples were thawed. Intracellular cAMP levels were detected using an alpha screen CAMP kit (6760635D from Perkin Elmer). The activity for each compound is compared to that of isoproterenol. If a compound at 10 UM shows activity of more than 75% of that of isoproterenol at 10 UM, the activity is denoted with +++; if it is between 75 and 50% it is denoted with ++; if it is between 50 and 25% it is denoted with +; if it less than 25% it is denoted with −.

Using the assays described in Biological Examples 1 and 2 the following results were obtained.

| Compound | Biological | Biological |
|---|---|---|
| example no. | example 1 | example 2 |
| 1 | +++ | − |
| 2 | ++ | − |
| 3 | +++ | + |
| 4 | + | − |
| 5 | +++ | + |
| 6 | ++ | − |
| 7 | +++ | + |
| 8 | ++ | − |
| 9 | +++ | − |
| 10 | ++ | − |
| 11 | +++ | − |
| 12 | ++ | − |
| 13 | +++ | ++ |
| 14 | ++ | − |
| 15 | − | − |
| 16 | − | − |
| 17 | ++ | − |
| 18 | − | − |
| 19 | − | − |
| 20 | − | − |
| 21 | + | − |
| 22 | + | − |
| 23 | ++ | − |
| 24 | ++ | − |
| 25 | ++ | − |
| 26 | ++ | − |
| 27 | ++ | − |
| 28 | ++ | − |
| 29 | +++ | − |
| 30 | + | − |
| 31 | +++ | − |
| 32 | − | − |
| 33 | ++ | + |
| 34 | ++ | − |
| 35 | +++ | + |
| 36 | + | − |
| 37 | − | − |
| 38 | − | − |
| 39 | ++ | − |
| 40 | − | − |
| 41 | ++ | + |
| 42 | − | − |
| 43 | | |
| 44 | | |
| 45 | | |
| 46 | | |
| 47 | | |
| 48 | | |
| 49 | | |
| 50 | | |

### Biological Example 3: Glucose Uptake in the Presence of the β₂-Antagonist ICI-118,551

Confirmation that glucose uptake is mediated by activation of the β₂-adrenergic receptor may be provided by observation of a reduction (or absence) of glucose uptake in the presence of a β₂-antagonist (ICI-118,551).

Differentiated L6-myotubes are serum-starved overnight in medium containing 0.5% fatty-acid free BSA and incubated with the β₂-adrenergic receptor antagonist ICI-118,551 at a final concentration of 1×10⁻⁵ M for 30 min. The cells are stimulated with a compound of the invention, at a final concentration of 1×10⁻⁵ M. After 1 h 40 min the cells are washed twice with warm, glucose free medium or PBS and additional portions of the compound of the invention and the antagonist are added. After 20 min the cells are exposed to 50 nM 3H-2-deoxyglucose for 10 min before being washed with ice cold glucose free medium or PBS three times and lysed with 0.2 M NaOH, 400 L/well, for 1 h at 60° C. The cell lysate is mixed with 4 mL scintillation buffer (Emulsifier Safe, Perkin Elmer) and the radioactivity is detected in a β-counter (Tri-Carb 4810TR, Perkin Elmer). The activity for each compound is compared to that of isoproterenol. If a compound shows activity of more than 75% of that of isoproterenol at 10 UM, the activity is denoted with +++, if it is between 75 and 50% it is denoted with ++; if it is between 50 and 25% it is denoted with +; if it less than 25% it is denoted with −.

1. A compound of formula I 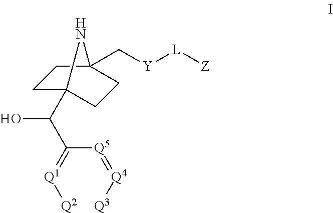
or a pharmaceutically acceptable salt thereof, wherein:
the ring comprising Q¹ to Q⁵ represents
a phenyl optionally substituted with one or more X¹, or
a 5- or 6-membered heteroaryl optionally substituted with one or more X²;
each X¹ and X² independently represents halo, R^{a}, —CN, —N₃, —N(R^{b})R^{c}, —NO₂, —OR^{d}, or —S(O)ₚR^{e};
R^{a} represents C₁₋₆ alkyl, C₂₋₆ alkenyl or C₂₋₆ alkynyl optionally substituted by one or more group selected from halo and ═O;
each R^{b} and R^{d} independently represents H or C₁₋₆ alkyl, C₂₋₆ alkenyl or C₂₋₆ alkynyl optionally substituted by one or more group selected from halo and ═O;
each R^{c} and R^{e} independently represents H or C₁₋₆ alkyl, C₂₋₆ alkenyl or C₂₋₆ alkynyl optionally substituted by one or more halo;
or alternatively R^{b} and R^{c} may be linked together to form, together with the nitrogen atom to which they are attached, a 4- to 6-membered ring, which ring optionally contains one further heteroatom and which ring optionally is substituted by one or more group selected from halo and ═O;
Y represents a direct bond or —O—;
L represents
a direct bond,
or a linear or branched C₁₋₁₂ alkylene, linear or branched C₂₋₁₂ alkenylene or linear or branched C₂₋₁₂ alkynylene;
Z represents
(i) C₃₋₈ cycloalkyl, optionally substituted by one or more groups independently selected from G¹,
(ii) aryl optionally substituted by one or more groups independently selected from G², or
(iii) heteroaryl optionally substituted by one or more groups independently selected from G³;
each G¹ independently represents halo, R^{a1}, —CN, —N(R^{b1})R^{c1}, —OR^{d1}, —S(O)ₚR^{e1}, —S(O)_{q}N(R^{f1})R^{g1}, —N(R^{h1})S(O)ₜRⁱ¹ or ═O;
R^{a1} represents C₁₋₆ alkyl, C₂₋₆ alkenyl or C₂₋₆ alkynyl optionally substituted by one or more group selected from halo and ═O;
each R^{b1} and R^{d1} independently represents H or C₁₋₆ alkyl, C₂₋₆ alkenyl or C₂₋₆ alkynyl optionally substituted by one or more group selected from halo and ═O;
each R^{c1}, R^{e1}, R^{f1}, R^{g1} and R^{h1} independently represents H or C₁₋₆ alkyl, C₂₋₆ alkenyl or C₂₋₆ alkynyl optionally substituted by one or more halo;
Rⁱ¹ represents C₁₋₆ alkyl, C₂₋₆ alkenyl or C₂₋₆ alkynyl optionally substituted by one or more halo;
or alternatively any of R^{b1} and R^{c1} and/or R^{f1} and R^{g1} may be linked together to form, together with the nitrogen atom to which they are attached, a 4- to 6-membered ring, which ring optionally contains one further heteroatom and which ring optionally is substituted by one or more groups independently selected from halo and C₁₋₃ alkyl optionally substituted by one or more group selected from halo and ═O;
each G² and G³ independently represents halo, R^{a2}, —CN, —N₃, —N(R^{b2})R^{c2}, —NO₂, —OR^{d2}, —S(O)ₚR^{e2}, —S(O)_{q}N(R^{f2}R^{g2}) or —N(R^{h2})S(O)ₜRⁱ²;
R^{a2} represents C₁₋₆ alkyl, C₂₋₆ alkenyl or C₂₋₆ alkynyl optionally substituted by one or more group selected from halo and ═O;
each R^{b2} and R^{d2} independently represents H or C₁₋₆ alkyl, C₂₋₆ alkenyl or C₂₋₆ alkynyl optionally substituted by one or more group selected from halo and ═O;
each R^{c2}, R^{e2}, R^{f2}, and R^{g2} independently represents H or C₁₋₆ alkyl, C₂₋₆ alkenyl or C₂₋₆ alkynyl optionally substituted by one or more halo;
R^{h2} independently represents H or C₁₋₆ alkyl, C₂₋₆ alkenyl or C₂₋₆ alkynyl optionally substituted by aryl and/or one or more halo, wherein the aryl is optionally substituted by one or more groups independently selected from G⁴;
Rⁱ² represents C₁₋₆ alkyl, C₂₋₆ alkenyl or C₂₋₆ alkynyl optionally substituted by one or more halo;
or alternatively any of R^{b2} and R^{c2} and/or R^{f2} and R^{g2} may be linked together to form, together with the nitrogen atom to which they are attached, a 4- to 6-membered ring, which ring optionally contains one further heteroatom and which ring optionally is substituted by one or more groups independently selected from halo and C₁₋₃ alkyl optionally substituted by one or more group selected from halo and ═O;
each G⁴ independently represents halo, R^{a3}, —CN, —N₃, —N(R^{b3})R^{c3}, —NO₂, —OR^{d3}, —S(O)ₚR^{e3}, —S(O)_{q}N(R^{f3}R^{g3}) or —N(R^{h3})S(O)+Rⁱ³;
R^{a3} represents C₁₋₆ alkyl, C₂₋₆ alkenyl or C₂₋₆ alkynyl optionally substituted by one or more group selected from halo and ═O;
each R^{b3} and R^{d3} independently represents H or C₁₋₆ alkyl, C₂₋₆ alkenyl or C₂₋₆ alkynyl optionally substituted by one or more groups selected from halo and ═O;
each R^{c3}, R^{e3}, R^{f3}, R^{g3} and R^{h3} independently represents H or C₁₋₆ alkyl, C₂₋₆ alkenyl or C₂₋₆ alkynyl optionally substituted by one or more halo;
Rⁱ³ represents C₁₋₆ alkyl, C₂₋₆ alkenyl or C₂₋₆ alkynyl optionally substituted by one or more halo;
each p independently represents 0, 1 or 2;
each q independently represents 1 or 2;
each t independently represents 1 or 2;
wherein, unless otherwise stated, alkyl, alkenyl and alkynyl groups may be linear or branched, and alkyl and alkenyl groups may also be cyclic or part-cyclic, as appropriate,
and wherein, when Y is —O— and L is a direct bond, Z represents
C₃₋₈ cycloalkyl, optionally substituted by one or more groups independently selected from G¹, or
heteroaryl optionally substituted by one or more groups independently selected from G³.

2. The compound according to any one of the preceding claims, wherein the ring comprising Q¹ to Q⁵ represents
a phenyl optionally substituted with one or more X¹, or
a pyridyl optionally substituted with one or more X².

3. The compound according to any one of the preceding claims, wherein each X¹ and X² independently represents halo, R^{a}, —CN, —N(R^{b})R^{c} or —OR^{d}.

4. The compound according to any one of the preceding claims, wherein:
each R^{a} independently represents C₁₋₆ alkyl optionally substituted by one or more group selected from halo and ═O; and/or
each R^{b}, R^{c} and R^{d} independently represent H or C₁₋₆ alkyl optionally substituted by one or more group selected from halo and ═O.

5. The compound according to any one of the preceding claims, wherein L represents:
a direct bond,
or a linear or branched C₁₋₃ alkylene.

6. The compound according to any one of the preceding claims, wherein Z represents:
(i) cyclohexyl, optionally substituted by one or more groups independently selected from G¹; or
(ii) phenyl optionally substituted by one or more groups independently selected from G².

7. The compound according to any one of the preceding claims, wherein:
each G¹ independently represents —N(R^{b1})R^{c1} or —OR^{d1}; and/or
each G² and/or G³ independently represents halo, R^{a2}, —N(R^{b2})R^{c2}, —OR^{d2} or —N(R^{h2})S(O)ₜRⁱ².

8. A compound as defined in any one of claims 1 to 7 for use in medicine.

9. A pharmaceutical composition comprising a compound as defined in any one of claims 1 to 7, and optionally one or more pharmaceutically acceptable adjuvant, diluent and/or carrier.

10. A compound as defined in any one of claims 1 to 7, for use in the treatment of hyperglycemia or a disorder characterized by hyperglycaemia.

11. The use of a compound as defined in any one of claims 1 to 7, for the manufacture of a medicament for the treatment of hyperglycemia or a disorder characterized by hyperglycaemia.

12. A method of treating hyperglycaemia or a disorder characterized by hyperglycaemia comprising administering to a patient in need thereof a therapeutically effective amount of a compound as defined in any one of claims 1 to 7.

13. The compound for use, method or use according to any one of claims 10 to 12, wherein the hyperglycemia or disorder characterised by hyperglycemia is, or is characterised by, the patient displaying severe insulin resistance.

14. The compound for use, method or use according to any one of claims 10 to 13, wherein the disorder characterised by hyperglycemia is selected from the group consisting of Type 2 diabetes, Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

15. A compound as defined in anyone of claims 1 to 7, for use in the treatment of a non-alcoholic fatty liver disease.

16. The use of a compound as defined in any one of claims 1 to 7, in the manufacture of a medicament for the treatment or prevention of a non-alcoholic fatty liver disease.

17. A method of treating or preventing a non-alcoholic fatty liver disease as defined in comprising administering to a patient in need thereof a therapeutically effective amount of a compound as defined in any one of claims 1 to 7.

18. A compound as defined in anyone of claims 1 to 7, for use in treating a disease or disorder the treatment of which is mediated by activation of the β₂ adrenergic receptor.

19. The use of a compound as defined in any one of claims 1 to 7, in the manufacture of a medicament for use in treating a disease or disorder the treatment of which is mediated by activation of the β₂ adrenergic receptor.

20. A method of treating a disease or disorder the treatment of which is mediated by activation of the β₂ adrenergic receptor comprising administering to a patient in need thereof a therapeutically effective amount of a compound as defined in any one of claims 1 to 7.
